(19) United States
(12) Patent Application Publication
(10) Pub. No.: US 2026/0294321 A1
(43) Pub. Date: Oct. 1, 2026

(54) System and Method for Bilateral Communication Between Humans and Non-Human Animals Using Neural Interfaces

(71) Applicant: QOMPLX LLC, Reston, VA (US)

(72) Inventors: Jason Crabtree, Vienna, VA (US); Richard Kelley, Woodbridge, VA (US); Jason Hopper, Halifax (CA); David Park, Fairfax, VA (US)

(21) Appl. No.: 19/094,808

(22) Filed: Mar. 28, 2025

Related U.S. Application Data
(63) Continuation-in-part of application No. 19/078,192, filed on Mar. 12, 2025.

(51) Int. Cl. A61B 5/372; B82Y 15/00; G06F 3/01; H04W 4/80

(52) U.S. Cl. CPC A61B 5/372; B82Y 15/00; G06F 3/015; H04W 4/80; G06F 2203/011

(57) ABSTRACT

Disclosed embodiments provide a system and method for enabling real-time bilateral neural communication between humans and non-human animals using a combination of non-invasive neural interfaces with optional implantable neural enhancements. Advanced multi-modal pattern recognition algorithms are utilized for real-time neural signal processing, incorporating surface-level sensors and/or enhanced neural monitoring capabilities. These signals are processed through a sophisticated translation unit that converts neural patterns into meaningful communications, while simultaneously providing feedback mechanisms that may deliver precise haptic, auditory, or neural stimulation patterns for reinforcement and response.

### CROSS-REFERENCE TO RELATED APPLICATIONS

Priority is claimed in the application data sheet to the following patents or patent applications, each of which is expressly incorporated herein by reference in its entirety: 19/078,192

### BACKGROUND OF THE INVENTION

### Field of the Art

The present invention relates to the field of bilateral communication systems. More specifically, the invention pertains to neural interface-based systems and methods that enable bilateral communication between humans and non-human animals.

### Discussion of the state of the Art

Humans and animals have shared a symbiotic relationship throughout history, working together in ways that have shaped civilizations and daily life. This partnership often stems from mutual benefit, with humans providing care and shelter while animals assist in labor, transportation, companionship, and survival. Horses revolutionized transportation, enabling people to travel faster and over greater distances. Historically, horses were also central to warfare, pulling chariots, carrying soldiers, and serving as a vital component of cavalry units. Additionally, mules have been used for hauling heavy loads in mining, agriculture, and construction, especially in rugged terrains where machinery is impractical. In a similar manner, oxen have been critical in farming, pulling plows and carts to cultivate large areas of land, especially before the advent of modern machinery. Dogs have been indispensable in hunting, helping track and retrieve game. Dogs also have been used for guarding livestock, property, and also as loyal companions. These partnerships highlight how humans have harnessed the unique abilities of different animals to overcome challenges, build societies, and advance technologies.

### SUMMARY OF THE INVENTION

The inventor has conceived and reduced to practice a system and method for bilateral communication between humans and non-human animals using neural interfaces. The system comprises a computer system with hardware memory configured to execute software instructions that receive non-human animal brainwave data and process it through a machine-learning system. The machine-learning system isolates neural patterns in the brainwave data, associates these patterns with an emotional state of the non-human animal, and computes a confidence score for the emotional state. When the confidence score exceeds a predetermined threshold, the system renders and presents an audio/visual form of the emotional state on an output device, enabling humans to understand the non-human animal’s emotional state.

In an embodiment, the system further comprises a non-invasive brainwave sensor configured to obtain brainwave data from a non-human animal and provide this data to the computer system.

In an embodiment, the non-invasive brainwave sensor comprises a sensor made of conductive polymer ink.

In an embodiment, the conductive polymer ink comprises (Poly(3,4-ethylenedioxythiophene): poly(styrenesulfonate)) (PEDOT:PSS), providing enhanced conductivity and biocompatibility.

In an embodiment, the non-invasive brainwave sensor comprises an epidermal tattoo sensor that adheres directly to the animal’s skin.

In an embodiment, the epidermal tattoo sensor comprises carbon nanotubes (CNTs), enabling improved signal detection capabilities.

In an embodiment, the epidermal tattoo sensor comprises gold nanomaterials for enhanced conductivity and signal quality.

In an embodiment, the non-invasive brainwave sensor further comprises a wireless data transmission module for transmitting the acquired brainwave data without physical connection constraints.

In an embodiment, the wireless data transmission module includes a Bluetooth Low Energy (BLE) module, providing energy-efficient wireless communication.

In an embodiment, the machine-learning system includes a large language model (LLM) for advanced pattern recognition and interpretation of neural signals.

In an embodiment, the LLM includes a multi-head attention (MHA) mechanism that enables the model to focus on different aspects of the neural data simultaneously, improving accuracy in emotional state detection.

In an embodiment, the computer system is further configured to receive and process brainwave data from multiple non-human animals, coordinate task assignments among them based on their detected emotional states, and transmit adaptive commands to redistribute tasks in response to changes in cognitive load, stress levels, or environmental conditions.

In an embodiment, the machine-learning system is further configured to receive auxiliary sensor data comprising at least one of physiological data, motion data, audio data, video data, or thermal imaging data from the non-human animal; process this auxiliary data in conjunction with the brainwave data using cross-modal fusion techniques; and adjust confidence scores for emotional state determinations based on correlations between the brainwave data and the auxiliary sensor data.

In an embodiment, computing the confidence score further comprises dynamically adjusting a confidence threshold based on factors such as environmental context, time of day, the non-human animal’s baseline patterns, recent activity history, or signal quality metrics; applying this dynamically adjusted confidence threshold to filter emotional state determinations; and progressively refining the confidence threshold through continuous learning from feedback data comprising successful and unsuccessful emotional state interpretations.

### BRIEF DESCRIPTION OF THE DRAWING FIGURES

FIG. 1 shows an exemplary environment in which a system for multimodal orchestration for human-animal-robot collaborative task execution may be used, in accordance with one or more embodiments.

FIG. 2 is a block diagram illustrating components of a system for multimodal orchestration for human-animal-robot collaborative task execution, in accordance with one or more embodiments.

FIG. 3 is a block diagram illustrating details of a neural interface component, in accordance with one or more embodiments.

FIG. 4 is a block diagram illustrating details of a translation processing unit, in accordance with one or more embodiments.

FIG. 5 is a block diagram illustrating details of a multi-species output unit, in accordance with one or more embodiments.

FIG. 6 is a block diagram illustrating details of a multi-species collaboration layer, in accordance with one or more embodiments.

FIG. 7 is a block diagram illustrating details of a large language model (LLM) or other AI, symbolic reasoning, or neurosymbolic orchestration system, in accordance with one or more embodiments.

FIG. 8 is a block diagram illustrating details of a Simultaneous Localization and Mapping (SLAM) system, in accordance with one or more embodiments.

FIG. 9 is a block diagram illustrating an exemplary training system for multimodal orchestration for human-animal-robot collaborative task execution, in accordance with one or more embodiments.

FIG. 10 shows an exemplary environment in which a system for bilateral communication between humans and non-human animals may be used, in accordance with one or more embodiments.

FIG. 11 shows a block diagram of an exemplary non-invasive sensor, in accordance with one or more embodiments.

FIG. 12 is a block diagram illustrating components of a system for bilateral communication between humans and canines, in accordance with one or more embodiments.

FIG. 13 shows exemplary canine brainwaves that may be analyzed using one or more embodiments.

FIG. 14 is a flow diagram illustrating an exemplary method for bilateral communication between humans and non-human animals, in accordance with one or more embodiments.

FIG. 15 is a flow diagram illustrating an exemplary method for training a system for bilateral communication between humans and non-human animals, according to one or more embodiments.

FIG. 16 is a method diagram illustrating a comprehensive process flow from data acquisition to real-time inference for bilateral human-animal communication.

FIG. 17 illustrates an exemplary computing environment on which an embodiment described herein may be implemented, in full or in part.

FIG. 18 is a block diagram illustrating a transfer learning for neural-based interspecies communication system, in accordance with one or more embodiments.

FIG. 19 is a block diagram illustrating a bilateral neural-based interspecies communication system with transfer learning.

FIG. 20 is a block diagram illustrating a robust multi-modal fusion engine for an interspecies communication system.

FIG. 21 is a block diagram illustrating an advanced IoT integration framework for neural-based animal communication.

The drawings are not necessarily to scale. The drawings are merely schematic representations, not intended to portray specific parameters of the disclosed embodiments. The drawings are intended to depict only typical embodiments of the invention, and therefore should not be considered as limiting in scope.

### DETAILED DESCRIPTION OF THE INVENTION

The inventor has conceived and reduced to practice a system and method enabling bilateral communication between humans and non-human animals through neural interfaces. This approach facilitates understanding and interaction across species barriers through advanced technology and signal processing.

The system comprises a comprehensive system utilizing non-invasive neural interfaces to detect, process, and interpret brainwave signals from non-human animals. These neural interfaces may be implemented through various means, including but not limited to, conductive polymer-based sensors, epidermal tattoo-like applications, or other suitable brainwave detection technologies that may effectively capture neural activity without causing discomfort to the animal.

When applied to an animal subject, these non-invasive sensors capture brainwave data, which is then transmitted to a processing system. Transmission may occur through wireless communication protocols, enabling freedom of movement for the animal while maintaining continuous data collection. The processing system employs sophisticated machine learning algorithms to analyze the received neural signals.

The machine learning component of the system performs multiple functions on the acquired brainwave data. Initially, it isolates specific neural patterns within the complex brainwave information. These patterns are then associated with particular emotional states through trained models that have learned correlations between neural signatures and emotional or cognitive conditions. The system calculates confidence scores for these associations, providing a measure of certainty regarding the interpreted emotional state.

When the system determines that a particular emotional state has been identified with sufficient confidence—exceeding a predetermined threshold—it renders this information in a format comprehensible to human users. This may include visual displays, auditory feedback, or other suitable output modalities that effectively communicate the animal’s emotional state to human observers or handlers.

The invention’s machine learning capabilities may incorporate advanced modeling techniques, including large language models with multi-head attention mechanisms. These sophisticated algorithms enable the system to process complex neural data patterns and improve interpretation accuracy by focusing simultaneously on multiple aspects of the input data.

Beyond individual animal monitoring, the invention extends to multi-animal scenarios, where brainwave data from multiple non-human animals may be simultaneously processed. This capability enables coordination of task assignments based on detected emotional states and adaptive redistribution of tasks in response to changing conditions, including variations in cognitive load, stress levels, or environmental factors.

The system’s sensing capabilities may be enhanced through auxiliary data collection, incorporating physiological metrics, motion information, audio recordings, video footage, or thermal imaging. This multi-modal approach allows for cross-referencing between brainwave patterns and other observable behaviors or conditions, improving the accuracy of emotional state determinations.

Furthermore, the system may implement dynamic confidence thresholds that adapt based on contextual factors including environmental conditions, time patterns, baseline neural signatures specific to individual animals, recent activity patterns, or signal quality metrics. This adaptive approach ensures optimal performance across varying conditions and subjects, with the system continuously learning and refining its interpretative capabilities through ongoing feedback mechanisms.

The bilateral nature of the invention also encompasses methods for conveying information from humans to animals. Human inputs—whether through direct interface, verbal commands, or other means—may be translated into signals or stimuli that animals may comprehend, including auditory, haptic, or other suitable feedback mechanisms.

The system provides a transformative communication framework applicable across various domains, including but not limited to animal training, service animal applications, wildlife research, veterinary care, and human-animal collaborative tasks. By bridging the communication gap between humans and non-human animals, the invention enables enhanced understanding, cooperation, and interaction between species.

### Detailed Description of Wearable Sensors

The non-invasive brainwave sensors described herein may be implemented through various embodiments, including conductive polymer-based sensors and epidermal tattoo-like applications. These sensors represent one component within what may be a more comprehensive integrated sensory system.

In certain implementations, the brainwave sensors may operate as part of a broader multimodal sensory processing framework. While the present invention focuses primarily on neural signal acquisition and interpretation, it is contemplated that future enhancements may incorporate additional sensory modalities to create a more robust cross-species state estimation system.

Such enhancements may include integration with scent-based data acquisition systems capable of detecting and analyzing olfactory signals that complement neural data, potentially providing context for certain brainwave patterns exhibited during scent detection activities. The combination of neural signals with olfactory data streams may provide more comprehensive insights into animal cognitive states, particularly for species with highly developed olfactory capabilities.

Additionally, the wearable sensor system may be designed with consideration for integration with biomechanical and environmental modeling components. Such integration would allow correlation between detected neural patterns and the animal’s physical movements, posture, gait characteristics, and interactions with environmental elements, thereby providing contextual enrichment to neural data interpretation.

Furthermore, the wearable sensors described herein may be positioned to support advanced object recognition capabilities, wherein neural signatures associated with an animal’s perception of objects could be mapped and interpreted within an open-vocabulary framework that allows for flexible categorization and identification across species boundaries.

In more advanced implementations, the wearable sensor system may incorporate components that facilitate simultaneous localization and mapping (SLAM) functionality, allowing for spatial awareness and environmental mapping based on the animal’s movement and perception. Such functionality may utilize point cloud processing techniques to create detailed environmental representations that contextualize the animal’s neural responses to spatial stimuli.

The tattoo and/or wearable sensors in the present disclosure thus represent foundational components that may be expanded into a comprehensive multimodal sensory suite capable of rich cross-species state estimation through the integration of neural data with other sensory and contextual information streams.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to use in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

### Definitions

As used herein, “Canine” refers to members of the Canidae family, which includes domestic dogs (Canis lupus familiaris), and particularly to domestic dogs.

As used herein, “brainwave sensor” refers to a device that detects and records electrical activity in the brain, such as using electroencephalography (EEG) or similar technology. These sensors work by measuring the small voltage fluctuations generated by neural activity and translating them into readable signals.

As used herein, “large language model” (LLM) refers to a type of artificial intelligence model, typically based on deep learning, that is designed to process, understand, and generate human language. These models are trained on massive datasets, enabling them to predict the likelihood of sequences of tokens, understand context, and produce coherent and contextually relevant responses.

As used herein, “attention mechanism” refers to a machine learning technique that enables models to dynamically focus on the most relevant parts of input data when making predictions. Attention mechanisms may be configured to assign different importance (weights) to various parts of the input, helping the model prioritize relevant information. Additionally, attention mechanisms may help models handle long-range dependencies by selectively attending to important information rather than processing everything equally.

### Conceptual Architecture

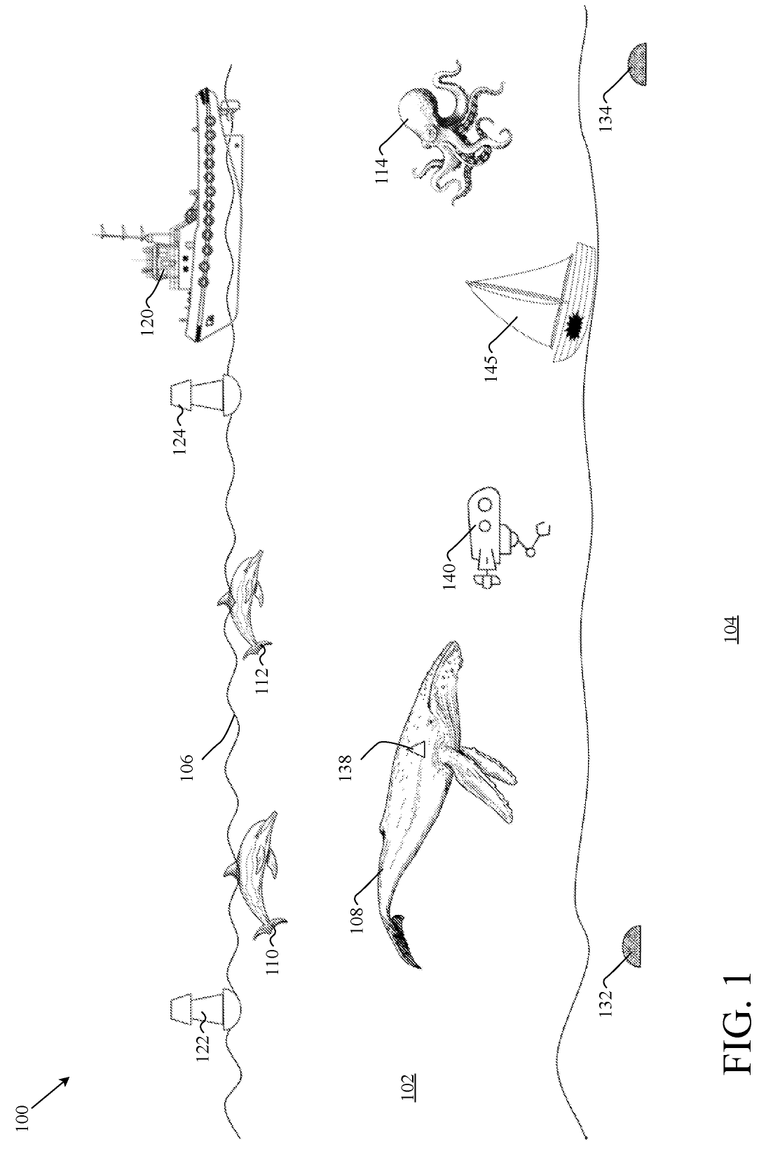

FIG. 1 shows an exemplary environment in which a system for multi-modal orchestration for human-animal-robot collaborative task execution may be used, in accordance with one or more embodiments. Environment 100 may include a large body of water 102. However, disclosed embodiments are not limited to use in aquatic environments. Some embodiments may interact with land animals and/or flying animals and may be used in environments involving aquatic, land-based, and/or above-ground environments. Large body of water 102 may include an ocean (e.g., Pacific Ocean, Atlantic Ocean, etc.), a sea, (e.g., Mediterranean Sea, Baltic Sea, etc.), a lake (e.g., Lake Superior, Lake Victoria, etc.), a river (e.g., Mississippi River), a gulf (e.g., Gulf of Mexico), a bay (e.g., Hudson Bay), a strait (e.g., Bering Strait), a fjord, an estuary, a man-made reservoir, and/or other suitable large body of water.

The environment 100 may include one or more buoys, indicated as 122 and 124 in environment 100. In one or more embodiments, the buoys (122, 124) float on the surface 106 of the body of water 102, and may include a variety of equipment for sensing, receiving, storing, and/or transmitting data, as well as one or more output devices. The buoys may include one or more atmospheric sensors. The atmospheric sensors may include a wind speed sensor, wind direction sensor, air temperature sensor, air humidity sensor, barometric pressure sensor, solar radiation sensor, microphone, and/or other suitable atmospheric sensors.

The buoys may include one or more water-based sensors. The water-based sensors may include temperature sensors (to measure surface water temperature), salinity sensors (to determine the salt content of the water), pH sensors (to measure acidity/alkalinity), dissolved oxygen sensors (to monitor oxygen levels for aquatic life), and/or turbidity sensors (to measure water clarity). The water-based sensors may include wave height and direction sensors to measure ocean swell and surface conditions, current velocity sensors (to track underwater currents), tide and sea level sensors, and/or chlorophyll sensors (to estimate plankton levels and water productivity). The water-based sensors may include underwater microphones to detect underwater sounds from aquatic life and/or marine craft. The buoys may include one or more meteorological sensors, such as rain gauges and/or lightning detectors.

The buoys may include a variety of communication equipment, such as satellite transmitters (e.g., Iridium) for long-range communication. The buoys may include cellular modems, suitable for communication within areas of network coverage. The buoys may include radio transmitters to enable short-range transmission for local stations or vessels. The buoys may include Wi-Fi and/or Bluetooth modules to support local data access when in close proximity. The buoys may include GPS receivers to track buoy position and movement. Other communication systems may be present on the buoys in one or more embodiments. The buoys may include a wide variety of output devices, including, but not limited to, signal lights, audible alarms, underwater speakers, out-of-water speakers, and/or digital displays. The buoys may include a variety of computing devices, such as embedded microcontrollers, edge processors, data loggers, and/or other suitable computing equipment. In one or more embodiments, the buoys may include AI-based edge devices for advanced tasks such as detecting patterns, identifying marine life, and/or predictive analysis.

The environment 100 may further include one or more seafloor detection devices, that are located on seafloor 104, indicated at 132 and 134. Each seafloor detection device (132, 134) may include a seismometer, hydrophone array, and a wide range of other sensors and technologies for monitoring vibrations, seismic activity, and underwater sounds. The seafloor detection device may include a broadband seismometer for capturing a wide range of seismic frequencies. The seafloor detection device may further include a short-period seismometer to focus on high-frequency vibrations. The seafloor detection device may further include one or more accelerometers for measuring ground accelerations for vibrations caused by earthquakes, underwater landslides, or human-made activities such as drilling.

The hydrophone array within the seafloor detection devices may enable detecting soundwaves from marine mammals such as whales and dolphins. Other sounds may also be detected by the hydrophone array. These sounds may include sounds from underwater explosions, ship noise, or submarine movements. One or more hydrophones within the hydrophone array may be tuned for capturing low-frequency sounds from large marine animals and/or geological events. The seafloor detection devices may further include a current meter to track underwater currents that may result from tectonic and/or seismic activity.

In one or more embodiments, the seafloor detection devices may be communicatively coupled to one or more surface devices, such as buoys (122, 124), and/or ship 120. The communicative coupling may include cables, such as copper cables, fiber-optic cables, or the like, between a seafloor detection device and a buoy. The communicative coupling may include wireless communication such as RF-based communication and/or acoustic modems that may transmit data via sound waves to nearby surface buoys, ships, or other underwater devices.

The environment 100 may include ship 120. Ship 120 may contain one or more computing devices, such as a data server, virtualized computing environment, and/or other computing devices for enabling and/or supporting the multi-modal orchestration system of disclosed embodiments. Additionally, ship 120 may provide long range communication to one or more remote servers, via the Internet. In one or more embodiments, ship 120 may be equipped with satellite communication (SATCOM). In embodiments, the ship 120 may be equipped with a satellite antenna. The satellite antenna may enable a connection with a geostationary satellite and/or a low-Earth-orbit (LEO) satellite, that in turn relays data to a ground station connected to the internet. The ship 120 may further be equipped with a cellular transceiver to utilize a cellular network when within the range of coastal areas.

Within the body of water 102, a wide variety of aquatic life may be present. The aquatic life may include one or more dolphins/porpoises, indicated at 110 and 112. These may include the Bottlenose Dolphin (Tursiops truncatus), Common Dolphin (Delphinus delphis), Dusky Dolphin (Lagenorhynchus obscurus), Harbor Porpoise (Phocoena phocoena), Spectacled Porpoise (Phocoena dioptrica), Orca or Killer Whale (Orcinus orca), and/or other varieties of dolphin/porpoise. These intelligent and diverse marine mammals contribute significantly to marine ecosystems and hold a special place in human culture and scientific research. Each species has its unique adaptations to its habitat, from the open ocean to coastal regions and even rivers. In embodiments, communication between the dolphins/porpoises for the multi-modal orchestration system of disclosed embodiments may be accomplished by sending acoustic signals to the dolphins/porpoises and/or receiving acoustic signals from the dolphins/porpoises via buoys (122, 124) and/or seafloor detection devices (132, 134).

The aquatic life within body of water 102 may include one or more octopus/squid, indicated at 114. The octopus may include a Common Octopus (Octopus vulgaris), Giant Pacific Octopus (Enteroctopus dofleini), Mimic Octopus (Thaumoctopus mimicus), and/or other types of octopus. The squids may include a Giant Squid (Architeuthis dux), Colossal Squid (Mesonychoteuthis Hamiltoni), Common Squid (Loligo vulgaris), and/or other types of squids. Both octopuses and squids are among the most intelligent invertebrates, exhibiting behaviors that suggest advanced cognitive abilities, such as learning, problem-solving, and communication. In particular, octopuses have been known for their ability to solve complex puzzles, such as opening jars, navigating mazes, and manipulating objects in creative ways. Octopuses like the common octopus (Octopus vulgaris) have demonstrated learning through observation and trial-and-error. Additionally, some squids may learn to avoid predators by associating specific cues (like the presence of certain predators) with danger. These traits and abilities may be used for enabling multi-modal orchestration for human-animal-robot collaborative task execution.

The aquatic life within body of water 102 may include one or more whales, indicated generally at 108. The whales may include Baleen whales (Mysticeti) and/or toothed whales (Odontoceti). The Baleen whales may include the Blue Whale (Balaenoptera musculus), Humpback Whale (Megaptera novaeangliae), Fin Whale (Balaenoptera physalus), Minke Whale (Balaenoptera acutorostrata), and/or other varieties of Baleen whale. The toothed whales may include a Sperm Whale (Physeter macrocephalus), Beluga Whale (Delphinapterus leucas), Narwhal (Monodon monoceros), and/or other types of toothed whale.

The environment 100 may include one or more marine life wearable electronic devices, such as indicated at 138, affixed to whale 108. The marine life wearable electronic device 138 may include one or more sensors, such as a GPS receiver. The GPS receiver may obtain geolocation data for the whale 108 at times when the whale surfaces. The marine life wearable electronic device 138 may further include one or more acoustic positioning sensors for using triangulation with other devices, such as buoys (122, 124) and/or seafloor detection devices (132, 134) to determine a relative position. The marine life wearable electronic device 138 may further include an accelerometer and gyroscope for tracking movement data, including swimming behavior, diving depth, and/or orientation.

Since whales may communicate via sound, and some whales use echolocation to navigate and find prey, the marine life wearable device 138 may include sensors to detect these sounds or transmit sounds to communicate with the whale 108. The sensors may include a hydrophone to detect vocalizations from the whale (such as songs, calls, or clicks) and record ambient underwater sounds. The marine life wearable device 138 may further include a transducer configured and disposed to emit sounds or vocalizations that may be heard by the whale, facilitating communication and/or behavioral studies. In embodiments, the transducer is tuned to output sounds in frequencies that whales may hear and interact with. In one or more embodiments, the marine life wearable device 138 may further include a haptic module. The haptic module may enable the marine life wearable device 138 to provide tactile feedback to the whale. The haptic vibrations may be delivered through components such as a waterproof vibration device that creates a physical sensation, in order to provide feedback to the whale.

The acoustic communication with whale 108 may include complex vocalizations and communication methods. These sounds may serve various purposes, including navigation, identification, mating, and social interactions. The types of sounds whales produce and the patterns they follow depend on the species, as different whales communicate in different ways. The sounds may include ‘songs.’ These songs are complex, long sequences of sounds that often repeat in patterns and may last for several minutes to hours. Male humpback whales are especially known for their songs, which may be used for mating purposes. The songs consist of different “themes” that are repeated in a specific order and may change over time. The songs may carry for miles underwater, allowing males to attract females or compete with other males.

The sounds may include clicks. The clicks may be short, sharp sounds that are used primarily for echolocation (a form of biological sonar). By emitting clicks and analyzing the returning echoes, whales may navigate and detect prey. In some species, clicks are used for communication, especially in social species like orcas and dolphins, where they may serve to coordinate group behavior or signal social intentions. The sounds may include low-frequency sounds. The low-frequency sounds vary from low-frequency moans and grunts to more intense roars, which are thought to be used for communication over long distances. The sounds are often very deep and may travel hundreds of miles across the ocean. The sounds may include non-vocal sounds, such as tail slaps. Some whales, such as humpback whales, may use physical slaps of their tails (flukes) or pectoral fins to produce sounds that may be used for communication, signaling aggression, or coordinating with others in their group. In one or more embodiments, the sound patterns, and corresponding animal behaviors may be stored in a database or other suitable format to serve as training data for one or more machine learning systems to facilitate interspecies communication between humans and/or one or more non-human animal species.

The marine life wearable device 138 further includes a power source, such as a rechargeable or replaceable battery. In some embodiments, the marine life wearable device 138 may be a disposable device with a one-time use sealed battery, such as a lithium-ion battery. In one or more embodiments, the marine life wearable device 138 may be affixed via a strap to the tail fin, or other appendage of the whale. In embodiments, the strap may be comprised of a biodegradable material that dissolves or decomposes over time, enabling the marine life wearable device 138 to fall off the whale after a period of time, such that the device does not cause any permanent discomfort for the whale. In some embodiments, the marine life wearable device 138 may be affixed via a biodegradable adhesive. The biodegradable adhesive may include a starch-based adhesive and may be formulated to wear off after a period of time, enabling the marine life wearable device 138 to fall off the whale, such that the device does not cause any permanent discomfort for the whale.

The environment 100 may further include an autonomous underwater vehicle 140. The autonomous underwater vehicle 140 may be an electromechanical device that includes an onboard computer for receiving commands and/or data from ship 120, buoys (122, 124), marine life wearable device 138, and/or seafloor detection devices (132, 134). Embodiments may include receiving additional human communication data, and outputting the additional human communication data to one or more electromechanical devices, such as autonomous underwater vehicle 140. Embodiments may include receiving additional human communication data, and outputting the additional human communication data to one or more electronic devices, such as a remote computing device located on ship 120.

The types of tasks performed by the multi-modal orchestration for human-animal-robot collaborative task execution may include search and rescue, exploration, surveillance, and/or other suitable tasks. Environment 100 may include a shipwreck 145. As an example, to determine the precise location of the shipwreck, size of the debris field of the shipwreck, and/or other information, a collaborative task involving humans (e.g., on ship 120), robots (e.g., autonomous underwater vehicle 140), and non-human animals (e.g., whale 108, octopus 114, and/or dolphins 110, 112) may be executed by using AI-enabled interspecies communication techniques, along with Simultaneous Localization and Mapping (SLAM) techniques enabled by sensors, satellite receivers, radar, lidar, RF-based triangulation, and/or other suitable techniques, as will be further described in the description for the figures that follow.

In one exemplary embodiment, the SLAM subsystem is radically enhanced through the integration of a hybrid, multi-modal sensor fusion architecture that unifies deep-learning-based feature extraction with advanced geometric optimization and uncertainty-aware data association techniques. Building upon the core ideas of AirSLAM and SP-SLAM, the system employs a unified point-line network (PLNet) that concurrently detects both keypoints and structural line features under varying illumination conditions, thereby ensuring robust performance even in the presence of dramatic lighting changes. This deep network is augmented with a tri-plane encoding strategy that efficiently captures scene appearance data while preserving geometric fidelity, enabling dense 3D mapping with minimal memory overhead. The extracted features are then fused with inertial and other sensor inputs via lightweight matching algorithms—such as those inspired by LightGlue—to enable real-time visual-inertial odometry, which continuously refines camera pose estimates without relying on traditional keyframe selection.

Simultaneously, the system leverages a plane-based optimization framework reminiscent of the Eigen-Factors approach, where raw 3D point cloud data from LiDAR or RGB-D sensors is aggregated into a compact summation matrix that captures point-to-plane residuals at linear complexity. By decoupling plane estimation from trajectory optimization through a bilevel formulation, the SLAM subsystem achieves rapid convergence and enhanced accuracy even in complex, cluttered environments. Moreover, the incorporation of Bayesian inference techniques—integrating Random Finite Set (RFS) theory—allows the system to model feature uncertainty probabilistically, thereby eliminating the need for heuristic-based data association. This unified approach ensures that ambiguous or occluded features are handled in a statistically robust manner, significantly reducing localization drift and map inconsistency.

Further advancing these capabilities, the SLAM subsystem integrates uncertainty-aware sensor fusion mechanisms that explicitly model the noise characteristics of diverse sensor modalities. For instance, radar measurements are processed using a polar-coordinate uncertainty model that transforms measurement covariances into Cartesian coordinates, while visual sensors benefit from adaptive weighting schemes based on real-time confidence estimates derived from deep-learning predictions. These uncertainty-aware residuals are incorporated into a weighted least-squares optimization framework that dynamically adjusts the influence of each sensor input according to its reliability, ensuring robust performance even in adverse conditions such as low-light, high-dynamic-range, or noisy sensor environments.

In addition, the SLAM framework is further empowered by a multi-modal integration layer that synchronizes data streams from heterogeneous sources—including visible and infrared cameras, acoustic sensors, underwater LiDAR, and electromagnetic sensors—into a coherent spatial-temporal model. Advanced cross-attention mechanisms are employed to correlate features across modalities, yielding a unified representation that is then used to construct and continuously update a dense, real-time map of the environment. This layer also supports a predictive relocalization strategy, wherein a scene-dependent junction vocabulary and directed acyclic graph (DAG) representation of reasoning steps enable rapid recovery from localization failures. As new data is incorporated, the system dynamically refines both the map and the corresponding agent poses, ensuring seamless adaptation to changes in the operational environment.

Collectively, these enhancements—encompassing adaptive deep feature extraction, plane-based bilevel optimization, uncertainty-aware fusion, and multi-modal data integration—yield a SLAM subsystem that not only overcomes the limitations of conventional approaches but also surpasses state-of-the-art systems in terms of accuracy, robustness, and computational efficiency. By integrating these advanced techniques into the multispecies orchestration framework, the invention achieves unprecedented situational awareness and real-time mapping performance, thereby enabling robust, scalable, and resilient coordinated task execution across diverse domains such as terrestrial, maritime, aerial, and space environments.

In one exemplary embodiment, the SLAM subsystem is radically reengineered to integrate a hybrid, multi-modal data fusion pipeline that not only overcomes the limitations of current visual SLAM systems in dynamic environments but also exceeds the capabilities of DVDS and advanced LiDAR–visual–inertial semantic mapping approaches. In this embodiment, the system first deploys a dual-phase dynamic object exclusion mechanism that simultaneously processes visual and LiDAR inputs using a multi-task deep neural network framework. This framework leverages state-of-the-art image classification, object detection, and semantic segmentation algorithms to filter out transient, moving objects from static scene elements prior to feature extraction. By doing so, the system isolates reliable features even in environments with heavy occlusions, low-texture regions, or rapidly changing illumination, thereby preventing dynamic interference from corrupting downstream optical flow estimation and point cloud registration.

Once dynamic objects are removed, the filtered data is fed into an enhanced transformer-based feature aggregation module—termed the Dispersive Transformer (DisFormer)—which builds on the concept of Top-K Sparse Attention (TKSA) and Mixed-Scale Feed-Forward Networks (MSFN). DisFormer is designed to extract robust, high-dimensional feature representations from both dense visual frames and sparse LiDAR scans by selectively focusing on the most informative signal components while discarding redundant information. This novel transformer module is seamlessly integrated with a gated recurrent unit (GRU) that iteratively refines pose estimates through dense bundle adjustment, effectively combining temporal information with deep semantic cues to continuously update camera and sensor trajectories in real time.

Further distinguishing this embodiment, an object-level semantic mapping layer is incorporated to handle complex, natural environments such as forests, urban scenes, and industrial settings where GNSS signals are unreliable. This layer employs innovative cluster-block data structures that perform object-level segmentation and tracking; for example, in forested environments, individual tree trunks are segmented from LiDAR point clouds and associated with semantic labels obtained from corresponding visual data. These object-level features are then incorporated into a global optimization framework that minimizes mapping drift by enforcing consistency constraints across multiple frames and sensor modalities. This robust semantic mapping capability not only enhances localization accuracy but also provides rich contextual information that may be used for subsequent interspecies coordination and task execution.

To ensure the system operates in real time on embedded platforms, advanced uncertainty-aware sensor fusion techniques are deployed. Each sensor input—whether visual, LiDAR, inertial, or radar—is assigned a dynamically computed confidence score based on its noise characteristics and environmental conditions. These confidence metrics modulate the weighting of individual sensor contributions within a weighted least-squares optimization framework, thereby enhancing robustness to sensor noise, illumination changes, and partial occlusions. By leveraging GPU-based acceleration and efficient inference engines, the entire SLAM pipeline is optimized for low latency, enabling continuous, real-time mapping and localization even under challenging dynamic conditions.

Collectively, these innovations yield a SLAM subsystem that not only filters dynamic elements and robustly extracts discriminative features using novel transformer-based methods but also integrates object-level semantic understanding and uncertainty-aware sensor fusion into a unified, adaptive mapping framework. This comprehensive approach significantly reduces pose estimation errors and mapping drift, while also providing the high-fidelity, context-rich spatial data necessary for coordinated task execution across heterogeneous domains—including terrestrial, maritime, aerial, and even space environments—thereby setting a new benchmark for real-world SLAM performance.

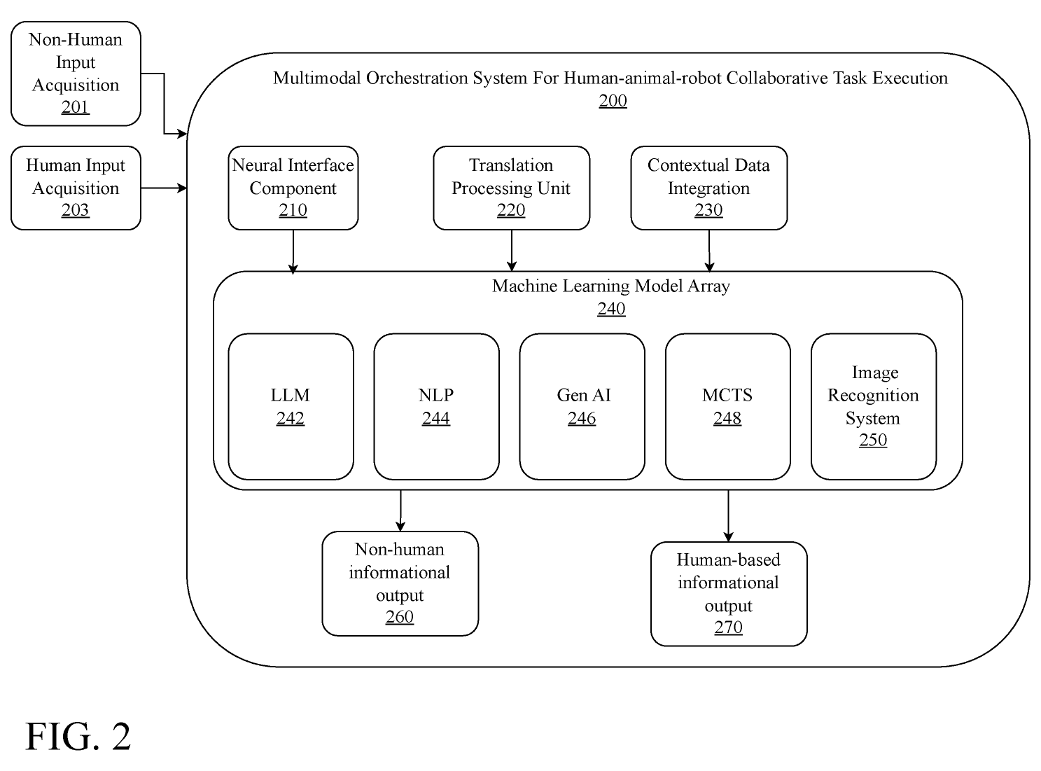

FIG. 2 is a block diagram illustrating components of a system for multimodal orchestration for human-animal-robot collaborative task execution, in accordance with one or more embodiments. System 200 may receive as input, non-human input acquisition 201, and human input acquisition 203. The non-human input acquisition 201 may include input from animals. The input may include audio input. The audio input may include vocalizations such as songs, clicks, chirps, groans, roars, and the like. The audio input may include phonemes and/or words, such as from certain species of birds that are capable of mimicking and producing phonemes from human languages. The audio input may include non-vocal sounds such as tapping or banging sounds from tapping limbs, appendages, or the like. The non-human input acquisition 201 may further include visual information such as sign language gestures, such as may be performed by various primates. The human input acquisition 203 may include spoken language, text input, sign language, and/or other suitable input. The non-human input acquisition 201 and human input acquisition 203 are input to the system 200 for multimodal orchestration for human-animal-robot collaborative task execution, and the resulting output may include a non-human informational output 260, and a human-based informational output 270, thereby facilitating interspecies communication.

The system 200 may include a neural interface component 210. The neural interface component 210 may enable the detection of nuanced neural responses from animals that indicate social, emotional, and environmental interactions. The animals may include land animals, such as horses, cats, and dogs. The animals may include aquatic animals, such as whales, dolphins, fish, octopus, and squid. The animals may include birds and other flying animals. In embodiments, the neural interface component 210 may be coupled to the animals to obtain signals indicative of emotional states, and/or other communication patterns. The system 200 may include a translation processing unit 220. The translation processing unit 220 may utilize machine learning models which are trained to correlate neural patterns of animals to known behaviors, vocalizations, and intentions. The system 200 may include a contextual data integration module 230. The contextual integration module 230 may combine modalities (such as neural signals, vocalizations, gestural data, and/or scent vectors) in a multimodal fusion layer. A sliding time window provides temporal alignment, associating changes in scent concentration with concurrent neural or behavioral shifts. The outputs of the neural interface component 210, translation processing unit 220, and/or contextual data integration model 230 are input to machine learning model array 240.

Machine learning model array 240 may include one or more machine learning models, neural networks, and/or other systems for processing and interpreting input data. The machine learning model array 240 may include a large language model 242. The large language model (LLM) 242 may be trained for specific animals (e.g., species-specific or even individual-specific) and may ingest continuous streams of neural population data recorded across multiple tasks and states. These models go beyond simple language: they become multimodal encoders of animal neural signals, motor outputs, observed behaviors, and contextual cues. By structuring training data to include “high-incentive” versus “neutral” tasks, the LLM may learn when the animal’s neural signature deviates from its optimal preparatory patterns. In embodiments, the machine-learning system includes a large language model (LLM).

The machine learning model array 240 may include a natural language processing (NLP) module 244. The NLP module 244 may enable the conversion of human speech to animal-understandable patterns. The NLP module 244 may include NLP pipelines that parse human language into semantic tokens. These tokens may then be mapped onto a species-specific “neural command embedding space.” For whales, this might involve converting a request such as ‘swim to the surface’ into a neural stimulation pattern, along with an auditory output pattern such as a song or pattern of clicks. For canines, this might involve converting a request like “Fetch the red ball” into a neural stimulation pattern plus a subtle auditory or tactile cue that aligns with the dog’s pre-trained internal representations of the action “fetch” and the visual concept “red ball.”

The machine learning model array 240 may include a generative artificial intelligence (Gen AI) module 246. The Gen AI module 246 may enable supplementing training data with synthesized data, such as vocal data (e.g., canine vocalizations or whale codas), where the vocal data is created with properties such as number and regularity of signal units (clicks, barks), spectral means, and/or amplitude envelopes. The Gen AI module 246 may include a generative adversarial network (GAN), such as WaveGAN, InfoGAN, fiwGAN, and/or other suitable GAN.

The machine learning model array 240 may include a Monte Carlo Tree Search (MCTS) module 248. The MCTS module 248 may enable adaptive, look-ahead scheduling decisions. Instead of applying fixed heuristics or static load-balancing, disclosed embodiments may simulate and/or evaluate multiple future states of the pipeline before choosing the next action. By repeatedly exploring and exploiting different pipeline routing decisions (e.g., which specialist model to send partial outputs to, or how to scale certain pipeline segments), MCTS may minimize the cumulative regret over time, converging toward near-optimal scheduling policies that are robust to changing conditions, input distributions, and latency constraints. In one or more embodiments, the MCTS module 248 may enable enhanced resource allocation, such as allocating more GPUs, selecting specialized hardware accelerators, and/or adjusting batch sizes downstream.

The machine learning model array 240 may include an image recognition system 250. Image recognition system 250 may utilize machine learning to identify objects and gestures in images and video clips. The training may include obtaining a large dataset of labeled images or video clips that include the objects and/or gestures that are to be identified. Using techniques such as convolutional neural networks (CNNs), relevant features from the images are automatically extracted. A machine learning model (e.g., a deep learning model) is trained on the extracted features. Once trained, the model may be used to predict the presence of objects or gestures in new, unseen images and/or video clips. The images and/or video clips may include images of non-human animals exhibiting facial expressions, performing gestures, and/or other interpretable behaviors. Image recognition system 250 may further utilize Haar cascades for object detection. One or more embodiments may include training the Haar cascade classifier using a combination of positive samples and negative samples. The training process may include selecting the most relevant features and creating a cascade of classifiers.

The machine learning model array 240 may include, as an output, non-human informational output 260. The non-human informational output 260 may include audio output. The audio output may include species-specific audio waveforms such as clicks and songs for cetaceans, growling and/or barking sounds for canines, and so on. In an aquatic environment such as depicted in FIG. 1, the audio output may be provided by underwater speakers or other suitable transducers. The non-human informational output may include visual output, such as flashing lights, and/or patterns rendered and presented on an electronic display that is visible to the animals that are participating in the system and/or method for multimodal orchestration for human-animal-robot collaborative task execution.

The machine learning model array 240 may include, as an output, human-based informational output 270. The human-based informational output 270 may include visual information such as text and/or symbology. The human-based informational output 270 may include audio information. The audio information may include synthesized speech, tones, and/or other sounds to convey information identified by the machine learning model array 240. Referring again to the example depicted in FIG. 1, for investigation of the shipwreck 145, the non-human informational output 260 may include audio waveforms that may be interpreted by a whale 108 to swim to a location proximal to the shipwreck 145. The whale 108 may then output audio vocalizations in response to viewing the shipwreck. The output audio vocalizations may be translated by the system 200 to human-based informational output 270 for interpretations by humans. In this way, the non-human informational output 260 and the human informational output 270 may work in tandem to enable human-animal-robot collaborative task execution.

In one or more embodiments, system 200 extends beyond a single-agent paradigm by supporting networked, multi-agent orchestration across multiple human users, non-human animals, and autonomous robotic devices. The system 200 facilitates distributed decision-making, allowing each participant to act semi-autonomously while remaining interconnected through real-time data synchronization and task coordination mechanisms.

Each non-human participant (e.g., working dogs, marine mammals, robotic drones) may be equipped with edge computing capabilities, enabling on-device signal processing and local decision-making. For example, a canine’s EEG sensor may continuously monitor neural patterns for anomalies indicative of stress or target recognition, allowing immediate, localized intervention before escalating findings to a human handler or autonomous controller. The real-time inference module running on each wearable device enables the system to pre-filter raw neural signals, extracting only the most relevant features for transmission, thereby reducing bandwidth usage and improving system responsiveness.

To further enhance multi-agent adaptability, system 200 may implement federated learning, allowing multiple edge devices to perform localized model training while periodically synchronizing learned parameters with the central system. This approach prevents raw neural data from being centralized, ensuring that each participant continuously benefits from system-wide learning updates while maintaining privacy and reducing computational overhead.

By integrating distributed intelligence, real-time signal filtering, and federated model training, system 200 enhances operational efficiency, allowing a network of human and non-human agents to collaborate seamlessly across dynamic and unpredictable environments.

In one or more embodiments, the system 200 may include a machine-learning architecture configured to process time-series EEG data using a hybrid model that combines convolutional neural networks (CNNs) with recurrent neural networks (RNNs), such as long short-term memory (LSTM) networks or gated recurrent units (GRUs). The hybrid architecture enables the model to capture both spatial dependencies within neural signals and temporal patterns associated with cognitive and emotional states. The machine-learning system may be trained on a dataset comprising annotated brainwave recordings collected from multiple species, including dogs, cats, horses, and aquatic mammals such as dolphins. These datasets may be generated under controlled conditions and labeled with neural patterns corresponding to specific emotional states, such as excitement, stress, or calmness.

In one or more embodiments, the dataset may be supplemented with synthetically generated data using a generative adversarial network (GAN). This technique may be employed to augment underrepresented neural patterns in the training dataset, ensuring that the model achieves greater accuracy across species with differing signal distributions. The training pipeline may include preprocessing steps such as noise filtering, normalization, and segmentation of EEG signals into fixed-length time windows. To account for interspecies variation, each dataset entry may be associated with a species-specific embedding vector, enabling the model to learn species-dependent neural characteristics while preserving generalizable features for emotion classification.

In one or more embodiments, hyperparameter tuning techniques may be applied to optimize model performance across different species. Techniques such as Bayesian optimization or structured search may be employed to refine parameters, including network depth, activation functions, and learning rates, to improve accuracy and reduce overfitting. To further enhance training stability, early stopping criteria based on validation loss and cross-validation across species-specific data partitions may be implemented. The machine-learning system may also incorporate a multi-head attention mechanism, which allows the model to dynamically focus on the most informative neural signal regions, thereby mitigating the variability of EEG signals across species. By jointly optimizing for multiple tasks, such as classifying emotional states and reconstructing input signals, the system may enhance robustness and generalization across diverse neural inputs.

In one or more embodiments, the translation processing unit 220 may be implemented using a sequence-to-sequence model trained to map neural signal patterns to structured human language outputs and vice versa. The translation processing unit may receive extracted neural features from the machine-learning system and process them through an encoder-decoder architecture. The encoder may generate a latent representation that captures the essential characteristics of the animal’s cognitive or emotional state, while the decoder may transform this representation into a human-interpretable output, such as a textual description (“The dog is excited”) or a command (“Sit” or “Come here”). This transformation may be further optimized through an attention mechanism, allowing the model to selectively prioritize the most relevant portions of the neural signal when generating the final output.

In one or more embodiments, a training dataset may be constructed by pairing neural signal recordings with corresponding behavioral observations and expert annotations, effectively serving as a parallel corpus for training the translation model. Neural recordings captured during a non-human animal’s response to a stimulus or command may be aligned with the observed behavior and its associated emotional label, providing structured input-output mappings for supervised learning. The system may be trained using iterative refinement techniques, such as initially employing teacher forcing during early training stages and later transitioning to a scheduled sampling framework, allowing the model to improve its ability to generate responses independently.

To ensure adaptability across species, the translation processing unit may be fine-tuned on species-specific subsets of data, allowing it to learn the subtle variations in communication patterns while maintaining a unified mapping framework applicable across different animals. This adaptive learning approach ensures that the system may not only translate between neural signals and human language but also accurately infer the underlying intent and emotional state, thereby enhancing interspecies communication.

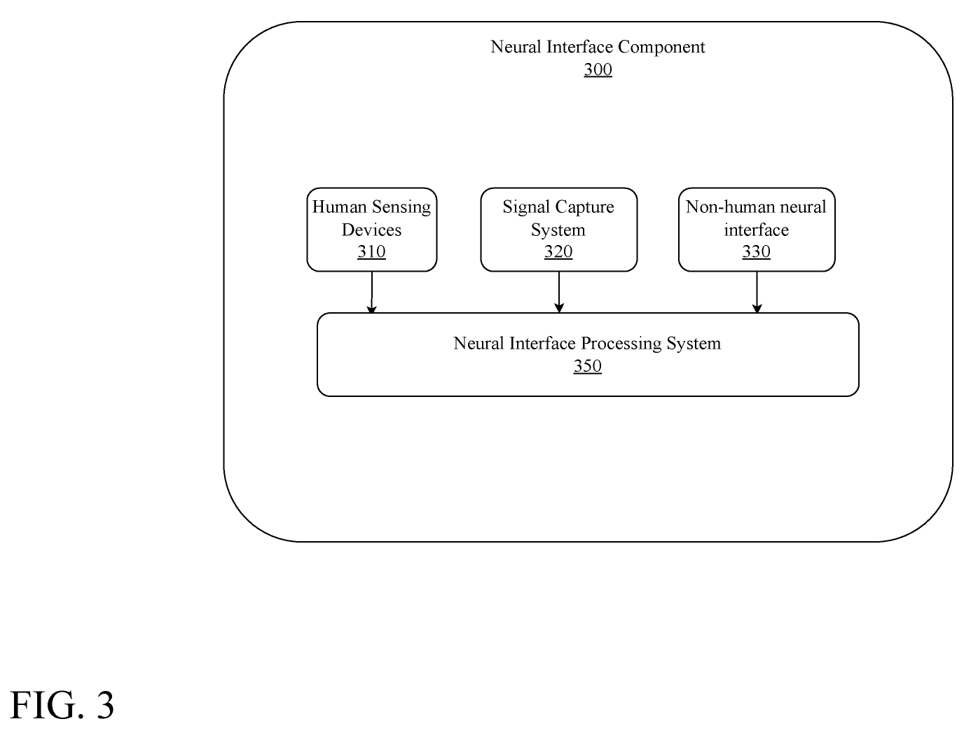

FIG. 3 is a block diagram illustrating details of a neural interface component, in accordance with one or more embodiments. Neural interface component 300 may be similar to neural interface component 210 of FIG. 2 and may include one or more human sensing devices 310. The human sensing devices 310 may include wearable sensors, such as pulse sensors, brainwave monitors, and the like. The human sensing devices 310 may further include cameras, microphones, and/or other sensors for obtaining cognitive state information from a human. The data received by the human sensing devices may be used with NLP module 244 to extract additional context and sentiment from humans participating in human-animal-robot collaborative task execution.

Neural interface component 300 may include a signal capture system 320. The signal capture system 320 may include one or more sensors for capture of neural and physiological signals without distress, adjusted for the physical characteristics of animals, such as cetaceans. In embodiments, neural sensors are embedded within wearable and/or attachable devices suited for underwater and open-ocean deployment. In embodiments, the neural interface includes components tailored to the species’ specific anatomy, such as the head or dorsal regions in whales, and made from materials that ensure durability and comfort even in the deep-sea environment.

Neural interface component 300 may include a non-human neural interface 330. The non-human neural interface 330 may include non-invasive sensors that may detect and measure animal brain activity without causing discomfort. These sensors capture neural signals associated with emotions, intentions, and responses to stimuli. The non-human neural interface 330 may further include implanted sensors. Embodiments may include surgically implanting sensor probes inside an animal’s brain. In embodiments, this technique may be used in place of a non-invasive sensor package, and may yield additional control and benefits that include the ability to record specific thoughts, evaluate mental state, and other aspects outside of the direct intent to communicate. This also enables capturing the animal’s sensory data such as vision and scent. This allows human/animals to not only communicate freely, but opens additional options for working animals. For example, a dog must pass extensive training before it may sniff drugs. With disclosed embodiments, the training may be drastically shortened by reading the animal’s brain patterns directly and identifying targeted substances via this data. As part of the training, the non-human informational output 260 (of FIG. 2) may produce a signal to cause happiness or the notion of correctness as a positive reward and drastically speed training times and animal willingness.

The output of the human sensing devices 310, signal capture system 320, and non-human neural interface 330 may be input to neural interface processing system 350. Neural interface processing system 350 may include an AI-based processing unit that analyzes and interprets vocalization data, behavioral cues, and environmental contexts to foster bidirectional communication between humans and non-human animals. Disclosed embodiments may be well-suited for applications ranging from enhancing human-animal interactions for conservation to advancing scientific understanding of animal languages, particularly in highly social and intelligent species such as sperm whales. Furthermore, disclosed embodiments may enable the use of environmental and behavioral metadata integration, as well as robust machine learning frameworks, allowing creation of an adaptable model for studying communication across various species, making disclosed embodiments adaptable to diverse animal communication needs beyond cetaceans, such as canines, primates, and other species.

In one or more embodiments, the neural interface component 300 may be augmented with expanded modalities of communication beyond neural signals. Such implementations may comprise a distributed sensor network deployed on or near the animal, potentially incorporating olfactory sensing modules capable of detecting volatile chemical compounds and specific scent cues, cardiac and physiological monitors that continuously capture heart rate and respiration patterns, body posture and motion detection systems utilizing inertial measurement units and computer vision-based pose estimation, and thermal imaging with additional environmental sensors to measure surface temperature and ambient conditions. The system may implement multimodal data fusion and cognitive state inference through a central computing device or distributed edge processors. In some implementations, the inference engine may be a multi-stream deep neural network where each sensor modality is processed by a dedicated sub-network, with outputs concatenated in fusion layers that jointly analyze combined features to produce probability distributions over possible cognitive or emotional states. This multimodal approach may enable accurate discernment of an animal’s state even in complex or ambiguous scenarios, such as distinguishing between excitement due to play versus excitement due to detecting a target odor by correlating neural patterns with supplementary sensor data.

The neural interface component illustrated in FIG. 3 represents one implementation approach, which may be informed by state-of-the-art developments in brainwave analysis. Current advanced EEG analysis increasingly utilizes deep learning techniques rather than relying solely on hand-crafted features. While traditional analysis employed Fourier bands and classical classifiers, contemporary approaches may use deep neural networks to learn complex spatial-temporal features directly from EEG data. In various embodiments, the neural interface may incorporate transformer-based models with self-attention mechanisms, which have demonstrated excellence in handling EEG noise and variability. These models may use attention to highlight important signal segments, improving interpretability and robustness to artifacts. In animal brainwave analysis specifically, the system may build upon pioneering studies in decoding neural signals from various species, adapting algorithms originally developed for human subjects to the unique neural signatures of non-human animals.

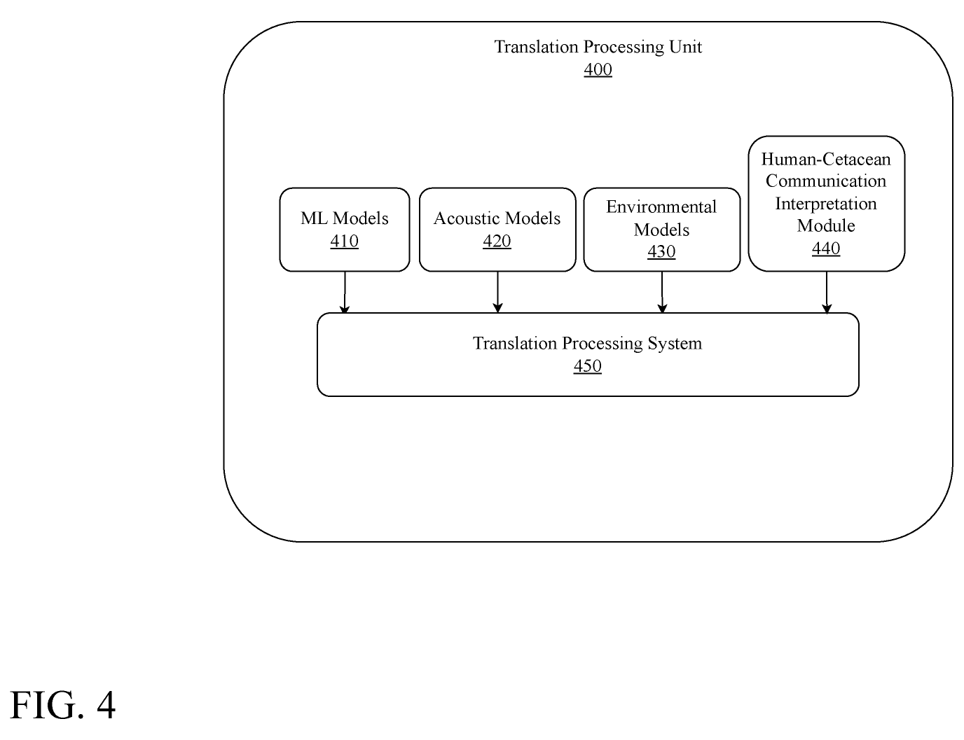

FIG. 4 is a block diagram illustrating details of a translation processing unit, in accordance with one or more embodiments. Translation processing unit 400 may be similar to translation processing unit 220 of FIG. 2 and may include one or more machine learning models 410. In embodiments, the machine learning models 410 may be trained to identify patterns in vocalization, such as codas, tempo, rhythm, ornamentation, and contextual variations like rubato. This training enables disclosed embodiments to decode structured and nuanced elements of cetacean communication, potentially revealing hierarchical or associative structures similar to human language. The translation processing unit 400 may include one or more acoustic models 420. The acoustic models 420 may enable replication of fricative production. This includes adjusting tongue position, airflow velocity, and constriction points. The translation processing unit 400 may further include one or more environmental models 430. The environmental models 430 may include probabilistic models to capture the uncertainty and variability inherent in fricative sound production and perception. The translation processing unit 400 may further include a human-cetacean communication interpretation module 440. In embodiments, the human-cetacean communication interpretation module 440 may enable mappings between humans and cetaceans. As an example, the clicks, songs, and codas of whales may be processed via machine learning, and mapped to human sentiments, such as danger, affection, joy, aggression, curiosity, and/or cooperation. In embodiments, the human sentiment may be derived from animal outputs, such as sounds made by animals, gestures made by animals, facial expressions made by animals, and so on. The danger may be represented by high-pitched whistles, rapid clicks, and/or abrupt calls. Affection may be represented by soft clicks, whistles, and/or low-pitched moans. Joy may be represented by rapid clicks, whistles, and/or varied, upbeat vocalizations. Aggression may be represented by loud, forceful vocalizations, grunts, or low-frequency rumbles. Curiosity may be represented by short bursts of clicks. Cooperation may be represented by repetitive clicking patterns. In general, cetacean sounds may be catalogued and correlated to a behavioral context.

The translation processing system 450 receives input from the ML models 410, acoustic models 420, environmental models 430, and human-cetacean communication interpretation module 440. The translation processing system 450 may be configured to convert animal neural patterns into human-comprehensible language, conveying the emotional state, intentions, and/or needs of the animal. The translation processing system 450 may further be configured to translate human speech into neural signals or cues that are meaningful and understandable to animals, allowing the animals to comprehend specific commands or sentiments directly. In embodiments, the translation processing system 450 may be configured to produce spoken translations of animal communications, thereby allowing humans to understand an animal’s emotions or needs. For instance, the system might translate a dog’s neural signals into phrases like “I am hungry” or “I’m feeling anxious.”

In one or more embodiments, the translation processing unit 400 may incorporate a direct neural stimulation module that not only reads but also writes to the animal’s brain in a controlled, non-invasive manner. A wearable interface, such as an advanced EEG headband integrated with stimulation electrodes, may deliver calibrated electrical currents via transcranial electrical stimulation and focused ultrasonic pulses via ultrasonic neuromodulation to targeted cortical regions. The stimulation parameters—including current intensity, waveform shape, frequency, and duration—may be dynamically adjustable based on real-time analysis of neurophysiological data. The system may implement an advanced neural stimulus coding scheme translating human commands into specific spatiotemporal patterns of neural stimulation, potentially using evoked sensory cues, direct motor modulation, or reward center stimulation approaches. In certain implementations, the control software may integrate stimulation modules within a closed-loop feedback framework that monitors resultant neural activity through concurrent EEG readings and physiological telemetry, enabling the processor to confirm that the intended neural signature has been evoked and to adjust stimulation parameters dynamically. The translation processing system 450 may utilize this bidirectional capability to establish more robust communication channels between humans and animals, effectively “speaking” in the animal’s neural language by inducing predetermined sensations or responses that serve as communication elements.

The translation processing unit shown in FIG. 4 may extend beyond conventional processing by incorporating neurosymbolic approaches that combine the pattern recognition capabilities of neural networks with the reasoning strengths of symbolic AI. In certain embodiments, this unit may decode an animal’s signal using a neural model and then apply symbolic logic or knowledge to interpret it contextually. This approach may address limitations of purely neural models by incorporating a knowledge base of animal behavior and logical rules to achieve deeper understanding. For example, if an animal displays certain neural patterns, the system might not only classify them as “excitement” but could further reason that the excitement likely relates to feeding time based on contextual information and rules. By decoupling abstract reasoning from raw signal processing, the translation unit may enhance adaptability across tasks and species, potentially allowing the same core model to be used for multiple species with species-specific symbolic modules handling interpretation.

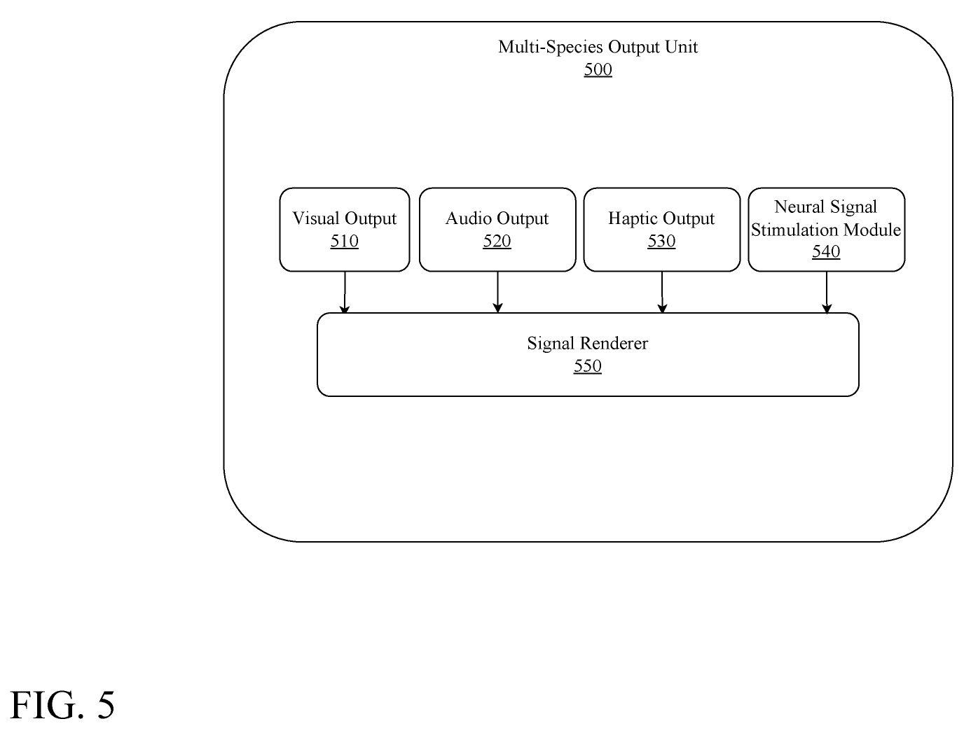

FIG. 5 is a block diagram illustrating details of a multi-species output unit 500, in accordance with one or more embodiments. The multi-species output unit is an innovative computerized system designed to facilitate communication between humans and animals by integrating multiple sensory modalities for output. The visual output subsystem 510 processes and assembles video and image data for output on an electronic display. The visual output is tailored to the perceptual capabilities of the target species, ensuring that animals or humans may interpret the visual signals effectively. The audio output subsystem 520 may be configured to generate audio waveforms that may be output through speakers or other audio devices. The audio output may include species-specific sounds, such as vocalizations, frequencies, or tones, enabling communication in a form recognizable by the intended animal. The haptic output subsystem 530 generates and modulates signals to drive vibratory devices, creating tactile sensations that may be detected via wearable sensors or directly on the animal’s skin. The haptic feedback may convey information such as alerts, directions, or emotional cues. The neural signal stimulation module 540 may include electrodes capable of monitoring and interacting with brainwaves of an animal. It may record neural activity and deliver carefully modulated stimulation to influence or reinforce specific neural patterns. This capability offers potential for advanced applications, such as training, behavior modification, or facilitating direct neural communication. The signal renderer module 550 serves as the central decision-making unit, determining the most appropriate output device for each signal. It integrates the outputs from the visual, audio, haptic, and neural modules and ensures that the signals are delivered in a coherent and species-appropriate manner. In embodiments, the multi-species output unit 500 may be integrated into, or communicatively coupled with, non-human informational output 260 and/or human-based informational output 270.

In one or more embodiments, the multi-species output unit 500 may implement an augmented reality interface integrated with a high-speed communication network bridging the animal’s wearable sensor array and the handler’s head-mounted display or mobile device. The animal-borne unit may transmit data via ultra-low latency protocols to an edge computing device that employs sensor fusion algorithms to generate normalized state vectors corresponding to the animal’s mental and emotional states. The AR unit itself may be equipped with GPU-accelerated rendering and adaptive calibration that dynamically adjusts overlay parameters, ensuring that virtual indicators—such as colored auras, iconographic symbols, directional arrows, and textual cues—remain spatially registered to the animal’s image and contextually aligned with the environment. This embodiment may extend the output unit’s functionality to include bi-directional communication that enables both visualization of the animal’s state and projection of guided commands to the animal. In certain implementations, the AR system may incorporate a semantic mapping module that constructs a multi-dimensional environmental model by integrating visual, auditory, and sensor-derived data, identifying and cataloging salient features of the operational environment and anchoring them within the AR overlay framework. The visual output subsystem 510, audio output subsystem 520, haptic output subsystem 530, and neural signal stimulation module 540 may all be coordinated through this enhanced AR interface to provide comprehensive situational awareness and coordinated response strategies.

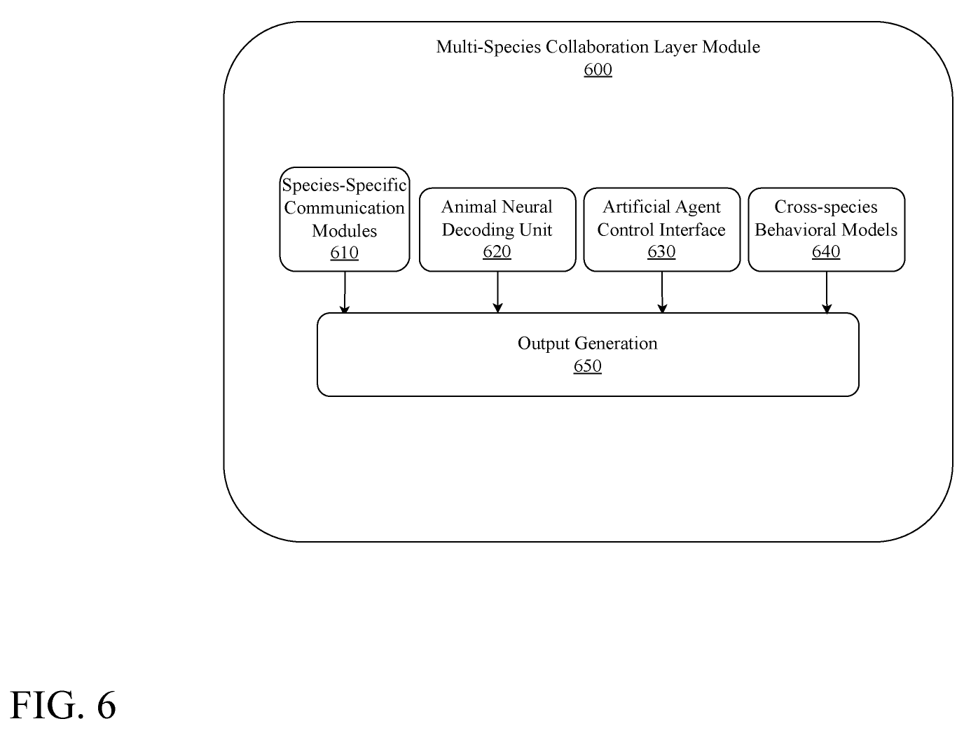

FIG. 6 is a block diagram illustrating details of a multi-species collaboration layer module 600, in accordance with one or more embodiments. The Multispecies Collaboration Layer (MCL) builds on the foundational capabilities of neural interfaces, multimodal sensory processing, and language modeling techniques, extending them to facilitate purposeful, synchronized interaction across species or between animals and machines. Its key functions are to understand interspecies “vocabularies,” align goals, and orchestrate collaborative tasks. The MCL module 600 may include one or more species-specific communication modules 610. In embodiments, each participating species (e.g., dogs, elephants) or artificial agent (e.g., a drone) has its own communication interface and representation layer.

The MCL module 600 may further include an animal neural decoding unit 620. The animal neural decoding unit 620 may be configured to extract interpretable “meaning vectors” from the animal’s neural signals and observed behavior. As an example, for a dog, neural patterns plus posture/vocalization cues may produce a “conceptual state embedding” representing intentions and emotional states. The MCL module 600 may further include an artificial agent control interface 630. For a drone, sensor data (LIDAR, camera, GPS) and command frameworks are translated by the artificial agent control interface into abstract action representations (e.g., “search pattern initiated,” “altitude adjustment needed”). The MCL may further include one or more cross-species behavioral models 640. These models may utilize a library of known behavioral cues and tasks common to various species (e.g., “move towards scent,” “alert upon detection of target”) to produce standardized action and intention descriptors.

The MCL module 600 further includes an output generation module 650 that receives input from the species-specific communication modules 610, animal neural decoding unit 620, artificial agent control interface 630, and/or cross-species behavioral models 640. The output generation module 650 then generates an appropriate output signal, which may include a video signal, audio signal, haptic signal, and/or other bioelectrical signal for conveying sentiment and/or meaning among humans and non-human animals. The output generation module 650 may output data in a wide variety of digital and/or analog formats, including pulse code modulated (PCM) audio, raw video formats, compressed video formats, and/or other suitable formats.

In embodiments, the Multispecies Collaboration Layer may be configured to provide a unified, context-driven platform enabling animals of different species, as well as robots and/or drones, to collaborate effectively on shared tasks. By creating and refining interspecies dictionaries, using ML models to align intentions, and carefully timing and routing these concepts through a shared task representation space, the MCL module 600 may enable synchronized, purposeful action. This may profoundly enhance capabilities in wildlife conservation, service support, and environments where diverse species and agents work in concert to achieve common goals. In embodiments, the MCL module 600 may be integrated with, or communicatively coupled to, system 200 of FIG. 2.

In one or more embodiments, the Multi-Species Collaboration Layer (MSCL) 600 facilitates multi-agent coordination between human users, non-human animals, and robotic systems by dynamically routing task-relevant data across participants. The MSCL 600 operates as a real-time decision hub, aggregating brainwave data, sensor outputs, and behavioral signals to optimize collective intelligence and coordination.

To enable multi-agent reinforcement learning (MARL), the MSCL 600 assigns localized reward functions to each participant based on performance metrics, such as a canine’s ability to locate a target or a robotic unit’s mapping efficiency. As participants complete tasks, their individual learning experiences contribute to a shared global policy, ensuring that knowledge is distributed across the system. For example, if a first canine identifies a contraband scent at 92 % confidence, this detection pattern may be propagated to all other canine participants via federated model updates, improving search efficiency without requiring centralized data exchange.

Additionally, the MSCL 600 employs policy-based message routing, ensuring that only relevant data streams are forwarded to individual participants. If a stress alert is detected from a first non-human animal (e.g., Dog #3), the MSCL 600 may prioritize notification to nearby drones or handlers, while filtering non-essential alerts for other distant participants. The MSCL 600 also integrates graph-based orchestration, in which all participants (human, animal, or robotic) are represented as nodes in a multi-agent collaboration graph, with adaptive link weights based on task priority, proximity, and cognitive load. The system continuously reallocates tasks based on real-time updates, allowing agents to dynamically adjust roles and responsibilities based on changing environmental conditions.

By incorporating these multi-agent coordination techniques, the MSCL 600 enables real-time cross-species intelligence sharing, improves task efficiency, and ensures adaptive decision-making in complex operational environments, such as search and rescue, security operations, and wildlife monitoring.

In one or more embodiments, the multi-species output and collaboration module 600 may be configured as a unified system that dynamically translates processed neural data into species-specific output signals while also synchronizing tasks among human, non-human animal, and robotic participants. In some implementations, the module may generate multi-modal communication outputs that align with the sensory and cognitive capabilities of each species.

For example, upon detecting a neural pattern indicative of a specific emotional or cognitive state in a non-human animal—such as a canine exhibiting excitement—the system may trigger a haptic feedback module on a wearable collar, generating a vibration pattern that the canine has been conditioned to recognize as a behavioral cue. Simultaneously, the system may generate an audio output signal that is frequency-optimized for canine auditory perception, reinforcing the behavioral instruction through redundant sensory modalities. By integrating multiple output channels, the system may enhance the clarity and reliability of command transmission, ensuring that species-specific cues are received and correctly interpreted by the intended recipient.

In one or more embodiments, the system may be configured to support collaborative operations involving humans, non-human animals, and autonomous robotic agents, such as in a search-and-rescue mission within a disaster zone. The multi-species collaboration layer may continuously process neural, environmental, and sensor-derived data from multiple sources, including wearable EEG sensors on working animals, drone-mounted cameras, and environmental detection units. When the system detects that a non-human animal, such as a search-and-rescue canine, has identified a potential victim, the multi-species output module may generate concurrent, modality-specific output signals for each collaborating entity.

For example, upon detecting a positive identification, the system may dispatch a visual indicator to a robotic drone, causing a highlighted marker to appear on a digital map interface, while also generating an auditory cue through a portable speaker to alert nearby human responders. This synchronized multi-agent communication strategy ensures that all participants—human, animal, and robotic—receive situationally relevant information in formats tailored to their respective sensory modalities, thereby optimizing real-time coordination and response efficiency in dynamic or high-stakes environments.

In one or more embodiments, the multi-species collaboration layer module 600 may be implemented as a Context-Aware Recursive Interaction Module (CARIM) that enables detailed scene understanding and bidirectional communication among humans, non-human animals, and robotic agents. Such implementations may integrate multi-modal data—including neural signals, visual data, text descriptions, and environmental sensor information—into a unified, continuously updated context model via neurosymbolic processing and hypergraph-based message passing. The module may utilize transformer-based encoders with multi-head attention to project each modality’s features into a unified embedding space, yielding modality-specific representations. A hypergraph may be constructed wherein each vertex corresponds to an individual representation, with similar vertices aggregated to capture semantic associations among modalities. The module may employ a Recursive Iterative Search mechanism that continuously queries the hypergraph for analogous contexts and patterns, evaluating multiple potential future states of the shared context vector. This implementation may leverage multi-modal recursive learning, neurosymbolic fusion for explainable reasoning, enable bidirectional real-time communication, and provide scalability and interoperability across diverse operational environments. Central to such implementations may be a shared context vector representing fused semantic information that serves as input to a neurosymbolic reasoning engine, which integrates symbolic rules with neural outputs to produce interpretable, context-aware translations and commands.

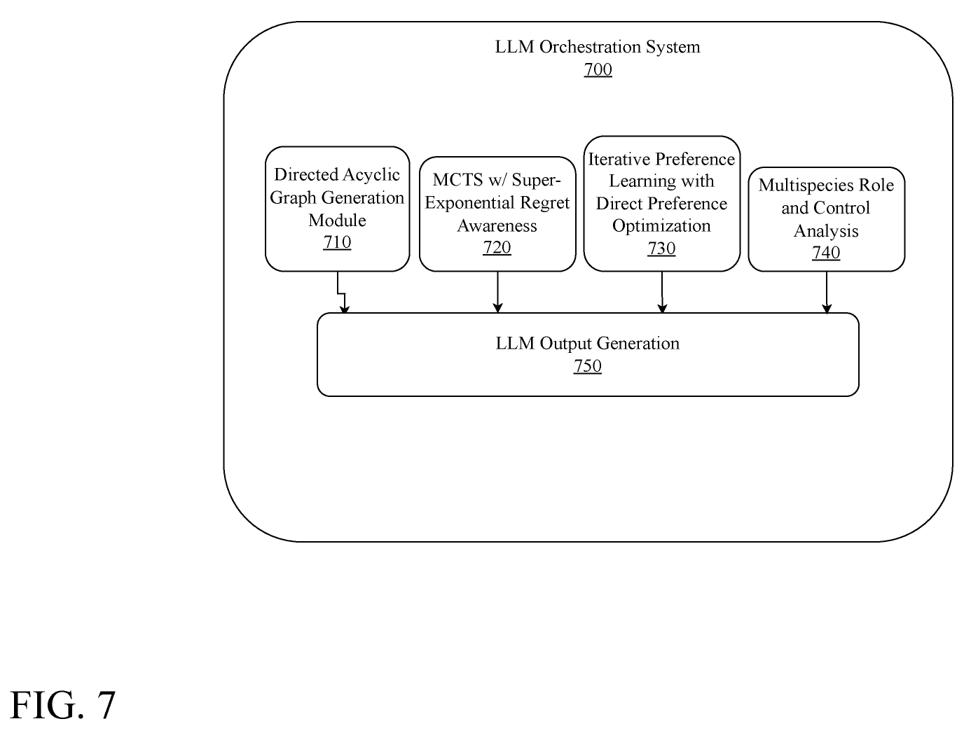

FIG. 7 is a block diagram illustrating details of a large language model (LLM) orchestration system 700, in accordance with one or more embodiments. In embodiments, LLM orchestration system 700 may be integrated with, or communicatively coupled to, LLM 242 of FIG. 2. LLM orchestration system 700 may include directed acyclic graph (DAG) generation module 710. The DAG generation module 710 may create a DAG representing complex workflows in which nodes are reasoning steps, and edges represent transitions from one partial solution to another. In some embodiments, each node encodes the current state of the environment, such as position and behavior of animals, humans’ textual instructions, and/or robot sensor readings. The DAG’s expansions may correspond to MCTS-like searches over possible reasoning paths, guided by previously described methods (e.g., embedding caches, semantic KGs, preference learning). Embodiments may include generating a directed acyclic graph to represent a plurality of reasoning steps corresponding to the multispecies coordinated task execution.

The LLM orchestration system 700 may include MCTS w/ Super Exponential Regret Awareness module 720. In this context, “super-exponential regret” refers to the phenomenon where certain algorithms, specifically the Upper Confidence bounds applied to Trees (UCT) and its variants like AlphaGo’s Monte Carlo Tree Search (MCTS), may experience regret that grows at a super-exponential rate under specific conditions. Regret, in this setting, measures the difference between the actual performance of the algorithm and the optimal performance it could have achieved. This module 720 may adjust model parameters to reduce or minimize regret, thereby improving performance. The adjustments made by module 720 may include modifying exploration-exploitation tradeoffs (e.g., fine-tuning of exploration constants in UCT).

The LLM orchestration system 700 may include iterative preference learning with direct preference optimization module 730. In embodiments, each node’s expansions produce step-level preference data: which partial expansions yield better outcomes (improved translation quality, correct interpretation of animal signals). After collecting these preferences (through MCTS expansions and intermediate verification from debate steps), module 730 may apply Direct Preference Optimization (DPO) to refine the LLM’s underlying policy. Over multiple cycles, on-policy sampled data enable the LLM’s decision-making to improve at picking high-value expansions from the start. This reduces reliance on brute-force exploration and counters the conditions leading to super-exponential regret.

The LLM orchestration system 700 may include multispecies role and control analysis module 740. In embodiments, module 740 may model agents as having different influence roles, which may dynamically encourage certain agents to lead expansions in known-productive directions. Moreover, module 740 may be configured to let other agents anchor or block suspicious expansions (such as an octopus punching unhelpful fish), which are translated into immediate pruning of subgraphs in the reasoning DAG.

The LLM orchestration system 700 may include an LLM output generation module 750 which receives as input, outputs from the directed acyclic graph generation module 710, MCTS with Super Exponential Regret Awareness module 720, include iterative preference learning with direct preference optimization module 730, and/or multispecies role and control analysis module 740. The output from the LLM output generation module 750 may include information for human consumption, such as textual information, knowledge-based outputs, and/or structured data. The output from the LLM output generation module 750 may include information for robot consumption, such as commands, sensor data, and/or other command and control information. The output from the LLM output generation module 750 may include information for animal consumption, such as audio waveforms intended for interpretation by animals, such as tones and/or click patterns for cetaceans, tones and/or sounds for canines, and so on. Other signals for representation in visual and/or haptic domains may also be output by LLM output generation module 750 in some embodiments.

In one or more embodiments, LLM orchestration system 700 is enhanced with digital twin modeling, allowing each participant—whether human, non-human animal, or robotic device—to maintain a virtual AI representation of its cognitive state, learned behaviors, and task preferences. These digital twins function as adaptive learning agents, continuously updating their models based on historical interactions, task performance, and evolving situational demands.

System 700 may further integrate federated learning techniques, such as Federated Averaging (FedAvg), to allow each device (e.g., EEG collars, robotic vision modules, or human communication interfaces) to execute localized training on real-world interactions, refining decision-making without requiring direct access to other participants’ raw neural or sensory data. This approach not only enhances privacy and security but also improves data efficiency, ensuring that only essential knowledge updates are shared across the network.

Additionally, multi-agent negotiation protocols may be employed, wherein digital twins autonomously exchange contextual insights, coordinate training schedules, and allocate tasks based on real-time environmental conditions and operational priorities. For example, if a digital twin controlling a search-and-rescue canine detects an elevated stress pattern, it may automatically negotiate with a drone-based digital twin to deploy visual support and telemetry assistance in the affected area.

By integrating digital twin architectures, federated learning, and decentralized negotiation mechanisms, system 700 enables a scalable, multi-agent intelligence framework, ensuring that human-animal-robot interactions continuously adapt and optimize based on collective system-wide experiences.

In one or more embodiments, the LLM orchestration system 700 may expand its intelligence into an inter-species collaborative AI framework leveraging federated learning to continuously improve communication models across diverse animal types. In such implementations, each wearable device may operate as a client in a federated learning network, computing local model updates that are securely aggregated at a centralized server to refine the global model while preserving data privacy. The system may support multi-species data aggregation and semantic alignment through a unified representation layer that extracts features common across species before diverging into species-specific sub-modules. Cross-species model sharing may propagate improvements across the network, potentially allowing devices monitoring biologically similar species to benefit from enhanced detection capabilities even with limited direct training data. Additionally, some implementations may integrate a dynamic meta-prompt optimization module leveraging federated adversarial bandit learning. Each animal-human interface may include a local meta-prompt engine comprising task descriptions, meta-instructions, and curated exemplar sequences transformed into high-dimensional embeddings. The local module may evaluate candidate meta-prompts using exponential weighting schemes inspired by adversarial bandit algorithms, with exemplar selection components dynamically refining sequences via analogous mechanisms. This approach may enhance the directed acyclic graph generation module 710, MCTS module 720, preference learning module 730, and multispecies role analysis module 740 by providing continuously optimized communication frameworks that adapt to each specific animal-human interaction context while benefiting from collective learning across the entire network.

The LLM orchestration system depicted in FIG. 7 may be implemented with advanced neurosymbolic components to push beyond current limitations. In some embodiments, the system may employ a neurosymbolic translator engine that continuously learns from interactions, building an expanding knowledge graph linking neural patterns to outcomes and refining its reasoning over time. This approach could offer advantages in explainability and trust, as the symbolic components may trace logical rules or reference known facts to explain translations. The LLM system may implement bidirectional neurosymbolic communication, potentially using a common semantic representation internally—essentially a “Universal Interspecies Language” of concepts. In such implementations, neural decoders might map human and animal communications into this semantic space, perform symbolic reasoning there, and then map back out to each species. Certain embodiments may incorporate reinforcement learning to allow the AI to learn through interaction rather than just offline data, enabling the system to try new communicative acts and learn from responses, similar to how humans naturally acquire language skills.

In various implementations, the neural interface component of FIG. 3, the translation processing unit of FIG. 4, and the LLM orchestration system of FIG. 7 may work in concert to create a comprehensive system that not only translates with high accuracy but also contextually understands the interspecies dialogue. Such a system might continuously learn and reason to improve mutual understanding between humans, animals, and mediating robots, potentially enabling communication that approaches the natural feel of human-to-human interaction.

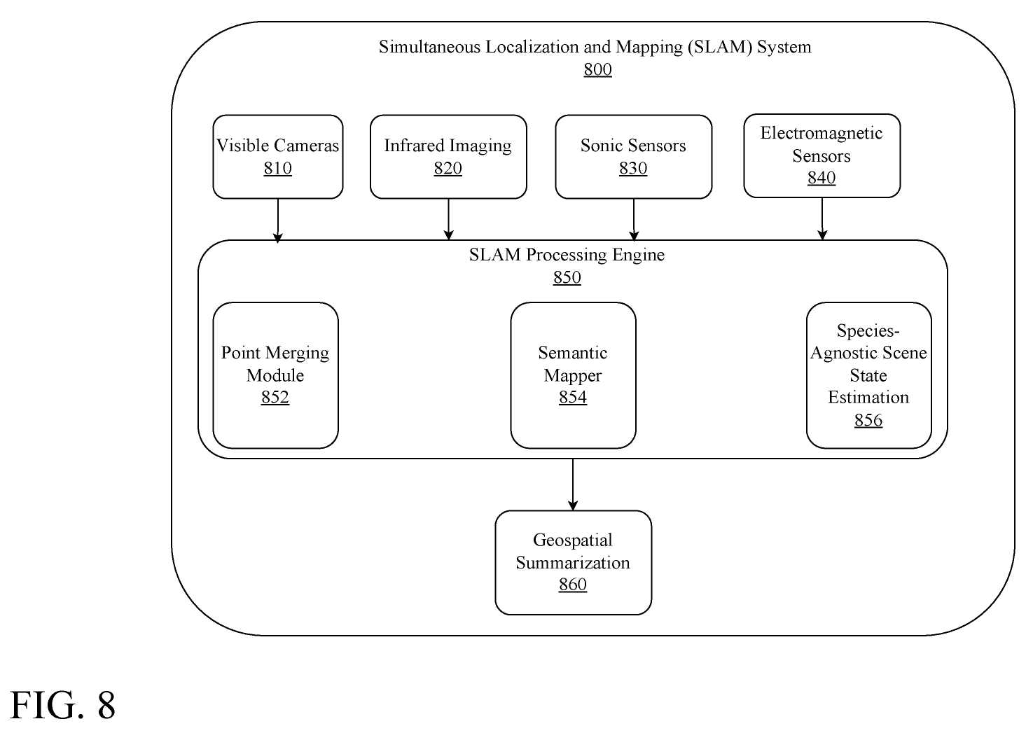

FIG. 8 is a block diagram illustrating details of a Simultaneous Localization and Mapping (SLAM) system 800, in accordance with one or more embodiments. In embodiments, SLAM system 800 may be integrated with, or communicatively coupled to, system 200 for multimodal orchestration for human-animal-robot collaborative task execution of FIG. 2. System 800 may include visible cameras 810, infrared imaging sensors 820, sonic sensors 830, and/or electromagnetic sensors 840. The visible cameras 810 may be configured to detect light in the visible spectrum (roughly 400–700 nanometers), which is the range of light perceptible to the human eye, and capture colors and details as humans see them, relying on external light sources (e.g., sunlight or artificial lighting) to illuminate a scene. The visible cameras may include wide-angle cameras, telephoto cameras, and so on. The infrared imaging sensors 820 may be configured to detect light in the infrared spectrum (beyond 700 nanometers), which is invisible to the human eye. In some embodiments, the infrared imaging sensors 820 may include thermal cameras that may capture emitted heat radiation from objects, even in complete darkness, without requiring external illumination. The sonic sensors 830 may include microphones and/or hydrophones. The microphones may include dynamic microphones, condenser microphones, electret microphones, and/or other suitable types of microphones. The hydrophones may include piezoelectric hydrophones that use piezoelectric materials to detect pressure changes in water caused by sound waves. The hydrophones may include vector sensors that measure both sound pressure and particle motion within water. The hydrophones may include a hydrophone array that includes multiple hydrophones arranged in a specific geometry to detect sound from multiple directions.

The electromagnetic sensors 840 may be configured to detect and measure electromagnetic fields or properties, such as electrical conductivity, magnetic fields, and electromagnetic radiation. The electromagnetic sensors 840 may include fluxgate magnetometers, suitable for detecting magnetic anomalies from seafloor rocks, or identifying metallic objects like shipwrecks or submarines. The electromagnetic sensors 840 may include proton precession magnetometers that may measure the magnetic field based on the precession of protons in water or a fluid. In one or more embodiments, the electromagnetic sensors may include optically pumped magnetometers, electric field detectors, capacitive sensors, electromagnetic induction sensors, and/or other suitable types of electromagnetic sensors.

The inputs from visible cameras 810, infrared imaging sensors 820, sonic sensors 830, and electromagnetic sensors 840 may be input to SLAM processing engine 850. SLAM processing engine 850 may include a point merging module 852. In embodiments, the point merging module 852 may include functions and instructions for combining multiple data points that correspond to the same real-world feature. This helps refine the map, reduce noise, and improve localization accuracy. SLAM processing engine 850 may include a semantic mapper 854. In embodiments, the semantic mapper 854 may include functions and instructions for enabling humans to interpret animal emotional states or intentions through augmented reality interfaces linked to embeddings and semantic mapping. The semantic mapper 854 may further include a Semantic Alignment Agent that may refine cross-domain mappings accordingly. Moreover, SLAM processing engine 850 may further include a species-agnostic scene state estimation module 856. In embodiments, the species-agnostic scene state estimation module 856 may include functions and instructions for utilizing data from visible light cameras to determine color and depth of a scene, enabling a 3D reconstruction of an environment. The species-agnostic scene state estimation module 856 may further include functions and instructions for utilizing data from ultraviolet (UV) and/or hyperspectral sensors, which may provide benefits as some animal signals might be visible only in UV or certain spectral bands, revealing hidden patterns (like UV-reflective markings on fish or subtle changes in an octopus’s skin). The species-agnostic scene state estimation module 856 may further include functions and instructions for utilizing data from lidar scanners that produce high-resolution point clouds for both indoor and outdoor environments. In one or more embodiments, radar may complement lidar in poor visibility conditions. The species-agnostic scene state estimation module 856 may further include functions and instructions for utilizing data from sonic and/or acoustic sensors to capture vocalizations from a wide variety of animals. Embodiments may further utilize beamforming and/or signal processing in order to locate sound sources and/or identify distress calls, barks, or other meaningful vocal patterns in non-human animals.

The output of the SLAM processing engine 850 may include a geospatial summarization 860. The geospatial summarization 860 may include data that may be rendered and presented on an electronic display to show features such as a map panel that indicates animal locations, terrain features, and environmental conditions. One or more embodiments may further include icons representing animals that are updated in real-time, displaying status indicators such as color-coded stress levels, activity patterns, and the like. The data output of geospatial summarization 860 may include data in a variety of raster, vector, and/or other suitable formats.

In one or more embodiments, the system may incorporate a Simultaneous Localization and Mapping (SLAM) module configured to construct and update a real-time environmental map using multiple sensor modalities. The SLAM module may integrate data from a network of underwater sensors deployed on buoys, autonomous underwater vehicles, and seafloor detection devices. These sensors may include visible-light cameras, infrared sensors, hydrophones, and electromagnetic detectors, which collectively capture a diverse set of environmental inputs. The collected data streams may be processed by a centralized SLAM processing engine that employs sensor fusion algorithms to merge disparate data points, calibrate measurements through techniques such as point cloud merging and temporal alignment, and generate an accurate three-dimensional representation of the surrounding environment.

In one or more embodiments, the SLAM module may be configured to detect and classify both static features, such as underwater rock formations or shipwreck debris, and dynamic entities, such as marine life, which may be relevant for applications such as coordinated search-and-rescue operations. The sensor fusion step may involve compensating for variations in noise levels and update rates across different sensors to generate a coherent environmental representation. In some implementations, an Extended Kalman Filter (EKF) may be employed to merge visual data from cameras with sonar measurements from hydrophones. The EKF may predict system states based on sensor dynamics and iteratively refine state estimates using incoming measurements. Alternatively, in one or more embodiments, the SLAM module may utilize GraphSLAM techniques, in which sensor observations are represented as nodes in a graph, and the global map is optimized through iterative least-squares refinement. This approach may be particularly effective in complex underwater environments where sensor data is inherently noisy or sparse.

In an exemplary implementation involving a search-and-rescue operation in a complex underwater terrain, the SLAM module may continuously process data from high-resolution cameras and acoustic sensors mounted on a dolphin, in combination with hydrophone arrays deployed on buoys. The cameras may capture fine-grained visual details of the underwater environment, while the dolphin-based acoustic sensors and buoy-mounted hydrophones may detect marine life signatures or structural sounds from a wreck site. The system may further incorporate a semantic mapping component that overlays additional data layers, such as depth readings, temperature gradients, and object classifications, onto the base map. In some implementations, the system may employ LIDAR and sonar fusion techniques to achieve a high-fidelity reconstruction of the underwater terrain. The resulting fused map may be transmitted to human operators via a tablet interface displaying a real-time three-dimensional model, while also generating task-specific instructions for non-human animals, such as dolphins, that enable dynamic adjustments to their search patterns based on newly detected features.

By integrating SLAM-based environmental awareness with sensor fusion and multi-agent coordination, the system may enhance situational understanding and enable synchronized decision-making across human, animal, and robotic participants in dynamic and high-stakes operational environments.

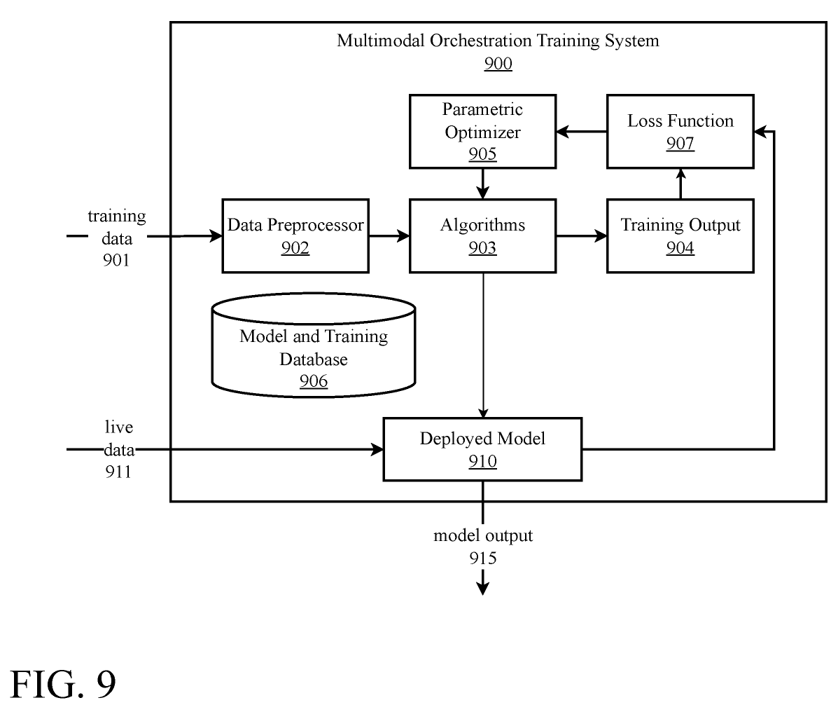

FIG. 9 is a block diagram illustrating an exemplary training system for tasks such as multimodal orchestration for human-animal-robot collaborative task execution and/or bilateral communication between humans and non-human animals, in accordance with one or more embodiments. In embodiments, system 900 may comprise a model training stage comprising a data preprocessor 902, one or more machine and/or deep learning algorithms 903, training output 904, a parametric optimizer 905, and a model deployment stage comprising a deployed and fully trained model 910 configured to perform tasks described herein such as enabling multimodal orchestration for human-animal-robot collaborative task execution. The system 900 may be used to train and deploy a plurality of AI subsystems in order to support the services provided by the system for multimodal orchestration for human-animal-robot collaborative task execution.

At the model training stage, a plurality of training data 901 may be received by the training system 900. Data preprocessor 902 may receive the input data (e.g., human feedback, human input data, animal input data, animal feedback, robot/sensor inputs, and the like) and perform various data preprocessing tasks on the input data to format the data for further processing. For example, data preprocessing may include, but is not limited to, tasks related to data cleansing, data deduplication, data normalization, data transformation, handling missing values, feature extraction and selection, mismatch handling, and/or the like. Data preprocessor 902 may also be configured to create a training dataset, a validation dataset, and/or a test set from the plurality of input data 901. For example, a training dataset may comprise 80 % of the preprocessed input data, the validation set 10 %, and the test dataset may comprise the remaining 10 % of the data. The preprocessed training dataset may be fed as input into one or more machine and/or deep learning algorithms 903 to train a predictive model for tasks that may include interspecies communication, geospatial mapping, and/or object monitoring and detection.

During model training, training output 904 is produced and used to measure the accuracy and usefulness of the predictive outputs. During this process a parametric optimizer 905 may be used to perform algorithmic tuning between model training iterations. Model parameters and hyperparameters may include, but are not limited to, bias, train-test split ratio, learning rate in optimization algorithms (e.g., gradient descent), choice of optimization algorithm (e.g., gradient descent, stochastic gradient descent, of Adam optimizer, etc.), choice of activation function in a neural network layer (e.g., Sigmoid, ReLu, Tanh, etc.), the choice of cost or loss function the model will use, number of hidden layers in a neural network, number of activation unites in each layer, the drop-out rate in a neural network, number of iterations (epochs) in training the model, number of clusters in a clustering task, kernel or filter size in convolutional layers, pooling size, batch size, the coefficients (or weights) of linear or logistic regression models, cluster centroids, and/or the like. Parameters and hyperparameters may be tuned and then applied to the next round of model training. In this way, the training stage provides a machine learning training loop.

In some implementations, various accuracy metrics may be used by the training system 900 to evaluate a model’s performance. Metrics may include, but are not limited to, word error rate (WER), word information loss, cetacean response times, predicted animal response compared with actual animal response, and normalization error rate, to name a few. In one embodiment, the system may utilize a loss function 907 to measure the system’s performance. The loss function 907 compares the training outputs with an expected output and determines how the algorithm needs to be changed in order to improve the quality of the model output. During the training stage, all outputs may be passed through the loss function 907 on a continuous loop until the algorithms 903 are in a position where they may effectively be incorporated into a deployed model 915.

The test dataset may be used to test the accuracy of the model outputs. If the training model is establishing correlations that satisfy a certain criterion such as but not limited to quality of the correlations and amount of restored lost data, then it may be moved to the model deployment stage as a fully trained and deployed model 910 in a production environment making predictions based on live input data 911 (e.g., user preferences, user feedback, user inputs). Further, model correlations and restorations made by deployed model may be used as feedback and applied to model training in the training stage, wherein the model is continuously learning over time using both training data and live data and predictions. A model and training database 906 is present and configured to store training/test datasets and developed models. Database 906 may also store previous versions of models.

According to some embodiments, the one or more machine and/or deep learning models may comprise any suitable algorithm known to those with skill in the art including, but not limited to, LLMs, generative transformers, transformers, supervised learning algorithms such as: regression (e.g., linear, polynomial, logistic, etc.), decision tree, random forest, k-nearest neighbor, support vector machines, Naïve-Bayes algorithm; unsupervised learning algorithms such as clustering algorithms, hidden Markov models, singular value decomposition, and/or the like. Alternatively, or additionally, algorithms 903 may comprise a deep learning algorithm such as neural networks (e.g., recurrent, convolutional, long short-term memory networks, etc.).

In some implementations, the training system 900 automatically generates standardized model scorecards for each model produced to provide rapid insights into the model and training data, maintain model provenance, and track performance over time. These model scorecards provide insights into model framework(s) used, training data, training data specifications such as chip size, stride, data splits, baseline hyperparameters, and other factors. Model scorecards may be stored in database(s) 906.

In one or more embodiments, the training system 900 may be configured to process and utilize a variety of training datasets for enabling neural interface-based communication among humans, animals, and robots. Such datasets may include, but are not limited to, publicly available animal communication datasets comprising vocalization libraries and neural recordings. For example, in various embodiments, the system may utilize large marine mammal vocalization datasets containing annotated sounds, canine bark audio collections, and/or publicly available brain-signal data from non-human subjects. In some embodiments, the system may access and process existing neural decoding datasets, such as those containing cortical recordings for brain-machine interface tasks. Furthermore, embodiments may leverage human BCI datasets, such as EEG motor imagery data, which may be repurposed or used for transfer learning, as decoding methods may overlap with animal signal processing.

In one or more embodiments, the training system 900 may supplement real data with synthetic and simulated datasets where actual data are limited. For instance, the system may incorporate generative models, such as GANs or diffusion models, to produce artificial EEG-like signals that augment training data. In some implementations, the system may generate 1-40 % accuracy improvements by adding synthetically generated EEG samples to train classifiers. Additionally, embodiments may simulate neural and behavioral signals in virtual environments, for example, creating reinforcement learning scenarios with simulated animal agents that generate signals, thereby allowing collection of paired signal/intent data. In some embodiments, the system may utilize multi-agent environments to generate synthetic interaction data for training translation models. Further embodiments may employ semi-synthetic approaches, such as training neural networks on synthetic data to track actual neuron activity in subjects.

In one or more embodiments, the training system 900 may utilize reinforcement learning environments to generate dynamic training data for communication. In such configurations, an AI agent, which may be embodied as a robot, may be trained to interpret animal signals by receiving rewards for correctly responding to those signals in a simulated task. Some implementations may employ an RL-based approach for autonomous robots or drones to interact with animals, effectively creating a closed-loop learning environment. In certain embodiments, the system may utilize an RL setup where a robot learns to respond to an animal’s EEG patterns or vocalizations through trial and error, gradually building a communication protocol. Such environments may produce sequences of signals, actions, and rewards that may be used to fine-tune communication models, particularly for agent-agent or agent-animal interaction.

In one or more embodiments, the training system 900 may incorporate neurosymbolic knowledge bases in addition to signal data. Such knowledge bases may store associations between animal behaviors or brainwave patterns and symbolic representations of emotions, intentions, or commands. In some implementations, ethological knowledge, such as specific EEG rhythm patterns linked to stress or attention in animals, may be encoded as rules or labeled examples. These may serve as additional training data for a model or as constraints during fine-tuning. Certain embodiments may integrate a knowledge graph, such as an ontology of animal communication signals, to help ground the learning in semantics. Additionally, neuro-symbolic AI approaches, which combine neural network learning with symbolic reasoning, may be implemented to enable a system to learn from raw data while also consulting logical rules or facts. Such knowledge bases could be built from various sources and augmented with animal-specific terms, potentially enabling models to not just fit patterns but also to reason about them, which could improve generalization and explainability of the communication system.

In one or more embodiments, the training system 900 may implement continuously adaptive learning modules designed to refine interspecies communication through dynamic machine learning techniques. Such implementations may begin with a personalized calibration phase where baseline neural signatures, physiological metrics, and behavioral responses are recorded and processed through pre-trained networks that are subsequently fine-tuned using on-device transfer learning protocols. The system may leverage reinforcement learning strategies to adjust interpretation and output parameters in real-time, with RL agents monitoring state vectors derived from sensor data and mapping these to communication outputs. The system may dynamically adjust internal parameters in response to contextual and temporal variations, potentially placing greater emphasis on robust EEG data over posture-based cues during periods of elevated ambient temperature or high physical activity. Meta-learning frameworks employing gradient-based optimization and Bayesian tuning of hyperparameters may facilitate this dynamic adjustment. Some versions may support co-adaptation paradigms wherein both the animal and the device learn to optimize their communication over time, reinforcing the production of distinct neural patterns through immediate, context-specific feedback. Such adaptive systems may be realized by integrating modern machine learning frameworks with dedicated hardware accelerators, such as low-power neural processing units within wearable devices, supporting both real-time, on-device updates and periodic cloud-based retraining sessions.

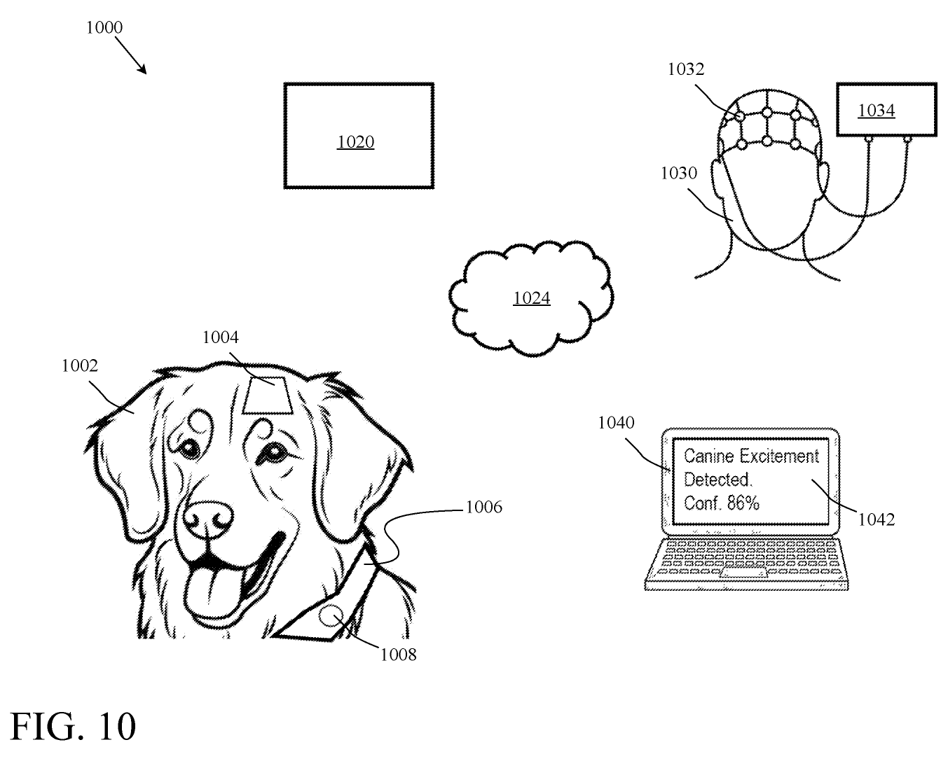

FIG. 10 shows an exemplary environment 1000 in which a system for bilateral communication between humans and non-human animals may be used, in accordance with one or more embodiments. Dog 1002 is shown, wearing a non-invasive brainwave sensor 1004. In embodiments, the non-invasive brainwave sensor 1004 may be comprised of a spray-on conductive polymer ink. In embodiments, the conductive polymer ink may include (Poly(3,4-ethylenedioxythiophene):poly(styrenesulfonate)) (PEDOT:PSS). In embodiments, the conductive polymer ink may be applied directly to the scalp or skin using a microjet printing system. In some embodiments, a small area of the dog may be shaved to expose bare skin for applying the conductive polymer ink. The ink may include additives to optimize conductivity, reduce skin impedance, and ensure mechanical durability during prolonged wear. Once applied, the ink dries into a thin, flexible film that conforms seamlessly to the skin surface, even in the presence of hair or irregular contours. In some embodiments, instead of, or in addition to, a spray-on conductive polymer ink, pre-patterned tattoo electrodes may be transferred onto the skin for neural signal acquisition. These electrodes may be composed of biocompatible materials and provide low contact impedance. In some embodiments, the epidermal tattoo sensor may include carbon nanotubes (CNTs), gold nanomaterials, and/or other suitable materials. These sensors may detect brain or muscle activity through electrical signals with high signal-to-noise ratios (SNRs), rivaling traditional invasive systems. In embodiments, the non-invasive brainwave sensor comprises a sensor comprised of conductive polymer ink. In embodiments, the conductive polymer ink comprises (Poly(3,4-ethylenedioxythiophene):poly(styrenesulfonate)) (PEDOT:PSS).

In one or more embodiments, the signals acquired by brainwave sensor 1004 are sent to a non-invasive brainwave sensor auxiliary module 1008 that may be attached to a collar 1006 worn by the dog 1002. In embodiments, the signals acquired by brainwave sensor 1004 may be sent to the brainwave sensor auxiliary module 1008 via near field communication (NFC) techniques, Bluetooth Low Energy (BLE), or other suitable techniques. One or more embodiments may utilize a custom UUID characteristic for EEG data to enable specifying the sample rate, data format, and/or other parameters for transmission of EEG data. The brainwave sensor auxiliary module 1008 may send the acquired signals to a system for bilateral communication between humans and non-human animals 1020, via network 1024. In embodiments, network 1024 may include a cellular network, WiFi network, local area network (LAN), wide area network (WAN), satellite communication network, and/or the Internet. The system for bilateral communication between humans and non-human animals 1020 may include functions and instructions to acquire brainwaves obtained by brainwave sensor 1004. The system for bilateral communication between humans and non-human animals 1020 may further include functions and instructions to perform filtering, data conditioning, and analyzing the brainwaves via machine learning models. The results of the analysis may be sent to a client device 1040 via network 1024. The client device may include a laptop computer, desktop computer, tablet computer, and/or other suitable computing device. The results produced from the system for bilateral communication between humans and non-human animals 1020 may be rendered and presented on electronic display 1042. In embodiments, the results may include an emotional state as determined by brainwave patters that are analyzed by the system for bilateral communication between humans and non-human animals 1020. In embodiments, the emotional states that are identified may include, excitement, fear, worry, anger, boredom, happiness, affection, curiosity, confidence, anticipation, submission, frustration, anxiety, loneliness, discomfort, and/or other emotional states. Embodiments may include a non-invasive brainwave sensor, wherein the non-invasive brainwave sensor is configured and disposed to obtain brainwave data from a non-human animal, and wherein the non-invasive brainwave sensor is configured to provide the brainwave data to the computing device.

The environment 1000 may further include a human 1030. The human 1030 may wear a sensor array 1032 to obtain brainwaves from the human. The brainwaves may include signals such as EEG (Electroencephalography), EMG (Electromyography), and/or specific sensory patterns for communication or training purposes. The brainwaves may include different types of brainwaves (e.g., alpha, beta, delta, and/or theta waves) that may be used to analyze cognitive states. The brainwaves may be acquired and stored by data acquisition module 1034. Data acquisition module 1034 may send the brainwave data to the system for bilateral communication between humans and non-human animals 1020 via network 1024. This may enable bilateral communication via brainwaves. As an example, the human 1030 may, via thoughts, generate brainwaves that are detected by sensor array 1032, and acquired by data acquisition module 1034. The data acquisition module 1034 may then send the acquired brainwave data to the system for bilateral communication between humans and non-human animals 1020 via network 1024, where the system for bilateral communication between humans and non-human animals 1020 analyzes the human brainwaves, and translates the brainwaves into a command or request for the dog 1002. The system for bilateral communication between humans and non-human animals 1020 may encode the command in a haptic and/or audio representation that is sent to the brainwave sensor auxiliary module 1008 via network 1024. The brainwave sensor auxiliary module 1008 may have one or more output devices that include speakers and/or haptic output devices such as vibrators and/or buzzers, to provide biofeedback stimulation to the dog. The dog may be trained to perform a command or action based on the biofeedback stimulation. As a non-limiting example, the human 1030 may think a thought about the dog 1002 sitting down. The brainwaves from the human 1030 corresponding to the thought of the dog sitting down may be detected by the sensor array 1032, and acquired by data acquisition module 1034, which relays the brainwave data to the system for bilateral communication between humans and non-human animals 1020, which may encode the command in a haptic output of three short bursts of vibration that may be felt by the dog 1002 that is wearing the brainwave sensor auxiliary module 1008 on collar 1006. The dog may be trained to sit in response to feeling the three short bursts of vibration. In this way, disclosed embodiments may provide a brain-computer interface (BCI) that may be used for human-machine interaction and/or human-animal interaction, enabling the functionality of thought-driven commands by humans for control of animals, and/or controlling machinery and/or equipment, such as prosthetics and/or virtual systems. Although a dog is shown in the exemplary environment 1000 of FIG. 10, disclosed embodiments are not limited to use with dogs. Other animals, such as cats, horses, oxen, birds, aquatic animals, and/or other capable animals may also be supported by, and/or make use of, disclosed embodiments.

The environment described in FIG. 10 represents one possible implementation, but various commercializable embodiments may be realized from the disclosed system. In some embodiments, the system may be implemented as wearable neural interface collars or headsets for pets that non-invasively read an animal’s brain signals and translate basic messages to a human-readable form. Such a device might detect patterns corresponding to hunger, emotional states, or other basic needs and then output a synthesized voice or text to the owner. The apparatus might include a lightweight sensor that a pet may comfortably wear, paired with a smartphone application for real-time translation. Similar configurations could be tailored to various companion animals, each calibrated to the species’ typical brainwave signatures.

In other embodiments, the system might be implemented as an interspecies communication earpiece or augmented reality glasses for humans. Such a device could help receive and send messages to animals. For receiving, the device might pick up processed animal signals from a cloud service or the animal’s wearable and then whisper the translation to the human or display subtitles in AR. For sending, if an animal has a known command vocabulary, the human’s speech could be converted by the system into a stimulus the animal understands. Such a real-time translation interface could aid working dog handlers, veterinary staff, or wildlife researchers.

Alternative embodiments might include robotic assistants for animal communication that serve as mediators between humans and animals. A robotic pet monitor could read an animal’s brainwaves and behavior through embedded sensors and communicate with the animal in return. If the animal is experiencing anxiety or attempting to communicate a need, the robot could notify the owner remotely. The robot might also issue commands or comforting sounds to the pet as directed by the owner or an AI system. Such implementations could be useful in scenarios like farms, zoos, or homes as advanced monitoring systems.

In further embodiments, the system may be implemented as training augmentation and behavioral insight tools. A training augmentation system might use the neural interface to gauge an animal’s cognitive or emotional state during training exercises. For example, a service dog training kit could include a non-invasive EEG harness that monitors when the dog is confused, attentive, or stressed based on brain signals. The system might provide real-time feedback to the trainer and could auto-trigger positive reinforcement the moment the dog’s brain signals indicate successful comprehension. Such closed-loop trainers might speed up learning by aligning reinforcement with the animal’s internal state rather than just external behavior.

Some embodiments may implement a multi-species communication platform using neurosymbolic AI. This could be a cloud platform or software suite that uses neurosymbolic AI to integrate data from various species and contexts, serving as a universal translator back-end that manufacturers of different devices may access. The platform might maintain a knowledge base of species-specific communication signals and use a combination of machine learning and symbolic reasoning to interpret them in context. This approach could be offered as a subscription service for products involving animal communication, with the system continuously learning as more data from different animals is processed.

In yet other embodiments, the system might be implemented as immersive AR/VR “empathy” systems that allow humans to experience a representation of an animal’s perspective. Using a combination of neural interface and AR/VR, a human could experience visual or haptic feedback corresponding to the animal’s emotional state as a form of direct communication through shared neural patterns.

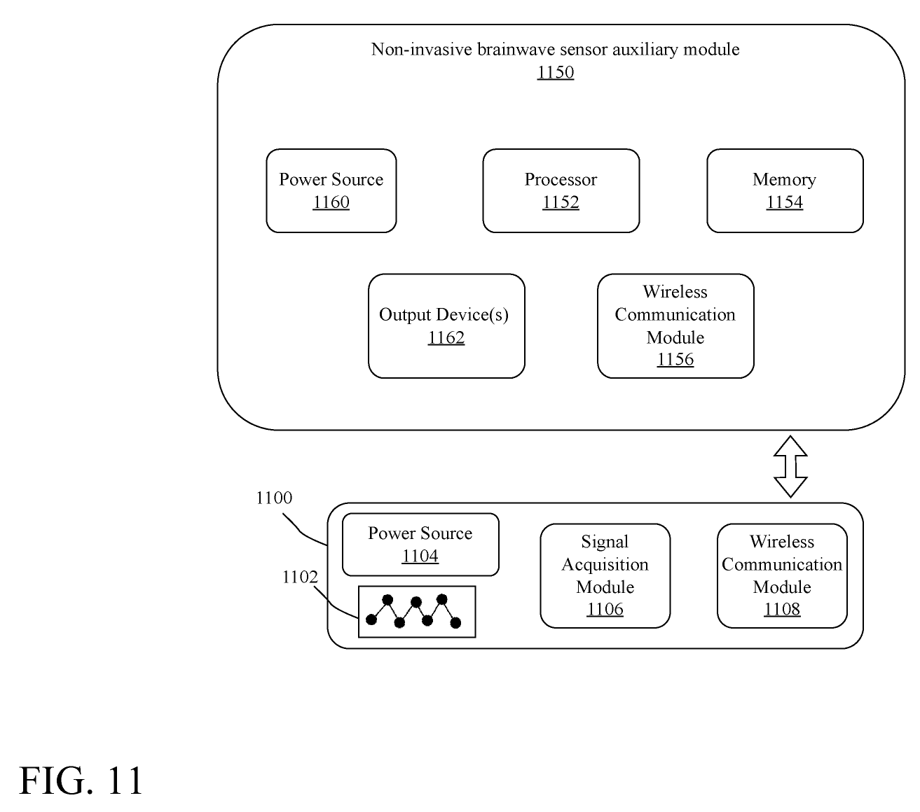

FIG. 11 shows a block diagram of an exemplary non-invasive sensor, in accordance with one or more embodiments. Sensor 1100 may include a substrate 1102 that may serve as electrodes. The substrate may include a spray-on conductive polymer ink (e.g., PEDOT:PSS) and/or ultra-flexible tattoo electrodes to map neural signals from the dog’s head. In embodiments, the substrate 1102 may be applied to the skin of an animal via a biodegradable adhesive. In some embodiments, a small area of the dog’s head may be shaved to expose a patch of skin for application of the substrate 1102. Thus, embodiments may include a flexible, biocompatible film that is applied to targeted regions of the dog’s scalp, avoiding areas of thick fur by carefully shaving an area. Other embodiments may utilize an optimized spray formula that may penetrate sparse fur layers. In some embodiments, the conductive polymer ink is doped with additives such as sodium chloride (NaCl) for low contact impedance and enhanced signal acquisition.

In embodiments, captured signals are amplified by lightweight, on-body electronics integrated into the tattoo design or attached nearby on a collar-mounted processing unit. In embodiments, the tattoo is formulated for high adhesion and stretchability to withstand the dog’s natural movements and environmental conditions, such as running, jumping, or exposure to moisture. In embodiments, the dog’s fur may be trimmed in the area where the sensor 1100 is to be applied, prior to applying the sensor 1100. Sensor 1100 may be a scalp-mounted sensor, such as depicted at 1004 in FIG. 10. In embodiments, the sensor 1100 may be applied on the dog’s scalp near the olfactory bulb. The sensor 1100 may further include a power source 1104. The power source 1104 may include a replaceable coin cell battery, rechargeable battery, and/or other suitable battery type. The battery may include a lithium-ion battery. The battery may provide power to signal acquisition module 1106 and wireless communication module 1108. The signal acquisition module 1106 may include an ADC (analog-to-digital converter) that is fed a filtered input from a filter section that may include low-pass filters to remove high-frequency noise. The signal acquisition module 1106 may further include instrumentation amplifiers, programmable gain amplifiers, and/or other suitable amplifiers for boosting weak signals for further processing. The signal acquisition module 1106 may further include a clock generator to provide timing for the ADCs. The signal acquisition module may further include a microcontroller for control of the amplifiers and/or ADCs. The microcontroller may include an ARM Cortex processor, RISC-V processor, and/or other suitable processor type.

The wireless communication module 1108 may support protocols such as Near Field Communication (NFC), Bluetooth Low Energy (BLE), RFID (Radio Frequency Identification), and/or other suitable protocols. The wireless communication module 1108 may include one or more modulators that may provide FSK (Frequency Shift Keying), ASK (Amplitude Shift Keying), and/or PSK (Phase Shift Keying) modulations. The wireless communication module may further include a microcontroller for control of the modulators and/or amplifiers and other associated components. The microcontroller may include an ARM Cortex processor, RISC-V processor, and/or other suitable processor type. In embodiments, the non-invasive brainwave sensor further comprises a wireless data transmission module. In embodiments, the wireless data transmission module includes a Bluetooth Low Energy (BLE) module.

The sensor 1100 may interoperate with the non-invasive brainwave sensor auxiliary module 1150. The non-invasive brainwave sensor auxiliary module 1150 may be a collar-mounted processing unit such as depicted at 1008 in FIG. 10. The non-invasive brainwave sensor auxiliary module 1150 may include a processor 1152. The processor 1152 may include an ARM Cortex processor, RISC-V processor, and/or other suitable processor type. The processor 1152 may be coupled to memory 1154. The memory 1154 may include a combination of random-access memory (RAM), read-only memory (ROM), Flash memory, and/or other suitable memory type. The non-invasive brainwave sensor auxiliary module 1150 may include a power source 1160. The power source 1160 may include a replaceable battery, rechargeable battery, or other suitable battery type. The non-invasive brainwave sensor auxiliary module 1150 may include a wireless communication module 1156. The wireless communication module 1156 may include components to enable communication with wireless communication module 1108 of the sensor 1100. As stated previously, this may include antennas and modulators for as Near Field Communication (NFC), Bluetooth Low Energy (BLE), RFID (Radio Frequency Identification), and/or other suitable protocols. Additionally, the wireless communication module 1156 may include components to support longer distance communication, such as WiFi, cellular network communication, and/or satellite-based communication. This may enable relay of brainwave data to the system for bilateral communication between humans and non-human animals 1020 as shown in FIG. 10. The non-invasive brainwave sensor auxiliary module 1150 may include one or more output devices 1162. The output devices may include one or more LED (light-emitting diode) lights, a speaker, a haptic device (e.g. vibrator, buzzer, etc.), and/or other suitable output devices. The LED light may convey an operational status, such as being online, offline, low battery, etc. The speaker may be used to emit sounds and/or voice data that may be heard by the dog 1002. The haptic device may impart sensations of vibration or pulsing that may be felt by the dog as stimulus in response to commands that are verbally given or otherwise conveyed by a human (e.g., human 1030 of FIG. 10). In embodiments, the dog may be trained to respond to the output from the speaker and/or haptic device to carry out commands that are provided by a human based on human language and/or thought patterns.

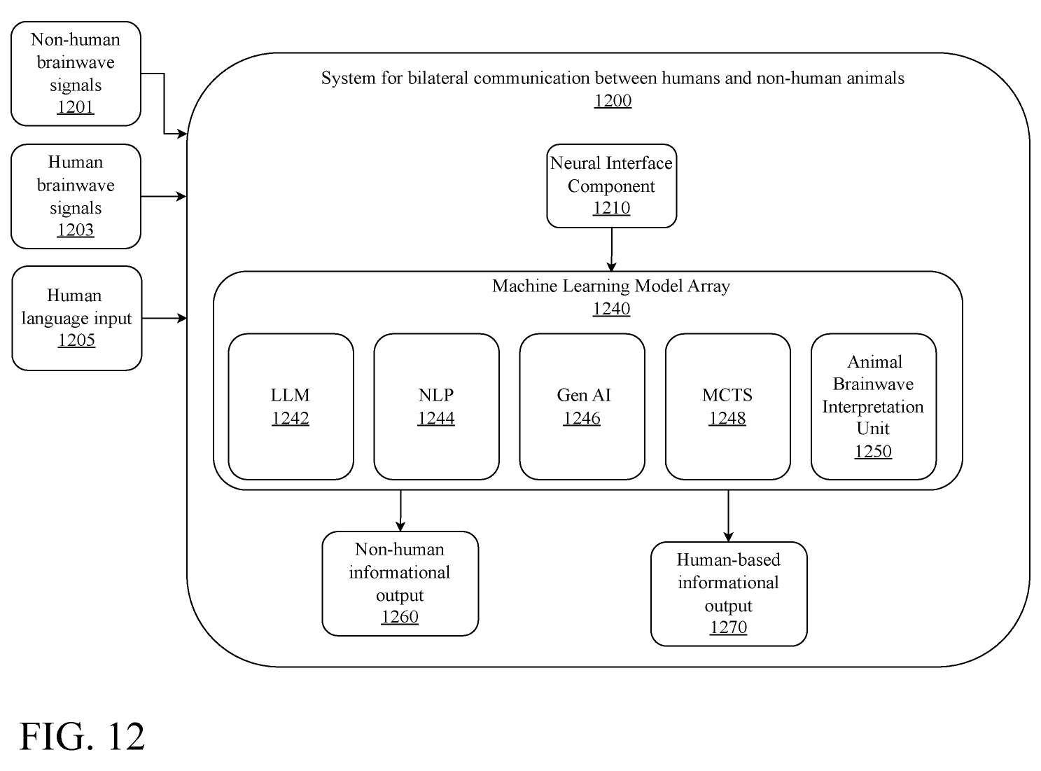

FIG. 12 is a block diagram illustrating components of a system for bilateral communication between humans and non-human animals, in accordance with one or more embodiments. System 1200 may receive as input, non-human brainwave signals 1201, human brainwave signals 1203, and/or human language input 1205. The non-human brainwave signals 1201 may include brainwave signals from animals that are obtained via non-invasive sensors such as spray-on conductive polymer inks, epidermal tattoo sensors, and so on. The animals that the brainwaves are received from may include dogs, cats, horses, oxen, primates, birds, cetaceans, and/or other suitable animals. The human brainwave signals 1203 may include brainwaves obtained from a human wearing one or more non-invasive brainwave sensors, such as depicted at 1032 in FIG. 10. The non-human brainwave signals 1201 and human brainwave signals 1203 may be input to the system for system 1200 for bilateral communication between humans and non-human animals, and the resulting output may include a non-human informational output 1260, and a human-based informational output 1270, thereby facilitating interspecies communication. The human language input 1205 may include speech and/or written communications

The system 1200 may include a neural interface component 1210. The neural interface component 1210 may enable the detection of nuanced neural responses from animals that indicate social, emotional, and environmental interactions. The animals may include land animals, such as horses, cats, and dogs. The animals may include aquatic animals, such as whales, dolphins, fish, octopus, and squid. The animals may include birds and other flying animals. In embodiments, the neural interface component 1210 may be coupled to the animals to obtain signals indicative of emotional states, and/or communication patterns. The output of the neural interface component 1210 may be input to machine learning model array 1240.

Machine learning model array 1240 may include one or more machine learning models, neural networks, and/or other systems for processing and interpreting input data. The machine learning model array 1240 may include a large language model 1242. The large language model (LLM) 1242 may be trained for specific animals (e.g., species-specific or individual-specific) and may ingest continuous streams of neural population data recorded across multiple tasks and states. These models go beyond simple language: they become multimodal encoders of animal neural signals, motor outputs, observed behaviors, and contextual cues. By structuring training data to include “high-incentive” versus “neutral” tasks, the LLM may learn when the animal’s neural signature deviates from its optimal preparatory patterns. In embodiments, the machine-learning system includes a large language model (LLM). In embodiments, the machine-learning system includes a large language model (LLM). In embodiments, the LLM includes a multi-head attention (MHA) mechanism. In embodiments, the MHA may improve self-attention by splitting the input into multiple “heads,” allowing the model to attend to different aspects of the data simultaneously. Each head processes information independently, and their outputs are combined to create a richer representation. In embodiments, the outputs from all heads may be concatenated and passed through a linear transformation to form the final representation.

The machine learning model array 1240 may include a natural language processing (NLP) module 1244. The NLP module 1244 may enable the conversion of human speech to animal-understandable patterns. The NLP module 1244 may include NLP pipelines that parse human language into semantic tokens. These tokens may then be mapped onto a species-specific “neural command embedding space.” For whales, this might involve converting a request such as ‘swim to the surface’ into a neural stimulation pattern, along with an auditory output pattern such as a song or pattern of clicks. For canines, this might involve converting a request like “Fetch the red ball” into a neural stimulation pattern plus a subtle auditory or tactile cue that aligns with the dog’s pre-trained internal representations of the action “fetch” and the visual concept “red ball.”

The machine learning model array 1240 may include a generative artificial intelligence (Gen AI) module 1246. The Gen AI module 1246 may enable supplementing training data with synthesized data, such as vocal data (e.g., canine vocalizations or whale codas), where the vocal data is created with properties such as number and regularity of signal units (clicks, barks), spectral means, and/or amplitude envelopes. The Gen AI module 1246 may include a generative adversarial network (GAN), such as WaveGAN, InfoGAN, fiwGAN, and/or other suitable GAN.

The machine learning model array 1240 may include a Monte Carlo Tree Search (MCTS) module 1248. The MCTS module 1248 may enable adaptive, look-ahead scheduling decisions. Instead of applying fixed heuristics or static load-balancing, disclosed embodiments may simulate and/or evaluate multiple future states of the pipeline before choosing the next action. By repeatedly exploring and exploiting different pipeline routing decisions (e.g., which specialist model to send partial outputs to, or how to scale certain pipeline segments), MCTS may minimize the cumulative regret over time, converging toward near-optimal scheduling policies that are robust to changing conditions, input distributions, and latency constraints. In one or more embodiments, the MCTS module 1248 may enable enhanced resource allocation, such as allocating more GPUs, selecting specialized hardware accelerators, and/or adjusting batch sizes downstream.

The machine learning model array 1240 may include an animal brainwave interpretation unit 1250. Animal brainwave interpretation unit 1250 may utilize machine learning to classify and/or interpret brainwave patterns. The animal brainwave interpretation unit 1250 may utilize and/or implement Support Vector Machines (SVMs), Principal Component Analysis (PCA), Random Forests, Convolutional Neural Networks (CNNs), Recurrent Neural Networks (RNNs), Autoencoders, and/or other machine learning components. The machine learning may be trained for tasks such as identifying abnormal brainwave patterns linked to epilepsy or cognitive decline, detecting when a dog is engaged or distracted to refine training techniques, as well as distinguishing stress, excitement, focus, relaxation, and/or other emotional states in dogs.

The machine learning model array 1240 may include, as an output, non-human informational output 1260. The non-human informational output 1260 may include audio output. The audio output may include species-specific audio waveforms such as clicks and songs for cetaceans, growling and/or barking sounds for canines, and so on. In an aquatic environment such as depicted in FIG. 1, the audio output may be provided by underwater speakers or other suitable transducers. In the terrestrial environment such as depicted in FIG. 10, audio and/or haptic output may be provided by the brainwave sensor auxiliary module 1008.

The machine learning model array 1240 may include, as an output, human-based informational output 1270. The human-based informational output 1270 may include visual information such as text and/or symbology. The human-based informational output 1270 may include audio information. The audio information may include synthesized speech, tones, and/or other sounds to convey information identified by the machine learning model array 1240. Referring again to the example depicted in FIG. 10, a human 1030 may give a thought command to the dog 1002 by thinking a thought that is detected via sensor array 1032, and provided to system for bilateral communication between humans and non-human animals 1020 as previously described. The thought command may be converted to haptic and/or audio that is output by brainwave sensor auxiliary module 1008. In this way, the non-human informational output 1260 and the human informational output 1270 may enable bilateral communication between humans and non-human animals.

In one or more embodiments, system 1200 incorporates real-time role-based task allocation, wherein task delegation is dynamically adjusted based on cognitive and emotional states derived from non-human brainwave signals 1201. The system continuously evaluates stress levels, focus states, and confidence scores for each participant and reassigns roles to ensure optimal task execution and efficiency.

For example, if system 1200 detects that a first canine exhibits high stress and reduced attention levels, the system may temporarily remove the canine from active duty and instead assign the task to a second canine exhibiting lower stress and higher engagement levels. This adaptive workload distribution ensures that each non-human participant operates within its optimal cognitive state, reducing errors and improving mission success rates.

Additionally, system 1200 facilitates collaborative knowledge sharing by enabling multiple brainwave sensor units to exchange learned insights in real-time. By leveraging distributed AI techniques, system 1200 ensures that individual learning improvements (such as improved scent detection models) are propagated across all participating agents without requiring constant human oversight.

To further optimize situational awareness, system 1200 may incorporate hierarchical decision layers, allowing localized decision-making at the individual participant level while maintaining centralized coordination for mission-critical decisions. By integrating dynamic task reallocation, cognitive-state monitoring, and distributed learning protocols, system 1200 enables an intelligent, real-time response framework for advanced human-animal collaboration scenarios.

The system for bilateral communication between humans and non-human animals 1200 may implement various signal processing techniques for effectively decoding animal neural signals. In embodiments, the system may employ Fourier Transform for frequency analysis, which may decompose neural time-series into frequency components. Animal brain signals, similar to human EEG, often contain rhythmical patterns (such as delta, theta, alpha, beta, gamma bands) that may correlate with mental states or intentions. Applying Fast Fourier Transform (FFT) or power spectral analysis may help identify these frequency-domain features. For example, a canine’s EEG might show increased beta-band activity when attentive or alpha rhythms when relaxed. Converting raw signals into a frequency spectrum allows the system to process features that may be more informative than raw waves. In some implementations, frequency analysis may serve as a baseline in classifying animal communication signals, and may be a first step to filter noise and focus on meaningful oscillations.

In various embodiments, the system may utilize Wavelet Transform for time-frequency analysis. Unlike standard FFT, wavelet transforms (such as Continuous or Discrete Wavelet Transform) may provide a time-frequency analysis, capturing how signal frequency content changes over time. Animal neural signals and vocalizations are often non-stationary, and wavelet transforms may excel at detecting transient patterns. For decoding communication, this capability may be crucial, as an animal’s “neural utterance” could comprise a sequence of temporal patterns that carry meaning. Time-frequency features may improve classification of animal signals in certain implementations. Wavelets may potentially isolate patterns or oscillatory motifs in neural data that correspond to specific behaviors or signals. In practice, the system might apply a wavelet transform on an animal’s EEG to detect when a particular frequency band rises at a specific moment, providing time-localized frequency features that enable more nuanced decoding.

The system may implement Principal Component Analysis (PCA) for dimensionality reduction in some embodiments. Neural interfaces often capture multivariate data from many electrodes and time points. PCA may reduce high-dimensional data to principal components that capture major variance. By applying PCA or related methods to animal neural signals, the system may extract dominant patterns from multiple channels. For example, if numerous EEG sensors record an animal’s brain, PCA might reveal that much of the signal variance is due to a few principal components. Using PCA features may help denoise and summarize the data for machine learning models. In certain implementations, PCA may assist in isolating neural motifs that correspond to meaningful actions or states. Before processing animal brainwave data through a deep network, the system might project it onto principal components to emphasize signal aspects that matter while reducing random fluctuations. This approach may not only improve model training by reducing overfitting on noise but also provide some interpretability, as the principal components might be visualized or understood biologically.

In certain embodiments, the machine learning model array in the system may employ advanced deep learning models, such as transformers with multi-head attention, which have demonstrated state-of-the-art performance in sequence modeling tasks and are increasingly applied to neural signals. Transformers process data through layers of self-attention, where multiple “heads” attend to different parts or aspects of the input sequence. For brainwave decoding, this means a multi-head attention network may simultaneously focus on different temporal segments, frequency bands, or sensor channels of an animal’s neural signals. For instance, one attention head might learn to attend to a pattern occurring at the beginning of a signal, while another attends to a sustained rhythm that follows. Transformer-based EEG decoders may outperform traditional CNN/RNN models in some implementations. These models may mitigate issues like noise and variable timing by learning long-range dependencies and highlighting critical features via attention. In interspecies communication, a transformer could take a sequence of neural readings from an animal and output a sequence of tokens representing the interpreted meaning. The multi-head attention would enable the model to align segments of neural activity with components of a sentence or command. Further, attention mechanisms may offer some interpretability by revealing which part of the neural signal influenced the translation.

In one or more embodiments, the machine learning model array 1240 may implement an adaptive thresholding framework for emotional state detection that dynamically adjusts sensitivity and decision thresholds based on context and feedback. This framework may ensure accuracy across individuals and environments by accounting for baseline variations and noise. The animal brainwave interpretation unit 1250 may apply adaptive thresholding to continuously track stress, pain, or well-being through the non-invasive sensors. By dynamically adjusting detection thresholds to an individual animal’s normal ranges and circadian rhythms, the system may flag abnormal emotional states in real time with high confidence, potentially enabling a diagnostic system that learns each animal’s patterns and recalibrates model parameters for each species and individual.

The adaptive thresholding framework within the machine learning model array 1240 may also drive predictive modeling of animal behavior and affective state by aggregating time-series data to anticipate actions or mood shifts. The system may employ probabilistic models with adaptive thresholds that update as confidence in a prediction changes. For instance, if particular signals typically precede an aggressive response, the system may lower the threshold for the “agitation” state when those precursors are detected. Over time, reinforcement learning techniques implemented through the Monte Carlo Tree Search (MCTS) module 1248 may optimize these thresholds based on prediction success.

For enhanced interspecies communication, the adaptive framework implemented in the natural language processing (NLP) module 1244 may enable AI translators that convey an animal’s emotional state to humans in intuitive ways. The system may learn an individual animal’s “language” by adjusting thresholds for different emotional expressions based on ongoing data. The framework may also incorporate biofeedback, using the animal’s physiological responses to human reactions to determine whether communication was effective, creating a continuous improvement loop.

In robotic planning and AI decision systems, the adaptive thresholding framework may be embedded in autonomous agents’ cognition to assess emotional cues and adjust behavior thresholds accordingly. The system may implement emotion-aware interaction protocols – guidelines or algorithms for how an autonomous agent should respond to the emotional states of nearby humans or animals. These protocols may be enabled by fast signal processing that analyzes audio, visual cues, and bio-signals in real time.

For multi-agent collaboration between humans, animals, and machines, the adaptive framework may enhance shared state estimation by including emotional state as a key component of the team’s state. The system may implement cross-modal data fusion to interpret signals of emotional and situational context and synchronize team actions accordingly. Each modality might provide independent assessments that are weighted dynamically, with contextual modality weighting applied to determine which sensors are most reliable under current conditions.

The system may further implement AI-mediated communication bridges through the human-based informational output 1270 and non-human informational output 1260 that translate and moderate signals between all parties, enhanced by emotional context. This communication bridge may not only translate signals but also inject calming or motivating stimuli to appropriate team members when needed. Throughout these applications, the adaptive thresholding framework may serve as the central nervous system for emotionally intelligent ecosystems, continuously learning and adjusting thresholds to balance sensitivity and specificity for each unique individual and situation.

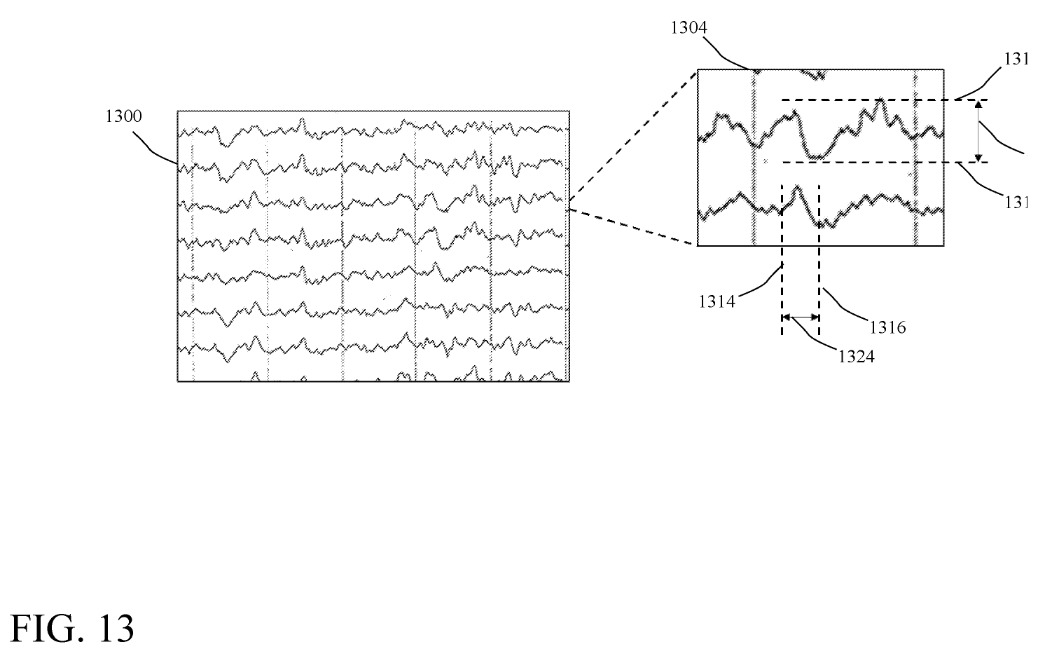

FIG. 13 shows exemplary canine brainwaves that may be analyzed using one or more embodiments. Canine brainwave signals 1300 may be obtained from a sensor such as non-invasive brainwave sensor 1004 shown in FIG. 10. The brainwaves may be representative of EEG (electroencephalography) signals. Section 1304 shows additional details of the brainwaves. The brainwave signals may have a local minimum amplitude 1312 and a local maximum amplitude 1310 with a signal differential 1322. The brainwave signals may have peaks having a starting point 1314 and an ending point 1316, defining a peak duration 1324. In embodiments, the signal differential and peak duration are used as factors in determining emotional and/or cognitive states of non-human animals.

In embodiments the canine brainwave signals 1300 may be obtained by non-invasive EEG sensors placed on the dog’s scalp that may detect electrical activity. The signals shown in FIG. 13 may be preprocessed by the system 1200 for bilateral communication between humans and non-human animals of FIG. 12. The brainwave signals 1300 may be filtered to remove noise from muscle activity, blinking, and/or environmental interference. The system 1200 for bilateral communication between humans and non-human animals of FIG. 12 may further perform feature extraction on the brainwave signals 1300, in order to extract frequency-domain and/or time-domain characteristics, including delta, theta, alpha, beta, and/or gamma waves.

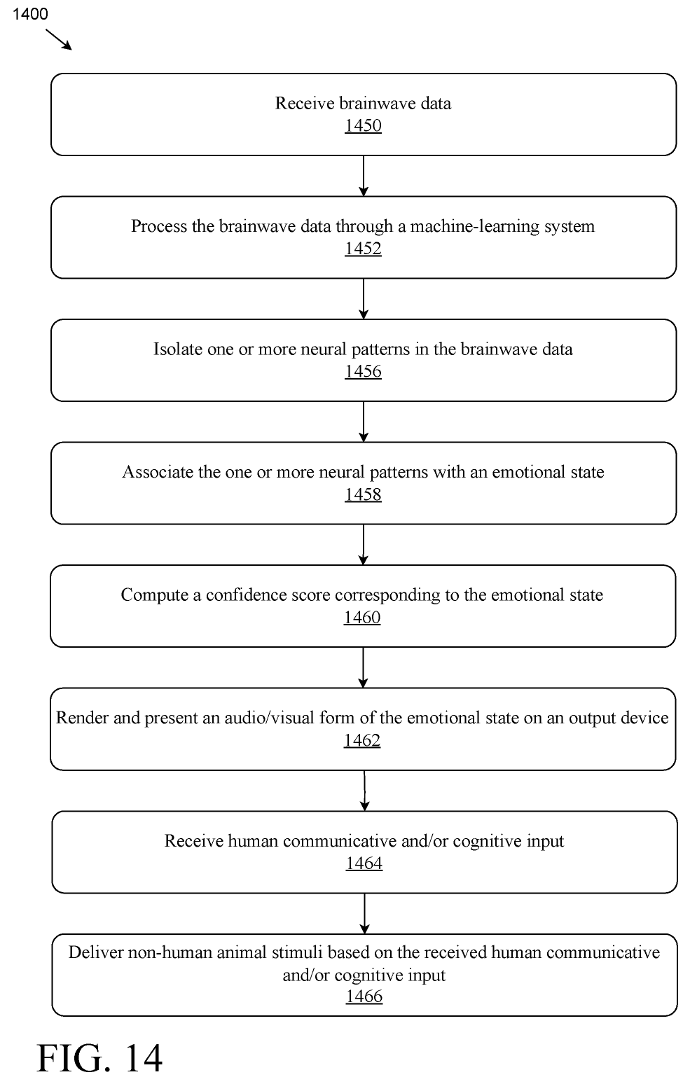

FIG. 14 is a flow diagram illustrating an exemplary method for bilateral communication between humans and non-human animals, in accordance with one or more embodiments. The method 1400 starts with receiving brainwave data at block 1450. The brainwave data may include non-human animal brainwave data. The brainwave data may include human brainwave data. The brainwave data may be acquired from one or more non-invasive sensors. The sensors may include sensors comprised of conductive polymer ink, tattoo electrodes, and/or other suitable sensors. In embodiments, the tattoos may be placed over regions of the brain associated with emotion (e.g., limbic system regions) in order to detect neural activity indicative of emotions and/or feelings such as stress, fear, and/or happiness. In embodiments, the non-invasive brainwave sensor comprises an epidermal tattoo sensor. In embodiments, the epidermal tattoo sensor comprises carbon nanotubes (CNTs). In embodiments, the epidermal tattoo sensor comprises gold nanomaterials. One or more embodiments may include high-sensitivity brainwave capture technology that may enable non-invasive capture of brainwave patterns associated with animal responses, such as attention, excitement, or stress. This setup is especially useful in applications like service dog training, where brainwave signals may indicate readiness for commands or stress levels in various environments. The method 1400 continues with processing the received brainwave data through a machine-learning system at block 1452. The machine-learning system may be trained using supervised learning techniques. Training data may include sample brainwaves obtained during known emotional states, such as fear, excitement, and/or stress. The brainwave signals may include EEG signals. The brainwave signals may be mapped to known situations, to enable creation of labeled data. The labeled data may include multiple parameters, such as observed behaviors, external stimuli, and/or physiological states. The model used may include Support Vector Machines (SVM), Random Forrest, CNNs (Convolutional Neural Networks), RNNs (Recurrent Neural Networks) / LSTMs (Long Short-Term Memory), and/or other suitable machine learning systems.

The method 1400 continues with isolating one or more neural patterns in the brainwave data at block 1456. The neural patterns may include spikes in amplitude, change in frequency, and/or other changes in characteristics of the brainwave data. The method 1400 continues with associating the one or more neural patterns with an emotional state. In one or more embodiments, transformers and attention networks may be used for associating brainwaves with emotions and feelings once the model is trained. In embodiments, the brainwave signals may be converted into structured time-series data, by segmenting the data into time windows. Raw brainwave signals may be used to build feature maps, using techniques such as Fourier Transform, Wavelet Transform, and/or other suitable techniques. Additionally, the brainwave signals may be converted into embeddings, such as multidimensional vectors. In embodiments, the embedding may be encoded as a numerical representation of brainwave signals, mapped into a multidimensional vector space. These vectors may be used to capture the temporal, spectral, and spatial characteristics of brainwave activity, making it well-suited for machine learning models to analyze patterns. The method 1400 continues to block 1458 where the one or more neural patterns are associated with an emotional state. The association may be based on the output of a machine-learning system that has been previously trained. The training may include supervised and/or unsupervised learning techniques. The method 1400 continues to block 1460 where a confidence score corresponding to the emotional state is computed. In embodiments, the computing of a confidence score includes utilizing a softmax layer within a neural network. In some embodiments, a temperature scaling function may be applied to softmax values to calibrate softmax outputs. Other embodiments may utilize Monte Carlo Dropout to determine a measure of uncertainty in the output of the machine learning system. Yet other embodiments may utilize a K-nearest neighbors (KNN) confidence estimation. Other techniques for computing a confidence score may be used in one or more embodiments. In embodiments, in response to the confidence score exceeding a predetermined threshold, the results are displayed on an output device, such as indicated at 1042 of FIG. 10. In embodiments, the confidence score is normalized to have a range of 0 – 100, with 100 indicating a high confidence and 0 indicating a low confidence.

The method 1400 continues to block 1462, where an audio/visual form of the emotional state is rendered and presented on an output device. The output device may include a computer monitor, speakers, and/or other suitable output device. Examples of rendering and presenting an audio/visual form of an emotional state is shown at 1040 of FIG. 10, where as an example, an emotional state of excitement with a confidence score of 86 percent is rendered on the electronic display 1042 of the client device 1040. The method 1400 continues to block 1464 where human communicative and/or cognitive input is received. The communicative input may include written text, uttered speech, gestures, and/or other human communication. The cognitive input may include brainwaves, such as captured from a sensor array as indicated at 1032 of FIG. 10. The method 1400 continues to block 1466 where non-human animal stimuli is delivered based on received human communicative and/or cognitive input. The non-human animal stimuli may include audio data such as tones, chirps, barking sounds, and/or other sounds. The non-human animal stimuli may include haptic data such as sensations from a buzzer or vibrator, such as may be disposed within brainwave sensor auxiliary module 1008 (FIG. 10), which may be worn on a collar 1006 (FIG. 10) to enable a dog to receive haptic and/or audio feedback based on received human communicative and/or cognitive input.

In one or more embodiments, the system refines emotional state detection by computing a normalized confidence score on a 0–100 scale, where a score of 100 represents the highest certainty that a detected neural pattern corresponds to a specific emotional state. The confidence score may be derived by integrating multiple factors, including signal quality metrics such as signal-to-noise ratio and electrode impedance, the strength of pattern matching against pre-established emotional signatures, temporal persistence and consistency of the detected signal over predefined time windows, cross-correlation with supplementary physiological indicators such as heart rate variability, and historical detection accuracy for the specific non-human animal subject. In one or more embodiments, the predetermined threshold for confirming a detected emotional state may be dynamically adjustable, with variations based on the criticality of the detected state, operational context, and the inherent signal characteristics associated with that state.

In some implementations, the system may adjust confidence thresholds based on application-specific requirements. For high-stakes scenarios, such as detecting acute distress in service animals, the confidence threshold may be set within the 90–100 range to minimize false-positive detections and ensure immediate corrective action. For routine emotional monitoring, a confidence threshold of 75 may be sufficient, while early-warning detection may employ thresholds as low as 60, flagging potential emotional states for subsequent verification. In one or more embodiments, the system may implement a multi-tiered thresholding protocol, wherein detected confidence scores trigger graduated response actions. A high confidence range (90–100) may trigger an immediate alert or action, a medium confidence range (75–89) may initiate secondary validation through additional sensor fusion or increased monitoring, a low confidence range (60–74) may result in an increased sampling rate for further data acquisition, and scores below 60 may be logged for analysis without triggering an immediate response.

In one or more embodiments, the system incorporates temporal factors into the confidence computation by requiring that the detected neural signature persist for a minimum duration before the confidence score is finalized. For example, acute states may require a 5-second persistence, while general states may require 30 seconds of continuous signal consistency. The system may apply decay factors to account for sustained signals and implement hysteresis mechanisms to prevent rapid oscillations between state classifications, thereby ensuring stability in emotional state determination. Additionally, the system may monitor the rate of change of the confidence score to differentiate between abrupt state transitions and gradual trends in neural patterns.

In some embodiments, adaptive thresholding may be achieved via a closed-loop feedback mechanism wherein the system automatically adjusts thresholds based on real-time validation against handler observations and environmental context. If repeated observations confirm that a particular emotional state, such as mild anxiety, is reliably detected at confidence scores in the 80–85 range, the system may autonomously lower the threshold for that state to 80 to enhance sensitivity. Conversely, if false positives occur, the threshold may be increased accordingly. The system may further tailor threshold adjustments based on factors such as state criticality, historical detection performance, typical signal amplitudes, and potential misclassification risks. In some embodiments, state-specific thresholds may be predefined, such as 95 for acute distress, 85 for mild anxiety, 80 for attention or focus, and 75 for general happiness, though these values may be dynamically adjusted over time.

In one or more embodiments, confidence score computation is further refined through advanced signal processing techniques. The system may apply Fourier and Wavelet transforms to extract time-frequency domain features from neural signals, while deep neural networks with multi-head attention mechanisms may dynamically weigh various input parameters. The neural networks may be trained on extensive datasets comprising annotated neural patterns and associated behavioral states, incorporating adaptive learning algorithms that allow for continuous refinement of threshold levels based on both short-term sensor data and long-term detection performance trends. In some implementations, probabilistic models and Bayesian inference techniques may be utilized to adjust confidence thresholds in real time, taking into account external environmental variables such as time of day, ambient conditions, and recent activity history of the non-human animal.

By integrating advanced thresholding methodologies with a dynamic, multi-tiered feedback loop, the system enables highly sensitive and robust emotional state detection. The use of adaptive thresholding not only optimizes detection sensitivity and minimizes false positives but also allows for a scalable and context-aware communication interface that may be tailored to species-specific requirements and varied operational scenarios. The combination of detailed signal analysis, adaptive learning techniques, and graduated response protocols represents a significant advancement over static threshold models, enabling a self-adjusting framework for bilateral communication between humans and non-human animals.

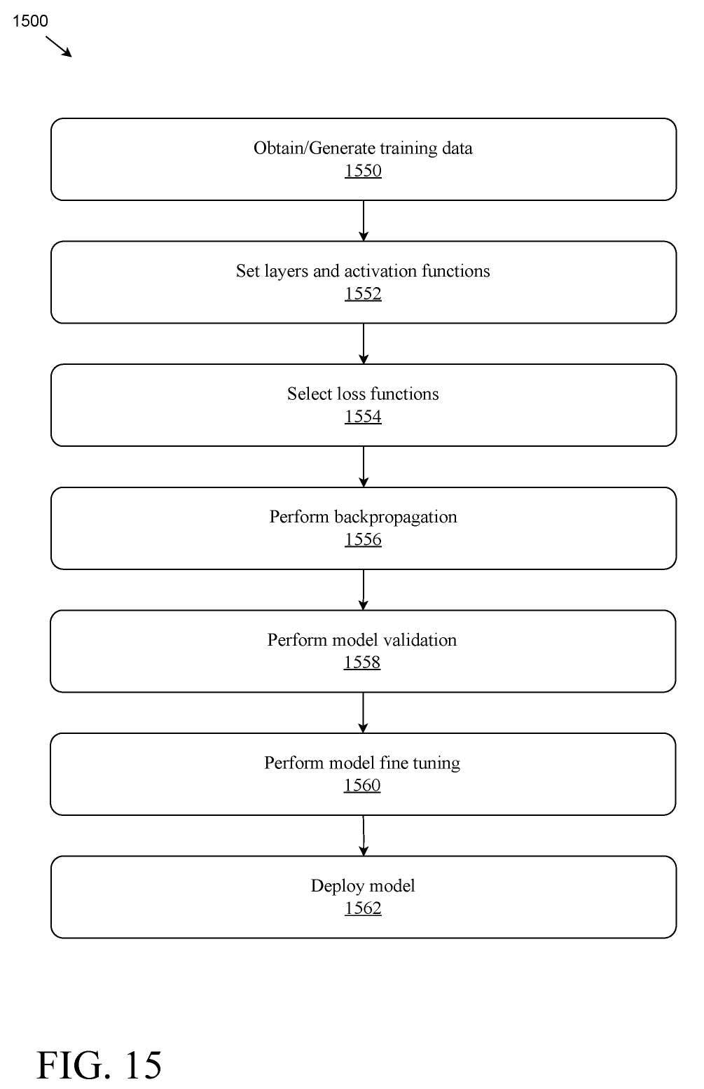

FIG. 15 is a flow diagram illustrating an exemplary method for training a system for bilateral communication between humans and non-human animals, according to one or more embodiments. The method 1500 starts with obtaining and/or generating training data at block 1550. The training data may include human communication data, non-human animal communication data, as well as synthesized data that is generated for supplementing human communication data and/or non-human animal communication data. The synthesized data may be derived from a generative AI network, such as a GAN. The method 1500 continues with setting layers and activation functions at block 1552. In a neural network, layers are the building blocks that form the structure of the network. Each layer comprises a collection of neurons (also called nodes or units), and each neuron performs a specific computation on the input data. The output of one layer becomes the input to the next layer, creating a series of transformations from the input to the output. The layers may include input layers, output layers, and/or hidden layers. The activation functions introduce non-linearity into the model, allowing it to learn and represent complex patterns in the data. In embodiments, the activation functions may include a sigmoid function, a hyperbolic tangent function, a rectified linear unit (ReLU), a Leaky ReLU, softmax function, and/or other suitable activation function. The method 1500 continues to block 1554 for selecting loss functions. The loss functions are mathematical functions used in machine learning to measure the difference between the predicted values produced by the model and the actual target values from the training data. In one or more embodiments, the loss functions may include Mean Squared Error (MSE), Mean Absolute Error (MAE), Categorical Cross-Entropy, and/or other suitable loss functions. The loss functions may be used to determine if the model is sufficiently trained. The method 1500 continues to block 1556 for training the model using backpropagation. The backpropagation process may include computing gradients of the loss with respect to the weights and biases in the output layer. These gradients are propagated backward through the neural network to the hidden layer. The method 1500 continues to block 1558, where the model is validated. The validation may include using an additional set of non-human animal communication data that was not part of the original training dataset as a test dataset. The non-human animal communication data may be translated to human communication data to confirm proper operation of the model. The method 1500 may include model fine-tuning at block 1560. The model fine-tuning may include adjusting weights and/or other hyperparameters as needed to improve model output. The method 1500 continues to block 1562, where the model is deployed for use in performing bilateral communication between humans and non-human animals.

As may now be appreciated, disclosed embodiments may provide a Brain-Computer Interface (BCI) for animals such as canines that may enable a direct communication channel between animals and humans by interpreting brain signals into meaningful data. Disclosed embodiments may enhance training, detect emotions, and enable animals to express specific needs or responses to humans without vocalizations or body language. Thus, disclosed embodiments may enable reading of brainwaves from dogs and processing them using machine learning models, potentially revolutionizing the way humans and dogs communicate. By interpreting canine brainwave (EEG) signals, disclosed embodiments may provide real-time insights into a dog’s thoughts, emotions, and intentions, creating a bridge between human and canine understanding. This may improve human-canine collaboration across multiple fields, enhancing safety, efficiency, and companionship in ways never before possible.

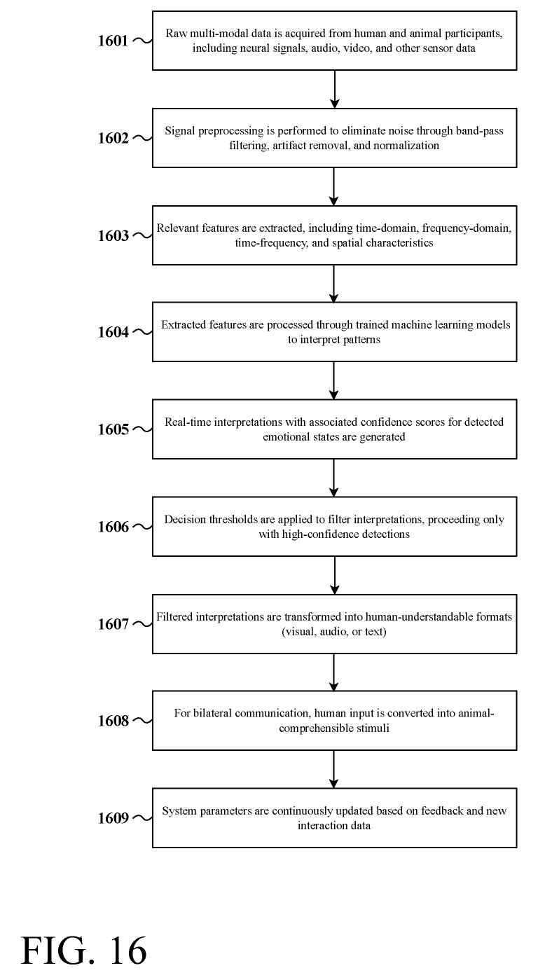

FIG. 16 is a method diagram illustrating a comprehensive process flow from data acquisition to real-time inference for bilateral human-animal communication. The system acquires raw multi-modal data from human and animal participants, collecting neural signals via neural interfaces as well as auxiliary sensor information for audio, video, or other data types, utilizing flexible conductive polymer ink or epidermal tattoo sensors applied to the animal’s skin in regions that optimize signal quality, with data acquisition components timestamping and synchronizing inputs to ensure temporal alignment of multiple data streams, potentially streaming wirelessly via Bluetooth Low Energy from the animal device to a base station 1601. The system then preprocesses these signals to clean and prepare the data for analysis, implementing procedures such as band-pass filtering to remove irrelevant frequencies, artifact removal to eliminate segments with movement artifacts or noise, normalization to adjust signal amplitude scales, and segmentation into appropriate time windows, potentially including detection of salient events such as marking points where significant changes in neural activity occur 1602. Next, the method extracts comprehensive features from the preprocessed data, deriving informative characteristics including time-domain features such as statistical measures or detection of specific neural event patterns, frequency-domain features using Fourier transforms to compute power spectral density in various frequency bands, time-frequency features applying wavelet transforms to capture transient patterns, and spatial features if multiple electrodes are used, with certain implementations employing deep feature extraction where neural networks automatically learn feature representations from raw signals 1603. The extracted features are processed through trained machine learning models that have been developed during an offline training phase where the system compiled examples linking animal neural and behavioral data to known meanings or states, with ground truth derived from labeled emotional states, controlled experiments, or unsupervised clustering, and with models potentially including deep neural networks, transformers, or hybrid systems 1604. The system generates real-time interpretations by applying the trained model to incoming features continuously, taking the current window of features and producing outputs such as emotion classifications with associated confidence scores 1605. These interpretations pass through decision thresholds or smoothing techniques to prevent spurious outputs and ensure only valid inferences proceed 1606. The system then transforms validated interpretations into human-understandable formats, presenting them as audio output such as spoken sentences, visual displays, or other appropriate modalities 1607. For bilateral implementations, the system also handles the reverse process, converting human input into forms the animal may understand, potentially through sounds or neural stimulation patterns the animal has been trained to recognize 1608. Throughout operation, the system implements supporting functions such as data storage for logging raw and processed data, error handling for managing sensor disconnections or noise issues, and multi-modal fusion for integrating different data streams, potentially requiring efficient implementation and on-device processing to maintain low latency for effective real-time operation 1609.

An advanced embodiment of the invention strategically integrates transfer learning within sophisticated machine learning architectures to facilitate swift and efficient deployment of bilateral neural-based communication systems across multiple non-human animal species. This embodiment employs a hierarchical neural network architecture explicitly designed to capitalize on transfer learning methodologies, thereby minimizing species-specific training data requirements and accelerating the development cycle.

Initially, a foundational neural network is extensively trained using comprehensive datasets from a primary animal species—for example, canine EEG data correlated to well-characterized emotional and cognitive states. This training dataset encompasses a diverse array of environmental contexts, behavioral scenarios, and emotional conditions, allowing the neural network to develop robust generalized representations of neural signal characteristics, patterns, and their associated behavioral and emotional semantic labels.

When deploying the system to additional animal species, such as equines or felines, the extensively pre-trained foundational model undergoes targeted fine-tuning utilizing considerably reduced datasets specific to the new species. This efficient fine-tuning leverages differential learning rate strategies across network layers—low-level layers, capturing fundamental EEG signal characteristics common across mammalian species, remain predominantly static, while higher-level layers, associated with nuanced interpretation and species-specific cognitive and emotional context recognition, are dynamically retrained at higher learning rates. This selective retraining ensures rapid and effective adaptation to the distinct neural patterns and contextual nuances presented by each new animal species.

Integral to the embodiment is an adaptive calibration algorithm employing active learning principles, which automatically identifies high-uncertainty neural data segments for labeling or validation, thus iteratively enhancing the classification accuracy and context-specific decoding performance of the model. This real-time adaptive mechanism optimizes both the data selection and model refinement processes, further minimizing training resources and improving the efficacy of the neural interpretation.

Additionally, this embodiment incorporates a modular software framework designed for seamless integration of specialized “language packs,” each tailored to individual species. These modules comprise customized decoding mappings (interpreting EEG-derived neural communication patterns) and encoding frameworks (delivering meaningful and species-appropriate neural or sensory stimuli). Consequently, adding support for new animal species predominantly involves software-level updates rather than substantial hardware reconfiguration or extensive retraining.

By implementing transfer learning, adaptive calibration, and modular species-specific frameworks, this sophisticated embodiment substantially enhances the scalability, flexibility, and overall practical utility of neural-based interspecies communication technologies, markedly distinguishing itself within the existing technological landscape.

An advanced embodiment of the present invention incorporates transfer learning within complex machine learning frameworks to facilitate the accelerated deployment and enhancement of bilateral neural-based communication interfaces across diverse non-human animal species. Specifically, this embodiment leverages sophisticated neural network architectures pre-trained on extensive datasets derived from neural signal recordings of a representative animal species, such as canine subjects, spanning a broad spectrum of emotional states, cognitive engagements, and environmental contexts. These comprehensive datasets enable the initial neural network architecture to establish robust, generalized representations of neural signatures and their associated semantic interpretations.

When adapting the system for additional animal species, including but not limited to equines or felines, the invention implements targeted retraining strategies. Specifically, this involves selectively fine-tuning upper layers of the neural network architecture at higher learning rates, thus efficiently adapting the neural decoding mechanisms to accommodate species-specific neural pattern variations and cognitive contextual subtleties. Lower layers, preserving foundational features learned from extensive initial training, remain largely fixed, leveraging cross-species similarities in neural signal representation.

A salient aspect of this invention is its utilization of personalized adaptive calibration and continuous learning methodologies. Upon initial integration with an individual animal subject, a structured calibration protocol is employed wherein the animal experiences controlled, known stimuli (such as interactions involving play, provision of food, or exposure to mild stressors) while neural data is concurrently captured by the device. This rigorous calibration procedure establishes a personalized baseline neural dataset reflective of distinct neural correlates corresponding to specific emotional and cognitive states and associated behavioral responses particular to the individual animal.

Following calibration, the system applies an adaptive confidence-scoring algorithm during active deployment, continuously evaluating the certainty of neural signal interpretations in real time. Interpretations exceeding a predefined confidence threshold inform actionable system outputs, while lower-confidence interpretations are flagged for further refinement or ignored to prevent erroneous outputs. Additionally, the invention integrates an active learning framework through which neural decoding models progressively refine their interpretive precision by incorporating newly acquired operational data selectively validated through the confidence-scoring process. These refinements are carefully controlled, ensuring only high-confidence data is incorporated, thereby preserving model accuracy and minimizing the risk of degradation from unreliable or ambiguous inputs.

The modular architecture further facilitates scalability via specialized software modules, termed “language packs,” designed for efficient deployment across various species. These modules encapsulate tailored decoding schemes and species-specific sensory stimulus mappings, thus enabling rapid and flexible adaptation to new species without extensive retraining or hardware modifications.

Collectively, these sophisticated adaptive mechanisms and modular frameworks confer the invention with remarkable scalability, individualized precision, and long-term operational robustness, representing a significant advancement in the domain of interspecies neural communication technologies. The continuous, personalized learning capability uniquely positions this invention as a dynamic, context-aware, and self-evolving communication interface, precisely attuned to the evolving neural profiles of individual animals.

In another exemplary embodiment of the invention, a robust and intelligent multi-modal fusion engine is presented, characterized by adaptive, context-sensitive interpretation of diverse sensor data streams to ensure highly accurate real-time communication between humans and non-human animals. This embodiment utilizes advanced Bayesian or transformer-based algorithms, dynamically integrating neural EEG signals, visual imagery, auditory inputs, inertial measurement unit (accelerometer) data, thermal imaging, and environmental sensors, thus offering a comprehensive assessment of the animal’s emotional, cognitive, and behavioral states.

At the core of this embodiment lies a sophisticated Bayesian inference framework that continuously updates its probabilistic reasoning based on evolving context. The system constructs an elaborate hierarchical probabilistic graphical model—a Bayesian network—where each distinct sensor modality provides evidence toward the network’s probabilistic hypotheses regarding the animal’s internal states. This hierarchical model allows for the probabilistic integration of heterogeneous data streams, dynamically assigning relevance weights to each modality based on real-time contextual cues.

For instance, upon the detection of heightened arousal in neural EEG readings, the Bayesian inference module evaluates complementary sensor inputs—such as accelerometer data for movement intensity, video analytics assessing body language, audio analysis identifying vocalizations, or thermal imaging indicating physiological changes—to accurately differentiate between genuine emotional excitement and physiological responses related to physical exertion. In cases where the neural EEG indicates excitement but accelerometer data simultaneously records vigorous physical activity, the model adaptively downweights the EEG arousal indicators to avoid misclassification, instead relying more heavily on complementary sensor streams.

Alternatively, the invention may leverage transformer-based neural architectures incorporating sophisticated attention mechanisms to achieve real-time adaptive fusion of sensor modalities. Each sensor data stream is first independently processed to extract deep feature embeddings, capturing distinct spatiotemporal characteristics inherent in neural, visual, auditory, and physiological sensor data. These feature embeddings are then integrated via contextually responsive attention mechanisms, which dynamically modulate inter-modal relevance weights in real-time based on temporal and contextual relationships detected within incoming data streams.

In such transformer-based embodiments, attention mechanisms are trained to identify correlations and predictive cues across diverse sensory inputs—such as video-based behavioral analysis, acoustic signals indicating vocalizations or environmental sounds, and physiological data including heart rate or thermal imaging signals. During operational scenarios, for instance, in periods of minimal physical activity detected by video and motion sensors, attention layers dynamically elevate reliance on EEG and subtle physiological signals, ensuring accurate interpretations of the animal’s mental and emotional states without interference from motion artifacts or environmental distractions.

Further enhancing this embodiment, an integrated adaptive context module employs advanced machine learning techniques, including temporal Bayesian updating and contextual adaptive priors, to continuously monitor and update contextual information—such as time of day, historical behavioral patterns, and environmental factors. For instance, if historical data indicate that the animal consistently experiences heightened excitement during specific routine events (e.g., feeding times or familiar social interactions), the system automatically adjusts its probabilistic priors and predictive weighting strategies to reflect these known patterns, thereby producing interpretations aligned with nuanced, individualized contexts rather than static sensor thresholds.

Moreover, the system includes safeguards within the adaptive updating mechanism to mitigate risks associated with overfitting or contextual drift. These safeguards include mechanisms such as regularized updates, uncertainty-based sample weighting, and periodic recalibration with baseline contextual parameters, ensuring that adaptations improve interpretive accuracy without introducing systematic biases or inaccuracies.

Through the integration of these advanced context-aware, multi-modal fusion strategies—including Bayesian hierarchical modeling, transformer-based attention mechanisms, and adaptive contextual learning—the invention significantly enhances interpretive accuracy, robustness, and reliability beyond that achievable by simpler threshold-based systems. Consequently, this embodiment establishes an innovative benchmark in the domain of interspecies communication technologies, offering unprecedented levels of interpretative fidelity, adaptability, and real-time responsiveness tailored specifically to the complex demands inherent in interspecies neural communication applications.

In an advanced embodiment of the present invention, a user-centric augmented reality (AR) visualization platform is introduced, designed to significantly enhance the intuitive accessibility and interpretability of animal-derived neural communication data for diverse human users, ranging from veterinarians and researchers to pet owners and handlers. This embodiment incorporates an augmented reality dashboard compatible with commonly available consumer and professional devices, including smartphones, tablets, and specialized AR glasses or headsets, effectively democratizing sophisticated interspecies neural communication technologies.

The AR visualization interface integrates real-time neural decoding outputs, sensor-derived multi-modal interpretations, and contextual analyses to represent the animal’s emotional, cognitive, and behavioral states via intuitive and immediately comprehensible visualizations. Central to this embodiment is the real-time rendering engine that synthesizes neural, behavioral, and environmental data into simplified, visual, and symbolic representations specifically optimized for rapid cognitive assimilation by human users. For example, emotional states detected by EEG patterns are depicted as intuitive visual icons or symbolic emoticons (such as smiling, neutral, or distressed faces), displayed directly within the user’s augmented field of view.

When the neural-based system detects significant emotional, cognitive, or physiological states—such as anxiety, pain, stress, or alertness—the AR visualization system instantaneously projects clear, actionable alerts and recommendations directly onto the user’s visual interface. For instance, upon identifying signals consistent with animal distress through EEG and multimodal sensor fusion analysis, the system overlays a visual notification accompanied by a numeric confidence indicator—e.g., “Anxiety Detected (92 % confidence).” Further, the AR visualization includes context-specific actionable suggestions such as “Check paws for injury” or “Evaluate environmental stressors,” thus transitioning from merely diagnostic data presentation toward proactive and pragmatic assistance.

Moreover, this embodiment leverages real-time contextual situational awareness derived from integrated multi-modal sensor inputs—including location-based data, temporal routines, environmental parameters, and historical behavioral trends—to dynamically adjust the visualization presented to the human operator. The AR interface may proactively highlight critical or urgent animal states, emphasizing visually conspicuous alerts or dynamic graphical elements to immediately capture the user’s attention and prompt timely intervention.

Additionally, advanced features such as auditory alerts, haptic feedback, and voice interaction are incorporated into this augmented reality interface, further enhancing the user’s situational awareness and responsiveness. These multimodal outputs are contextually synchronized with visual AR overlays, offering users versatile channels for receiving essential notifications in diverse operational scenarios, such as noisy environments or when visual attention is momentarily diverted.

Moreover, the AR interface employs machine learning-driven context adaptation, continuously learning user preferences, response patterns, and task-specific requirements to personalize the displayed content and interaction modalities. Such adaptive personalization ensures the continuous refinement of user experience, optimally aligning the interface design with specific professional requirements or personal preferences over extended usage periods.

Collectively, this embodiment provides a robust, sophisticated, yet highly intuitive AR-driven user interface designed explicitly to enhance real-world adoption and utilization of neural communication technologies, transforming raw sensor data into immediately actionable insights, thus significantly improving the efficacy and efficiency of human-animal interactions across numerous practical applications.

In an additional exemplary embodiment, the invention discloses an advanced integration framework serving as an extensive Internet of Things (IoT) hub, purposefully designed to interconnect seamlessly with pre-existing animal management systems and environmental control infrastructures. This embodiment transitions the neural-based animal communication apparatus from a discrete wearable technology into a central integration node within comprehensive IoT ecosystems, extensively utilized by pet caretakers, livestock administrators, conservation specialists, and veterinary experts.

Fundamental to this embodiment is a modular middleware platform architected explicitly to standardize data transmission protocols and interoperability among a myriad of third-party animal monitoring and management systems. By leveraging established IoT communication standards—including but not limited to MQTT, Zigbee, Z-Wave, Bluetooth Low Energy (BLE), and Wi-Fi—the neural communication interface facilitates seamless and secure bidirectional data exchanges with various external systems, including livestock management software, GPS-enabled animal tracking collars, and wearable health monitoring devices. Such integration markedly enriches the data streams available for advanced interpretation and provides actionable insights for improved animal care.

In practical pet management scenarios, the system operates by detecting heightened stress or anxiety states in an animal through sophisticated analysis of EEG neural patterns and associated physiological indicators. Upon recognition of these specific neural signatures, the system autonomously initiates context-sensitive responses via cloud-connected smart home services. For instance, it activates calming ambient auditory stimuli through integrated smart audio systems or modulates environmental lighting configurations utilizing interconnected smart lighting devices to foster a tranquil setting. Concurrently, the system conveys immediate alerts to pet owners through personalized smartphone applications, offering precise situational awareness and enabling swift, informed interventions remotely.

In agricultural settings, this IoT hub supports advanced operational decision-making by integrating real-time neural interface data with farm management software systems. If indicators of stress, health abnormalities, or behavioral anomalies are detected within livestock populations—ascertained through combined neural and physiological sensor analyses—the system automatically transmits relevant data into existing farm management dashboards. Subsequently, pre-programmed automated responses are triggered, including environmental adjustments, modification of nutritional schedules, or issuance of timely alerts to farm personnel, facilitating rapid evaluation and intervention to optimize animal health, welfare, and overall herd productivity.

The technological architecture integrates secure cloud-based middleware frameworks, utilizing robust IoT communication protocols such as MQTT and RESTful APIs, ensuring seamless and secure interoperability across diverse external devices and platforms. Rigorous cybersecurity provisions are incorporated, including advanced encryption methodologies and secure authentication protocols, to protect the privacy and integrity of animal-derived data throughout real-time data transmission and persistent storage.

Additionally, the embodiment incorporates sophisticated machine learning-driven contextual analytics, capable of intelligent predictive analytics and automated triggering of IoT ecosystem responses based on simultaneous evaluation of neural and environmental sensor inputs. Thus, the system not only amalgamates multiple data streams but also dynamically orchestrates intelligent, context-aware automated actions across interconnected smart devices and systems.

Through strategic positioning as an integrative IoT hub, the neural-based animal communication system substantially expands its operational utility, scalability, and applicability across numerous domains, providing a robust, intelligent integration nexus that markedly complements and enhances current animal welfare, management, and caregiving technologies.

The predictive module explicitly integrates historical and real-time multimodal data—including neural signals (e.g., EEG from flexible graphene e-tattoos), physiological signals (heart rate, respiratory rate, galvanic skin response), and environmental sensor inputs (temperature, humidity, ambient sound, visual analytics, atmospheric pressure, and air quality measurements)—to proactively anticipate animal emotional and behavioral states. This predictive engine utilizes advanced machine learning architectures such as recurrent neural networks (RNNs), convolutional neural networks (CNNs), transformer models, and hybrid neurosymbolic architectures trained on comprehensive datasets. These datasets correlate multimodal sensor inputs with annotated and empirically observed animal emotional states, providing a robust foundation for accurate forecasting of animal responses to varying environmental and internal stimuli.

In operation, the predictive module continuously processes incoming multimodal data streams, integrating them in real-time through sophisticated machine learning algorithms. Recurrent neural networks (RNNs) including long short-term memory (LSTM) or gated recurrent units (GRU) analyze these streams, identifying subtle temporal sequences and correlations predictive of future states such as anxiety, stress, excitement, or aggression. For instance, historical data associating neural signatures and environmental triggers—like thunderstorms, fireworks, or sudden loud noises—may be analyzed by the predictive algorithm to reliably anticipate anxiety episodes before overt behavioral manifestations become apparent. Additionally, convolutional neural networks (CNNs) and transformers further enhance the system’s predictive capabilities by effectively capturing spatial-temporal patterns and nuanced context within the complex interplay of sensor inputs.

Upon detecting high-confidence predictive signals indicative of potential emotional or behavioral disturbances, the predictive module initiates tailored and preemptive interventions designed to mitigate or even prevent the anticipated state. Such interventions are carefully calibrated according to the species-specific and individual animal’s known preferences, historical responses, and sensitivity thresholds. Examples include gentle haptic feedback through wearable devices to provide soothing tactile stimulation, calming auditory cues emitted through localized speakers or bone-conduction transducers, and controlled sensory inputs—such as adjustable lighting, temperature modulation, or environmental masking sounds—to immediately influence the animal’s sensory perception, thus effectively preempting escalation of negative emotional states.

Furthermore, the predictive module incorporates advanced adaptive machine learning mechanisms that ensure continuous improvement of predictive accuracy and reliability. These mechanisms systematically compare predicted states against actual observed emotional or behavioral outcomes, refining the system through incremental learning algorithms. Techniques such as semi-supervised learning, reinforcement learning, and neurosymbolic reasoning frameworks facilitate real-time updating of predictive models based on the ongoing accumulation of data. Discrepancies between predicted and actual states dynamically trigger recalibration, enabling the predictive models to progressively adapt to individual animals’ evolving neural, physiological, and behavioral profiles, as well as changing environmental conditions throughout their lifetimes.

Scalability and versatility of the predictive module are notably enhanced through sophisticated use of transfer learning methodologies and universal semantic embedding architectures. These machine learning techniques allow efficient adaptation and cross-species predictive capability by identifying and exploiting common emotional or behavioral signatures shared across multiple species. Universal semantic embeddings enable rapid calibration for new animal species, minimizing the requirement for extensive retraining by leveraging existing cross-species neural and behavioral patterns. Consequently, the predictive module may swiftly accommodate new species or individual animals by recalibrating existing models with minimal supplementary training data, significantly reducing deployment time and cost.

Additionally, to enhance contextual intelligence, the predictive module may utilize neurosymbolic reasoning frameworks and semantic knowledge bases, which explicitly encode domain-specific knowledge (e.g., typical stress triggers, common behavior cues, species-specific responses). Such frameworks allow the predictive algorithms to reason logically about sensor data, environmental contexts, and behavioral history, providing deeper understanding and more nuanced predictive capabilities than purely data-driven methods alone. Incorporating semantic and symbolic reasoning enables real-time interpretation of ambiguous or conflicting multimodal data inputs, significantly reducing false-positive predictions and ensuring actionable predictive insights tailored precisely to the animal’s current situational context.

Overall, by robustly integrating advanced machine learning techniques, adaptive continuous learning, and sophisticated environmental-contextual predictive models, this predictive module significantly advances real-time animal welfare management, improves operational readiness in working animal applications, and enhances effective cross-species communication systems. This combination ensures the invention remains a pioneering and versatile solution suitable for diverse applications such as veterinary care, wildlife conservation, animal-assisted therapeutic interventions, and operational management of working animals across various challenging scenarios and environments.

Cross-Species Semantic Embeddings represent a fundamentally novel component of the invention by providing a computational framework that translates neural patterns from diverse animal species into a unified semantic representation space. This innovative approach relies on advanced embedding techniques, including deep metric learning, contrastive learning, and transformer-based semantic encoders, to map neural data—such as EEG patterns captured through flexible graphene-based e-tattoo sensors—into high-dimensional embeddings. These embeddings encode complex emotional, cognitive, and intentional states that transcend species-specific neural architectures, effectively enabling a universal ‘language’ that bridges communication between humans and animals, or even among animals of different species.

The semantic embedding model is initially trained using extensive multimodal neural and contextual data collected across various species under controlled conditions. This initial training leverages transformer architectures to capture intricate temporal and contextual relationships among neural patterns, physiological data, and observed behaviors or environmental cues. By embedding data points corresponding to similar emotional or cognitive states from multiple species closely together within this shared semantic space, the model inherently learns cross-species generalizable features—such as neural signatures indicative of anxiety, curiosity, excitement, or attention. The embeddings therefore represent fundamental psychological states independent of species-specific neural configurations, effectively creating a universal neural “language” that bridges the gap between distinct neural anatomies and sensory experiences across species.

After initial training, the semantic embedding model provides a versatile foundation for rapidly integrating new species or individuals into the communication framework. When onboarding a new species, the invention employs a minimal calibration dataset comprising representative neural and environmental contexts. Utilizing transfer learning techniques, the pre-established semantic embedding space efficiently accommodates the novel neural signatures without extensive retraining. The cross-species embeddings inherently encode shared conceptual meaning rather than species-specific neural characteristics, thus significantly reducing the required training complexity and enabling rapid deployment and effective adaptation of the invention to numerous animal species, whether domestic, working, or wild.

Furthermore, these cross-species semantic embeddings inherently enhance the system’s interpretability and transparency. Each embedding dimension is associated with high-level semantic concepts, such as anxiety, curiosity, alertness, or fatigue, that are recognizable across species boundaries. This interpretability allows handlers, veterinarians, and conservationists to intuitively understand neural signals without requiring extensive technical expertise in neural decoding. Additionally, embedding interpretability supports ethical use by facilitating transparency in decision-making processes, ensuring that interventions based on predicted emotional states are easily justified and monitored.

Finally, continuous refinement of the semantic embeddings is integrated into the invention through online adaptive learning processes. As real-time data streams from field deployments become available, the embedding space dynamically adjusts using online learning methodologies—such as neurosymbolic reinforcement learning—to account for evolving neural patterns and environmental interactions. Over time, this approach systematically enriches the embedding space with increasingly diverse interspecies data, progressively enhancing predictive accuracy and reducing the required calibration for future expansions. The cross-species semantic embeddings thus provide a self-evolving communication paradigm, setting a groundbreaking standard for interspecies neural communication that bridges neurological, ecological, and technological disciplines in unprecedented ways.

The embedding model also incorporates a dynamic, real-time adaptive learning mechanism that continually updates its representations through reinforcement and neurosymbolic learning approaches. Continuous real-time feedback derived from comparing predictive outcomes against observed animal responses iteratively refines the embedding space, enhancing the precision and predictive capability of the system. Consequently, the semantic embeddings adapt to individual and species-specific neural dynamics, environmental changes, and novel behaviors as they emerge, ensuring sustained accuracy and relevance throughout the operational lifespan of the invention.

Furthermore, the invention explores various data representation strategies—such as token-based, byte-level, or hybrid intermediate tokenization—to optimize semantic embeddings across heterogeneous data types. Token-based methods enable explicit mapping of discrete cognitive states or neural signatures into semantic tokens, enhancing the clarity of interpretation. Byte-level representations afford maximum granularity in capturing nuanced, continuous neural variations, particularly beneficial for detecting subtle cognitive or emotional fluctuations. Intermediate tokenization combines both approaches, employing token-based abstractions for broader semantic categories, while finer-grained, byte-level data precisely encode detailed neural and physiological nuances. This hybrid approach balances interpretability and precision, offering the greatest flexibility in accommodating diverse neural architectures and multimodal data streams across varied animal species.

Overall, cross-species semantic embeddings establish an unprecedented neural communication paradigm, fundamentally transforming human-animal interaction through robust, scalable, and interpretable computational methods that span neurological, ecological, and technological boundaries.

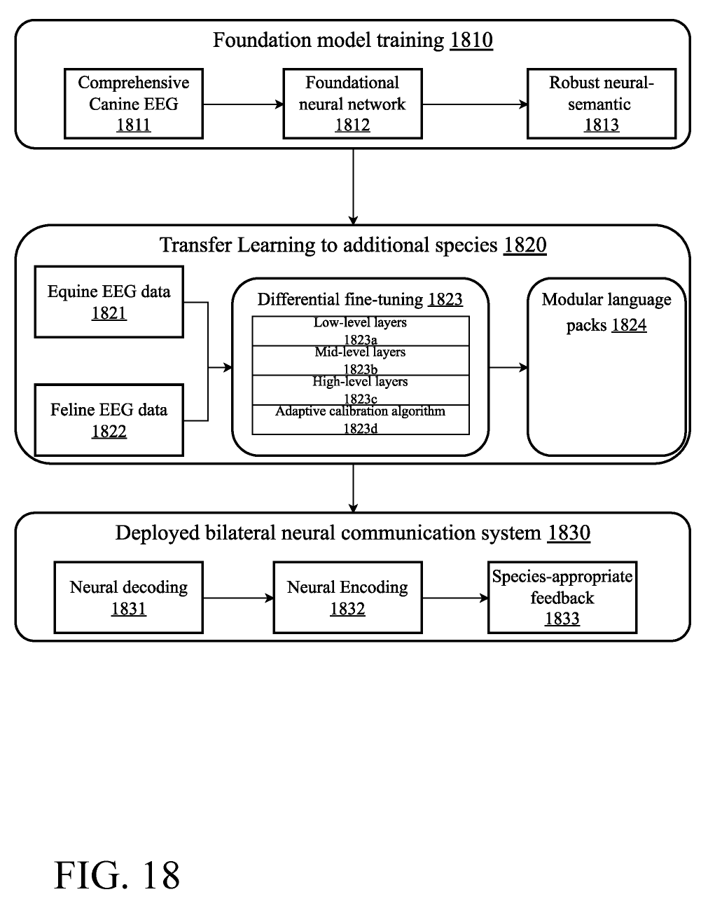

FIG. 18 is a block diagram illustrating a transfer learning for neural-based interspecies communication system, in accordance with one or more embodiments. The diagram methodically delineates the system’s architecture across three hierarchically organized phases, each with distinct components and interconnections that collectively enable efficient cross-species neural communication.

The foundational training model process utilizes canines as the primary species. The foundational stage begins with a comprehensive collection of canine EEG datasets 1811 which includes multiple environmental contexts, diverse behavioral patterns, and a wide spectrum of emotional states. These rich datasets feed into the foundational neural network 1812, which contains distinct processing layers—low-level layers and mid-level layers—that progressively extract and refine neural signal features. The output of the extensive training process is the robust neural-semantic mappings 1813 which establish generalized representations of neural signal characteristics, detailed signal-behavior correlations, and sophisticated emotional state recognition patterns.

Within the transfer learning methodology 1820, the reduced specifies-specific datasets for both equines and felines 1821/1822. These limited datasets connect to the central differential fine-tuning 1823 which presents a detailed stratification of neural network architectures across distinct layers 1823a-d. The low-level layers 1823a remain predominantly unchanged as they capture “fundamental EEG signal characteristics common across mammalian species;” mid-level layers 1823b that are extensively retrained to capture the “distinct neural patterns and contextual nuances” specific to each species; the high-level layers 1823c that are extensively retrained to capture the distinct neural patterns and contextual nuances specific to each species. The adaptive calibration algorithm 1823d (active learning) represent a system that automatically identifies high-uncertainty neural data segments for labeling or validation. The modular language packs 1824 allows for architecture with individual modules for canine, equine, and feline species, directly corresponding to the “modular software framework” and “customized decoding mappings.”

The deployed bilateral neural communication system 1830 is divided into three interconnected functional components: neural decoding 1831 (transforming EEG-derived neural patterns to semantic meaning), neural encoding 1832 (converting intended meaning into appropriate stimuli), and species-appropriate feedback 1833 mechanisms. These directly reflect the “decoding mappings” and “encoding frameworks” that deliver meaningful and species-appropriate neural or sensory stimuli.

An advanced embodiment of the invention strategically integrates transfer learning within sophisticated machine learning architectures to facilitate swift and efficient deployment of bilateral neural-based communication systems across multiple non-human animal species. This embodiment employs a hierarchical neural network architecture explicitly designed to capitalize on transfer learning methodologies, thereby minimizing species-specific training data requirements and accelerating the development cycle.

Initially, a foundational neural network is extensively trained using comprehensive datasets from a primary animal species—for example, canine EEG data correlated to well-characterized emotional and cognitive states. This training dataset encompasses a diverse array of environmental contexts, behavioral scenarios, and emotional conditions, allowing the neural network to develop robust generalized representations of neural signal characteristics, patterns, and their associated behavioral and emotional semantic labels.

When deploying the system to additional animal species, such as equines or felines, the extensively pre-trained foundational model undergoes targeted fine-tuning utilizing considerably reduced datasets specific to the new species. This efficient fine-tuning leverages differential learning rate strategies across network layers—low-level layers, capturing fundamental EEG signal characteristics common across mammalian species, remain predominantly static, while higher-level layers, associated with nuanced interpretation and species-specific cognitive and emotional context recognition, are dynamically retrained at higher learning rates. This selective retraining ensures rapid and effective adaptation to the distinct neural patterns and contextual nuances presented by each new animal species.

Integral to the embodiment is an adaptive calibration algorithm employing active learning principles, which automatically identifies high-uncertainty neural data segments for labeling or validation, thus iteratively enhancing the classification accuracy and context-specific decoding performance of the model. This real-time adaptive mechanism optimizes both the data selection and model refinement processes, further minimizing training resources and improving the efficacy of the neural interpretation.

Additionally, this embodiment incorporates a modular software framework designed for seamless integration of specialized “language packs,” each tailored to individual species. These modules comprise customized decoding mappings (interpreting EEG-derived neural communication patterns) and encoding frameworks (delivering meaningful and species-appropriate neural or sensory stimuli). Consequently, adding support for new animal species predominantly involves software-level updates rather than substantial hardware reconfiguration or extensive retraining.

By implementing transfer learning, adaptive calibration, and modular species-specific frameworks, this sophisticated embodiment substantially enhances the scalability, flexibility, and overall practical utility of neural-based interspecies communication technologies, markedly distinguishing itself within the existing technological landscape.

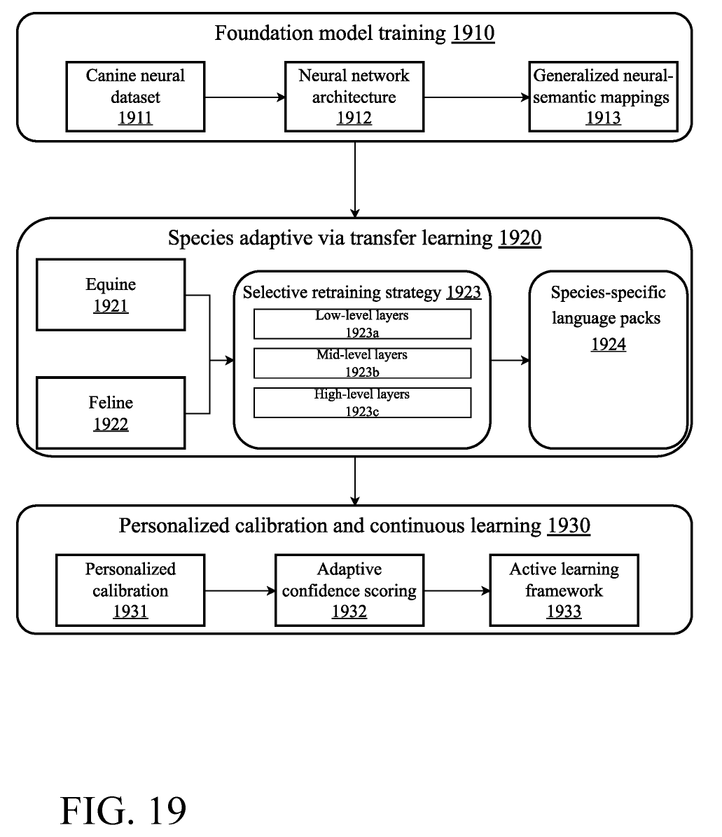

FIG. 19 is a block diagram illustrating a bilateral neural-based interspecies communication system with transfer learning. The foundational model training 1910 process forms the backbone of the system. This begins with the canine neural dataset 1911, which illustrates the comprehensive EEG recordings spanning diverse emotional states, multiple cognitive tasks, and various environmental contexts. These extensive datasets feed into the centrally positioned neural network architecture 1912, which within the architecture, the upper layers are designed to capture species-specific features, middle layers which establish fundamental neural signal processing capabilities. The output of this extensive training process manifests as generalized neural-semantic mappings 1913, which encompass robust neural signal representations, emotional state correlates, and behavior-signal associations that serve as the foundation for cross-signal adaptation.

Within the species adaptation via transfer learning methodology 1920 the limited datasets from additional species (equine and feline) 1921/1922, emphasizing the efficiency of the transfer learning approach that requires significantly less training data for new species. These connect to the central selective retraining strategy 1923, which precisely delineates the differential learning rate approach across three neural network layers: the upper layers 1923c undergo high learning rate adaptation to accommodate species-specific neural patterns; 1923b middle layers receive moderate tuning; while lower foundational layers 1923a remain fixed to leverage cross-specific neural signal similarities The modular species-specific language packs 1924 architecture with individual decoding modules for each animal species (canine, equine, and others), directly corresponding to the specialized software modules that facilitate efficient cross-species deployment without extensive hardware modifications.

Within the personalized calibration and continuous learning processes 1930, the personalized calibration 1931 protocol is where controlled stimuli (play interactions, food provision, and mild stressors) establish an individual baseline neural dataset for each specific animal subject. The adaptive confidence scoring 1932 visualizes the real-time neural signal interpretation evaluation system, with confidence levels: high-confidence outputs that inform actionable system outputs, interpretations flagged for refinement, and rejected interpretations that prevent erroneous outputs. The active learning framework 1933 enables continuous improvement through selective integration of validated operational data, high-confidence interpretations, and new behavioral correlates, implementing the progressive model refinement capabilities.

An advanced embodiment of the present invention incorporates transfer learning within complex machine learning frameworks to facilitate the accelerated deployment and enhancement of bilateral neural-based communication interfaces across diverse non-human animal species. Specifically, this embodiment leverages sophisticated neural network architectures pre-trained on extensive datasets derived from neural signal recordings of a representative animal species, such as canine subjects, spanning a broad spectrum of emotional states, cognitive engagements, and environmental contexts. These comprehensive datasets enable the initial neural network architecture to establish robust, generalized representations of neural signatures and their associated semantic interpretations.

When adapting the system for additional animal species, including but not limited to equines or felines, the invention implements targeted retraining strategies. Specifically, this involves selectively fine-tuning upper layers of the neural network architecture at higher learning rates, thus efficiently adapting the neural decoding mechanisms to accommodate species-specific neural pattern variations and cognitive contextual subtleties. Lower layers, preserving foundational features learned from extensive initial training, remain largely fixed, leveraging cross-species similarities in neural signal representation.

A salient aspect of this invention is its utilization of personalized adaptive calibration and continuous learning methodologies. Upon initial integration with an individual animal subject, a structured calibration protocol is employed wherein the animal experiences controlled, known stimuli (such as interactions involving play, provision of food, or exposure to mild stressors) while neural data is concurrently captured by the device. This rigorous calibration procedure establishes a personalized baseline neural dataset reflective of distinct neural correlates corresponding to specific emotional and cognitive states and associated behavioral responses particular to the individual animal.

Following calibration, the system applies an adaptive confidence-scoring algorithm during active deployment, continuously evaluating the certainty of neural signal interpretations in real time. Interpretations exceeding a predefined confidence threshold inform actionable system outputs, while lower-confidence interpretations are flagged for further refinement or ignored to prevent erroneous outputs. Additionally, the invention integrates an active learning framework through which neural decoding models progressively refine their interpretive precision by incorporating newly acquired operational data selectively validated through the confidence-scoring process. These refinements are carefully controlled, ensuring only high-confidence data is incorporated, thereby preserving model accuracy and minimizing the risk of degradation from unreliable or ambiguous inputs.

The modular architecture further facilitates scalability via specialized software modules, termed “language packs,” designed for efficient deployment across various species. These modules encapsulate tailored decoding schemes and species-specific sensory stimulus mappings, thus enabling rapid and flexible adaptation to new species without extensive retraining or hardware modifications.

Collectively, these sophisticated adaptive mechanisms and modular frameworks confer the invention with remarkable scalability, individualized precision, and long-term operational robustness, representing a significant advancement in the domain of interspecies neural communication technologies. The continuous, personalized learning capability uniquely positions this invention as a dynamic, context-aware, and self-evolving communication interface, precisely attuned to the evolving neural profiles of individual animals.

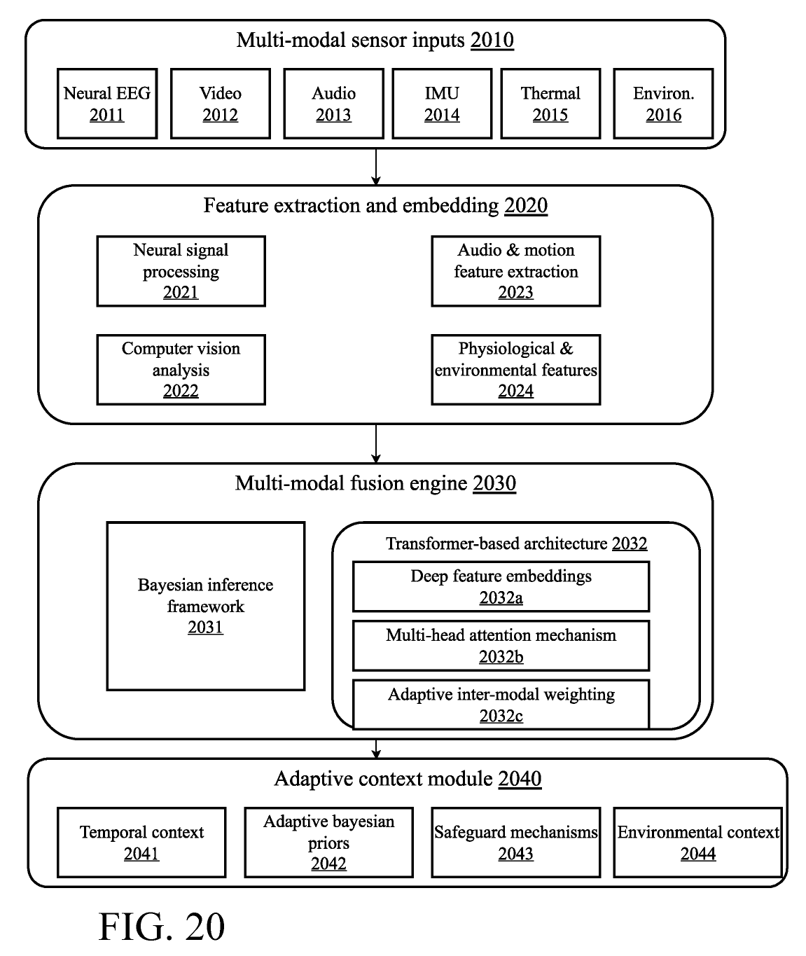

FIG. 20 is a block diagram illustrating a robust multi-modal fusion engine for interspecies communication system. The diagram systematically delineates a sophisticated four-tier hierarchical framework designed to achieve unprecedented accuracy in real-time interpretation of non-human animal states through context-aware integration of heterogeneous sensor data streams.

The multi-modal sensors inputs 2010 encompass six distinct and complementary sensing modalities. The neural EEG sensors 2011 capture fine-grained brain activity patterns and neurological signatures; the video imaging systems 2012 continuously monitor body posture, facial expressions, and behavioral indicators; the audio sensors 2013 detect and analyze vocalizations, utterances, and environmental acoustic contexts; the inertial measurement units (IMU)/accelerometers 2014 that precisely quantify movement intensity, gait patters, and physical activity levels; thermal imaging sensors 2015 that monitor physiological state changes through temperature variations; and environmental sensors 2016 providing comprehensive contextual information about the animal’s surroundings, including ambient conditions and spatial positioning. Each sensing modality is represented with a dedicated processing unit, indicating the system’s capacity for parallel multi-stream data acquisition.

The diverse data streams cascade downward into the second tier, feature extraction & embedding 2020 which implements specialized analytical processing pathways for each sensory modality. The advanced neural signal processing algorithms 2021 on raw EEG data, extracting frequency-domain features, event-related potentials, and neural synchronization patterns. The computer vision analysis 2022 algorithms process video input through techniques like pose estimation, motion tracking, and behavioral pattern recognition. The audio and motion feature extraction 2023 methods including spectral analysis, acoustic feature detection, and accelerometry signal processing. The physiological and environmental feature extraction 2024 through thermal gradient analysis and contextual parameter identification. Collectively, these processors transform heterogeneous raw sensor data into structured, normalize feature representations optimized for cross-modal integration, creating the foundation for the sophisticated fusion strategies.

The core multi-modal fusion engine 2030 presents two parallel implementation approaches. The Bayesian inference framework 2031 features a hierarchical probabilistic graphical model with three distinct layers: a top layer of four interconnected nodes representing the sensor evidence layer that processes direct inputs from feature extractors; a middle layer of three nodes forming the intermediate state layer that performs initial cross-modal integration; and a bottom-level node representing the final animal state estimation that produces a unified probabilistic assessment. This structure implements the “elaborate hierarchical probabilistic graphical model—a Bayesian network, where each distinct sensor modality provides evidence toward the network’s probabilistic hypotheses.

The alternative transformer-based architecture 2032 comprises three sequential processing stages: an upper layer for deep feature embeddings 2032a that creates rich representational vectors from each sensory modality; a middle layer implementing multi-head attention mechanisms 2032b that identifies correlations and salient patterns across diverse inputs through parallel attention computations; and a lower layer for adaptive inter-modal weighting 2032c that dynamically adjusts the relative importance of each sensory stream based on contextual relevance. This directly corresponds to the transformer-based neural architectures incorporating sophisticated attention mechanisms and contextually responsive attention mechanisms.

The adaptive context module 2040 continuously refines the system’s interpretive framework through four specialized components: temporal context processing 2041 that incorporates time of day, cyclical patterns, and historical behavioral records; adaptive Bayesian priors 2042 that algorithmically adjust probabilistic reasoning parameters based on learned individual animal patterns and behavioral consistencies; safeguard mechanisms 2043 implementing regularized updates, uncertainty-based sample weighting, and periodic recalibration procedures to prevent overfitting and contextual drift; and environmental context processing 2044 that integrates location awareness, ongoing activities, and social interaction data to establish comprehensive situational understanding. These collectively implement the integrated adaptive context module that employs temporal Bayesian updating and contextual adaptive priors to continuously refine interpretations.

In another exemplary embodiment of the invention, a robust and intelligent multi-modal fusion engine is presented, characterized by adaptive, context-sensitive interpretation of diverse sensor data streams to ensure highly accurate real-time communication between humans and non-human animals. This embodiment utilizes advanced Bayesian or transformer-based algorithms, dynamically integrating neural EEG signals, visual imagery, auditory inputs, inertial measurement unit (accelerometer) data, thermal imaging, and environmental sensors, thus offering a comprehensive assessment of the animal’s emotional, cognitive, and behavioral states.

At the core of this embodiment lies a sophisticated Bayesian inference framework that continuously updates its probabilistic reasoning based on evolving context. The system constructs an elaborate hierarchical probabilistic graphical model—a Bayesian network—where each distinct sensor modality provides evidence toward the network’s probabilistic hypotheses regarding the animal’s internal states. This hierarchical model allows for the probabilistic integration of heterogeneous data streams, dynamically assigning relevance weights to each modality based on real-time contextual cues.

For instance, upon the detection of heightened arousal in neural EEG readings, the Bayesian inference module evaluates complementary sensor inputs—such as accelerometer data for movement intensity, video analytics assessing body language, audio analysis identifying vocalizations, or thermal imaging indicating physiological changes—to accurately differentiate between genuine emotional excitement and physiological responses related to physical exertion. In cases where the neural EEG indicates excitement but accelerometer data simultaneously records vigorous physical activity, the model adaptively downweights the EEG arousal indicators to avoid misclassification, instead relying more heavily on complementary sensor streams.

Alternatively, the invention may leverage transformer-based neural architectures incorporating sophisticated attention mechanisms to achieve real-time adaptive fusion of sensor modalities. Each sensor data stream is first independently processed to extract deep feature embeddings, capturing distinct spatiotemporal characteristics inherent in neural, visual, auditory, and physiological sensor data. These feature embeddings are then integrated via contextually responsive attention mechanisms, which dynamically modulate inter-modal relevance weights in real-time based on temporal and contextual relationships detected within incoming data streams.

In such transformer-based embodiments, attention mechanisms are trained to identify correlations and predictive cues across diverse sensory inputs—such as video-based behavioral analysis, acoustic signals indicating vocalizations or environmental sounds, and physiological data including heart rate or thermal imaging signals. During operational scenarios, for instance, in periods of minimal physical activity detected by video and motion sensors, attention layers dynamically elevate reliance on EEG and subtle physiological signals, ensuring accurate interpretations of the animal’s mental and emotional states without interference from motion artifacts or environmental distractions.

Further enhancing this embodiment, an integrated adaptive context module employs advanced machine learning techniques, including temporal Bayesian updating and contextual adaptive priors, to continuously monitor and update contextual information—such as time of day, historical behavioral patterns, and environmental factors. For instance, if historical data indicate that the animal consistently experiences heightened excitement during specific routine events (e.g., feeding times or familiar social interactions), the system automatically adjusts its probabilistic priors and predictive weighting strategies to reflect these known patterns, thereby producing interpretations aligned with nuanced, individualized contexts rather than static sensor thresholds.

Moreover, the system includes safeguards within the adaptive updating mechanism to mitigate risks associated with overfitting or contextual drift. These safeguards include mechanisms such as regularized updates, uncertainty-based sample weighting, and periodic recalibration with baseline contextual parameters, ensuring that adaptations improve interpretive accuracy without introducing systematic biases or inaccuracies.

Through the integration of these advanced context-aware, multi-modal fusion strategies—including Bayesian hierarchical modeling, transformer-based attention mechanisms, and adaptive contextual learning—the invention significantly enhances interpretive accuracy, robustness, and reliability beyond that achievable by simpler threshold-based systems. Consequently, this embodiment establishes an innovative benchmark in the domain of interspecies communication technologies, offering unprecedented levels of interpretative fidelity, adaptability, and real-time responsiveness tailored specifically to the complex demands inherent in interspecies neural communication applications.

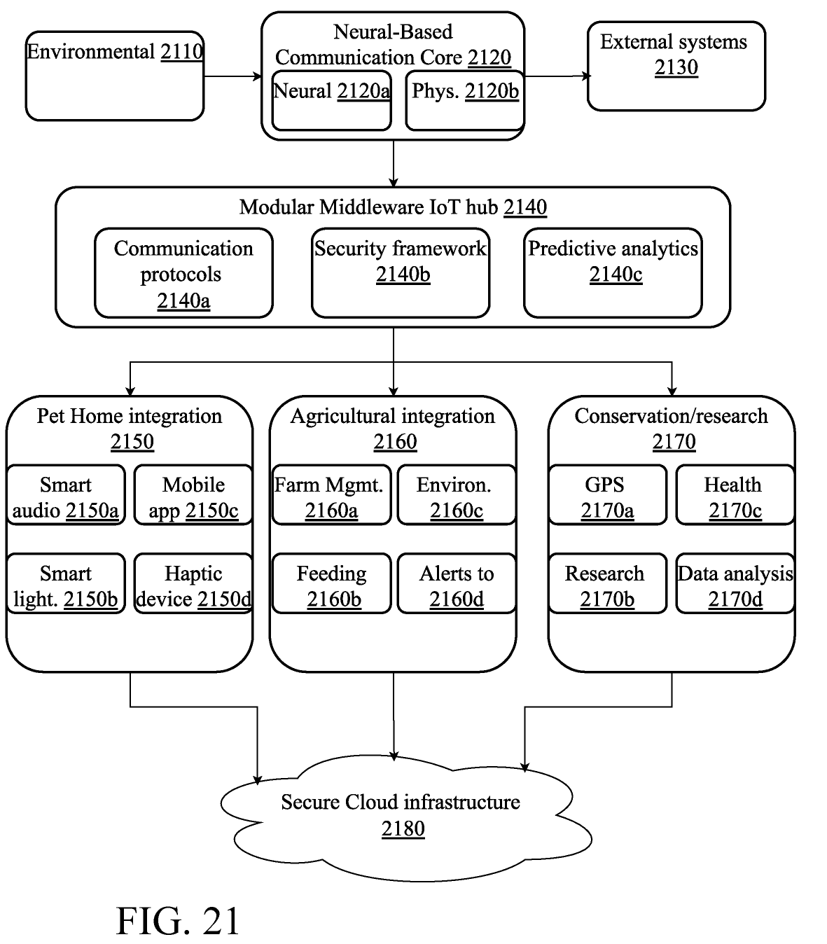

FIG. 21 is a block diagram illustrating an advanced IoT integration framework for neural-based animal communication. The subsystem transforms the neural-based animal communication apparatus from a discrete wearable technology into a sophisticated central integration node within a comprehensive internet of things ecosystem.

The neural-based communication core 2120 forms the foundation of the subsystem, encompassing primary input components: the neural interface module 2120a feature EEG sensors and advanced graphene e-tattoos for direct brain activity monitoring; and the physiological 2120b module capturing vital biometrics including heart rate and respiratory rate. The environmental module 2110 collecting contextual data such as temperature, ambient sound, and visual inputs. These components collectively gather the multimodal data streams that serve as the foundation for comprehensive animal state interpretation and provide to external systems 2130.

The modular middleware IoT hub 2140 forms the technological cornerstone. This middleware layer consists of three essential components: a robust communication protocols module 2140a supporting industry-standard protocols (MQTT, Zigbee, Z-Wave, Bluetooth LE, Wi-Fi, and RESTful APIs) to facilitate seamless interoperability with diverse third-party systems; an advanced security framework 2140b implementing encryption, secure authentication, and data privacy protection measures as specified; and a state-of-the-art predictive analytics module 2140c leveraging sophisticated machine learning architectures including recurrent neural networks (RNNs/LSTMs), convolutional neural networks (CNNs), transformer models, and neurosymbolic learning frameworks.

The pet home integration 2150 depicts the practical implementation, showing how the system interfaces with smart home devices including smart audio systems 2150a that deliver calming sounds, smart lighting 2150b for ambient environmental control, mobile applications 2150c for real-time owner alerts, and haptic devices 2150d providing tactile comfort through gentle feedback. The agricultural integration 2160 scenario visualizes the implementation in livestock settings, featuring connections to farm management software 2160a, automated feeding systems 2160b, environmental controls 2160c for optimizing living conditions, and alert systems for farm personnel 2160d. The conservation and research 2170 extends the system’s applicability to wildlife monitoring and research contexts, incorporating GPS tracking collars 2170a, research databases 2170b, health monitoring devices 2170c, and data analysis platforms 2170d.

All the integration scenarios connect to a secure cloud infrastructure 2180, representing the cloud-based data processing and storage capabilities. A prominent feedback loop extends from the cloud infrastructure back to the predictive analytics module, illustrating the system’s capability for ongoing refinement through adaptive machine learning mechanisms.

The comprehensive visualization effectively captures the sophisticated integration framework that positions the neural-based animal communication system as a “robust, intelligent integration nexus that markedly complements and enhances current animal welfare, management, and caregiving technologies.”

In an additional exemplary embodiment, the invention discloses an advanced integration framework serving as an extensive Internet of Things (IoT) hub, purposefully designed to interconnect seamlessly with pre-existing animal management systems and environmental control infrastructures. This embodiment transitions the neural-based animal communication apparatus from a discrete wearable technology into a central integration node within comprehensive IoT ecosystems, extensively utilized by pet caretakers, livestock administrators, conservation specialists, and veterinary experts.

Fundamental to this embodiment is a modular middleware platform architected explicitly to standardize data transmission protocols and interoperability among a myriad of third-party animal monitoring and management systems. By leveraging established IoT communication standards—including but not limited to MQTT, Zigbee, Z-Wave, Bluetooth Low Energy (BLE), and Wi-Fi—the neural communication interface facilitates seamless and secure bidirectional data exchanges with various external systems, including livestock management software, GPS-enabled animal tracking collars, and wearable health monitoring devices. Such integration markedly enriches the data streams available for advanced interpretation and provides actionable insights for improved animal care.

In practical pet management scenarios, the system operates by detecting heightened stress or anxiety states in an animal through sophisticated analysis of EEG neural patterns and associated physiological indicators. Upon recognition of these specific neural signatures, the system autonomously initiates context-sensitive responses via cloud-connected smart home services. For instance, it activates calming ambient auditory stimuli through integrated smart audio systems or modulates environmental lighting configurations utilizing interconnected smart lighting devices to foster a tranquil setting. Concurrently, the system conveys immediate alerts to pet owners through personalized smartphone applications, offering precise situational awareness and enabling swift, informed interventions remotely.

In agricultural settings, this IoT hub supports advanced operational decision-making by integrating real-time neural interface data with farm management software systems. If indicators of stress, health abnormalities, or behavioral anomalies are detected within livestock populations—ascertained through combined neural and physiological sensor analyses—the system automatically transmits relevant data into existing farm management dashboards. Subsequently, pre-programmed automated responses are triggered, including environmental adjustments, modification of nutritional schedules, or issuance of timely alerts to farm personnel, facilitating rapid evaluation and intervention to optimize animal health, welfare, and overall herd productivity.

The technological architecture integrates secure cloud-based middleware frameworks, utilizing robust IoT communication protocols such as MQTT and RESTful APIs, ensuring seamless and secure interoperability across diverse external devices and platforms. Rigorous cybersecurity provisions are incorporated, including advanced encryption methodologies and secure authentication protocols, to protect the privacy and integrity of animal-derived data throughout real-time data transmission and persistent storage.

Additionally, the embodiment incorporates sophisticated machine learning-driven contextual analytics, capable of intelligent predictive analytics and automated triggering of IoT ecosystem responses based on simultaneous evaluation of neural and environmental sensor inputs. Thus, the system not only amalgamates multiple data streams but also dynamically orchestrates intelligent, context-aware automated actions across interconnected smart devices and systems.

Through strategic positioning as an integrative IoT hub, the neural-based animal communication system substantially expands its operational utility, scalability, and applicability across numerous domains, providing a robust, intelligent integration nexus that markedly complements and enhances current animal welfare, management, and caregiving technologies.

The predictive module explicitly integrates historical and real-time multimodal data—including neural signals (e.g., EEG from flexible graphene e-tattoos), physiological signals (heart rate, respiratory rate, galvanic skin response), and environmental sensor inputs (temperature, humidity, ambient sound, visual analytics, atmospheric pressure, and air quality measurements)—to proactively anticipate animal emotional and behavioral states. This predictive engine utilizes advanced machine learning architectures such as recurrent neural networks (RNNs), convolutional neural networks (CNNs), transformer models, and hybrid neurosymbolic architectures trained on comprehensive datasets. These datasets correlate multimodal sensor inputs with annotated and empirically observed animal emotional states, providing a robust foundation for accurate forecasting of animal responses to varying environmental and internal stimuli.

In operation, the predictive module continuously processes incoming multimodal data streams, integrating them in real-time through sophisticated machine learning algorithms. Recurrent neural networks (RNNs) including long short-term memory (LSTM) or gated recurrent units (GRU) analyze these streams, identifying subtle temporal sequences and correlations predictive of future states such as anxiety, stress, excitement, or aggression. For instance, historical data associating neural signatures and environmental triggers—like thunderstorms, fireworks, or sudden loud noises—may be analyzed by the predictive algorithm to reliably anticipate anxiety episodes before overt behavioral manifestations become apparent. Additionally, convolutional neural networks (CNNs) and transformers further enhance the system’s predictive capabilities by effectively capturing spatial-temporal patterns and nuanced context within the complex interplay of sensor inputs.

Upon detecting high-confidence predictive signals indicative of potential emotional or behavioral disturbances, the predictive module initiates tailored and preemptive interventions designed to mitigate or even prevent the anticipated state. Such interventions are carefully calibrated according to the species-specific and individual animal’s known preferences, historical responses, and sensitivity thresholds. Examples include gentle haptic feedback through wearable devices to provide soothing tactile stimulation, calming auditory cues emitted through localized speakers or bone-conduction transducers, and controlled sensory inputs—such as adjustable lighting, temperature modulation, or environmental masking sounds—to immediately influence the animal’s sensory perception, thus effectively preempting escalation of negative emotional states.

Furthermore, the predictive module incorporates advanced adaptive machine learning mechanisms that ensure continuous improvement of predictive accuracy and reliability. These mechanisms systematically compare predicted states against actual observed emotional or behavioral outcomes, refining the system through incremental learning algorithms. Techniques such as semi-supervised learning, reinforcement learning, and neurosymbolic reasoning frameworks facilitate real-time updating of predictive models based on the ongoing accumulation of data. Discrepancies between predicted and actual states dynamically trigger recalibration, enabling the predictive models to progressively adapt to individual animals’ evolving neural, physiological, and behavioral profiles, as well as changing environmental conditions throughout their lifetimes.

Scalability and versatility of the predictive module are notably enhanced through sophisticated use of transfer learning methodologies and universal semantic embedding architectures. These machine learning techniques allow efficient adaptation and cross-species predictive capability by identifying and exploiting common emotional or behavioral signatures shared across multiple species. Universal semantic embeddings enable rapid calibration for new animal species, minimizing the requirement for extensive retraining by leveraging existing cross-species neural and behavioral patterns. Consequently, the predictive module may swiftly accommodate new species or individual animals by recalibrating existing models with minimal supplementary training data, significantly reducing deployment time and cost.

Additionally, to enhance contextual intelligence, the predictive module may utilize neurosymbolic reasoning frameworks and semantic knowledge bases, which explicitly encode domain-specific knowledge (e.g., typical stress triggers, common behavior cues, species-specific responses). Such frameworks allow the predictive algorithms to reason logically about sensor data, environmental contexts, and behavioral history, providing deeper understanding and more nuanced predictive capabilities than purely data-driven methods alone. Incorporating semantic and symbolic reasoning enables real-time interpretation of ambiguous or conflicting multimodal data inputs, significantly reducing false-positive predictions and ensuring actionable predictive insights tailored precisely to the animal’s current situational context.

Overall, by robustly integrating advanced machine learning techniques, adaptive continuous learning, and sophisticated environmental-contextual predictive models, this predictive module significantly advances real-time animal welfare management, improves operational readiness in working animal applications, and enhances effective cross-species communication systems. This combination ensures the invention remains a pioneering and versatile solution suitable for diverse applications such as veterinary care, wildlife conservation, animal-assisted therapeutic interventions, and operational management of working animals across various challenging scenarios and environments.

The predictive module explicitly integrates comprehensive historical and real-time multimodal data—including neural signals such as EEG captured through advanced flexible graphene e-tattoos, physiological metrics like heart rate variability and respiratory patterns, and detailed environmental sensor data including temperature fluctuations, humidity changes, ambient sound profiles, and visual analytics—to proactively anticipate animal emotional and behavioral states with high precision. To achieve this, the predictive engine harnesses sophisticated machine learning frameworks, including but not limited to recurrent neural networks (RNNs), long short-term memory (LSTM) networks, convolutional neural networks (CNNs), transformer architectures, and hybrid neurosymbolic models. These algorithms are rigorously trained on extensive historical datasets that comprehensively document correlations between neural signals, physiological indicators, environmental conditions, and the resultant behavioral and emotional outcomes.

The predictive system continuously aggregates and synthesizes these multimodal data streams in real-time, detecting intricate temporal patterns indicative of impending emotional or behavioral changes. For example, recurrent neural network models specifically trained on extensive historical correlations between neural signals such as EEG patterns and environmental stimuli—like thunderstorms, fireworks, or unexpected loud noises—may predict anxiety episodes well before these states become externally visible through overt behavioral signs. Upon detection of predictive signals surpassing predetermined confidence thresholds, the system autonomously executes preemptive, contextually tailored interventions that include calming haptic stimuli, auditory reassurance cues (such as species-specific calming tones), or carefully regulated sensory inputs. These interventions are customized not only by species but also tailored to individual animal preferences, thereby optimizing their effectiveness and ensuring timely mitigation of stress or anxiety episodes before overt behavioral signs emerge.

Moreover, the predictive framework employs adaptive feedback loops that continuously refine its predictive models through sophisticated reinforcement learning and semi-supervised learning methods. Each predictive output generated by the system is systematically correlated with subsequent observed animal behaviors or physiological responses to assess the accuracy and reliability of predictions. Confirmed predictions reinforce the existing model parameters, while deviations or inaccuracies trigger automatic retraining or recalibration through semi-supervised and reinforcement learning methods. Over iterative cycles of prediction, observation, and refinement, the system’s accuracy progressively improves, enabling dynamic adaptation to evolving individual animal behaviors, changing environmental contexts, and emerging patterns across species.

Further enhancing animal safety and welfare, the invention incorporates a robust real-time anomaly detection module. This module actively monitors streaming multimodal data, employing advanced algorithms such as Isolation Forest, autoencoder-based methods, and specialized time-series analysis techniques, including Prophet or equivalent statistical models, to detect deviations from established individual-specific baseline patterns. Such anomalies might signal neurological events like seizures, states of confusion, disorientation, acute distress, or other significant health episodes. By immediately identifying these deviations from baseline states, the anomaly detection system provides rapid, actionable insights enabling prompt intervention.

Upon anomaly detection, the system autonomously initiates contextually appropriate protective responses. For instance, characteristic EEG spikes indicative of seizure activity trigger the activation of calming interventions, including gentle auditory stimuli or soothing haptic signals, alongside notifications sent directly to animal handlers or veterinary personnel. Simultaneously, detailed event logging captures anomalies, interventions, and outcomes, enriching historical datasets and further refining predictive accuracy. Adaptive learning components within the anomaly detection module integrate handler and veterinary confirmations of system-detected anomalies to continuously optimize predictive specificity and sensitivity. This iterative learning ensures that critical events are reliably identified with minimal false alarms, thereby significantly bolstering the system’s practical utility.

Ultimately, the predictive and anomaly detection modules are designed for scalability and rapid adaptation across various animal species. Leveraging transfer learning, minimal additional calibration data may quickly recalibrate and fine-tune existing models to accommodate new species-specific neural patterns and behaviors. This flexibility enables extensive deployment in diverse operational environments, from veterinary clinics and wildlife monitoring initiatives to search-and-rescue operations and conservation management programs, solidifying the invention’s position as a pioneering, adaptable, and broadly applicable technological advancement in animal welfare and human-animal communication.

The invention explicitly integrates an advanced energy-harvesting embodiment designed specifically to enhance practical usability, particularly in long-term, field-based applications such as wildlife tracking or remote monitoring of free-ranging animals. The wearable device includes miniaturized energy-harvesting modules specifically designed to leverage various environmental and animal-generated energy sources, ensuring continuous operation without frequent manual intervention or battery replacements.

The system integrates kinetic energy harvesting units utilizing piezoelectric transducers strategically positioned within the wearable to capture mechanical energy generated from routine animal movements—including walking, running, or even subtle muscular or respiratory movements. These transducers convert mechanical strain from normal animal motion into electrical energy stored in compact, integrated storage solutions such as thin-film lithium-ion batteries or advanced supercapacitors. These components are optimized in size and shape to seamlessly conform to animal anatomy, minimizing interference with natural behaviors while effectively harnessing energy from motion.

In addition to kinetic harvesting, the embodiment incorporates flexible photovoltaic modules constructed from lightweight, durable, and weather-resistant organic photovoltaic (OPV) or thin-film solar materials positioned on exposed portions of the wearable device. These solar modules capture ambient solar radiation, supplementing kinetic harvesting, especially in outdoor field deployments with abundant natural sunlight exposure. The system intelligently manages this harvested solar energy through an embedded power management circuit, which dynamically allocates power to essential predictive, communication, and monitoring subsystems, optimizing operational longevity and reliability.

Thermoelectric generators (TEGs) integrated within the wearable exploit temperature differentials between the animal’s body heat and the ambient environment. These ultra-thin, flexible thermoelectric modules efficiently convert thermal gradients into electrical energy, providing continuous, supplemental power even in stationary or low-activity periods where kinetic energy harvesting may be minimal. A specialized energy management subsystem utilizes advanced low-power energy storage solutions such as thin-film batteries or supercapacitors to efficiently store and buffer harvested energy, maintaining consistent, reliable device operation across fluctuating environmental conditions and varying animal activity levels.

Furthermore, the system incorporates an intelligent power management architecture that dynamically balances energy harvesting, storage, and consumption based on real-time analysis of energy availability and device operational needs. This embedded intelligent power management unit (PMU) actively prioritizes essential functions, such as neural monitoring, predictive analytics, and anomaly detection modules, ensuring uninterrupted core functionality even during extended periods of limited energy input. Additionally, the PMU employs adaptive algorithms to optimize energy harvesting efficiency over time, leveraging reinforcement learning methods to predict energy harvesting opportunities based on historical environmental and behavioral patterns.

This fully integrated, self-sufficient energy harvesting architecture significantly distinguishes the invention from prior art dependent solely on conventional battery solutions. By autonomously managing energy sourced from kinetic, thermal, and solar inputs, the system achieves unparalleled operational longevity and reliability, particularly in remote or challenging field environments, enabling continuous, real-time neural and physiological monitoring with minimal human oversight or maintenance requirements.

### Exemplary Computing Environment

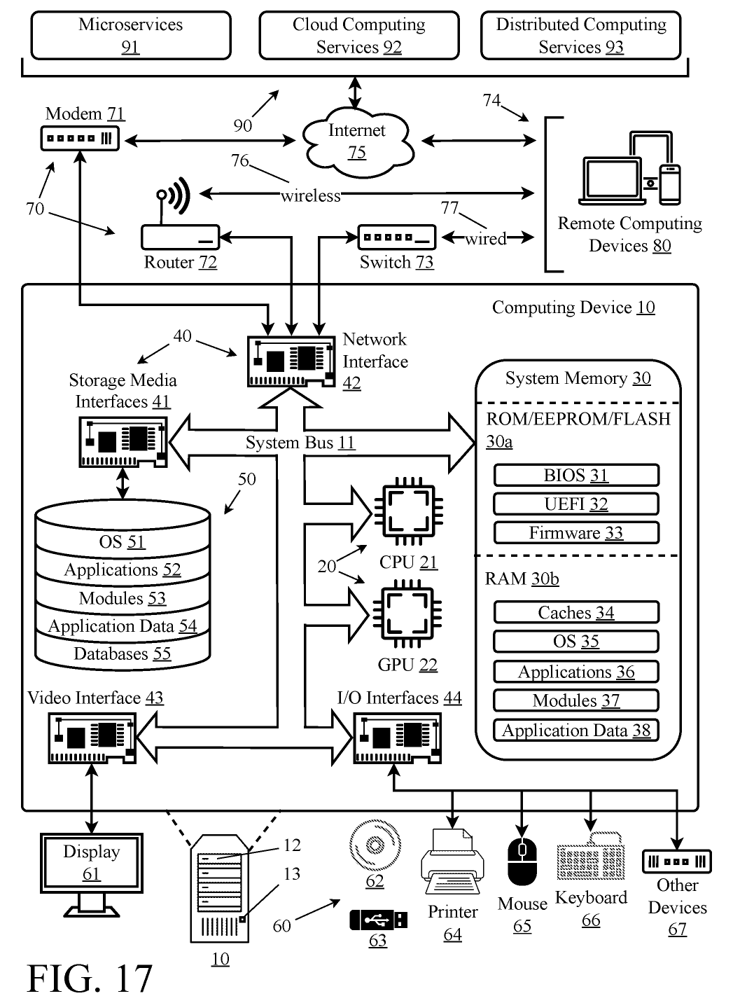

FIG. 17 illustrates an exemplary computing environment on which an embodiment described herein may be implemented, in full or in part. This exemplary computing environment describes computer-related components and processes supporting enabling disclosure of computer-implemented embodiments. Inclusion in this exemplary computing environment of well-known processes and computer components, if any, is not a suggestion or admission that any embodiment is no more than an aggregation of such processes or components. Rather, implementation of an embodiment using processes and components described in this exemplary computing environment will involve programming or configuration of such processes and components resulting in a machine specially programmed or configured for such implementation. The exemplary computing environment described herein is only one example of such an environment and other configurations of the components and processes are possible, including other relationships between and among components, and/or absence of some processes or components described. Further, the exemplary computing environment described herein is not intended to suggest any limitation as to the scope of use or functionality of any embodiment implemented, in whole or in part, on components or processes described herein.

The exemplary computing environment described herein comprises a computing device 10 (further comprising a system bus 11, one or more processors 20, a system memory 30, one or more interfaces 40, one or more non-volatile data storage devices 50), external peripherals and accessories 60, external communication devices 70, remote computing devices 80, and cloud-based services 90. System memory 30 may include a non-transitory, computer-readable medium.

System bus 11 couples the various system components, coordinating operation of and data transmission between those various system components. System bus 11 represents one or more of any type or combination of types of wired or wireless bus structures including, but not limited to, memory busses or memory controllers, point-to-point connections, switching fabrics, peripheral busses, accelerated graphics ports, and local busses using any of a variety of bus architectures. By way of example, such architectures include, but are not limited to, Industry Standard Architecture (ISA) busses, Micro Channel Architecture (MCA) busses, Enhanced ISA (EISA) busses, Video Electronics Standards Association (VESA) local busses, a Peripheral Component Interconnects (PCI) busses also known as a Mezzanine busses, or any selection of, or combination of, such busses. Depending on the specific physical implementation, one or more of the processors 20, system memory 30 and other components of the computing device 10 may be physically co-located or integrated into a single physical component, such as on a single chip. In such a case, some or all of system bus 11 may be electrical pathways within a single chip structure.

Computing device may further comprise externally-accessible data input and storage devices 12 such as compact disc read-only memory (CD-ROM) drives, digital versatile discs (DVD), or other optical disc storage for reading and/or writing optical discs 62; magnetic cassettes, magnetic tape, magnetic disk storage, or other magnetic storage devices; or any other medium which may be used to store the desired content and which may be accessed by the computing device 10. Computing device may further comprise externally-accessible data ports or connections 12 such as serial ports, parallel ports, universal serial bus (USB) ports, and infrared ports and/or transmitter/receivers. Computing device may further comprise hardware for wireless communication with external devices such as IEEE 1394 (“Firewire”) interfaces, IEEE 802.11 wireless interfaces, BLUETOOTH® wireless interfaces, and so forth. Such ports and interfaces may be used to connect any number of external peripherals and accessories 60 such as visual displays, monitors, and touch-sensitive screens 61, USB solid state memory data storage drives (commonly known as “flash drives” or “thumb drives”) 63, printers 64, pointers and manipulators such as mice 65, keyboards 66, and other devices 67 such as joysticks and gaming pads, touchpads, additional displays and monitors, and external hard drives (whether solid state or disc-based), microphones, speakers, cameras, and optical scanners.

Processors 20 are logic circuitry capable of receiving programming instructions and processing (or executing) those instructions to perform computer operations such as retrieving data, storing data, and performing mathematical calculations. Processors 20 are not limited by the materials from which they are formed or the processing mechanisms employed therein, but are typically comprised of semiconductor materials into which many transistors are formed together into logic gates on a chip (i.e., an integrated circuit or IC). The term processor includes any device capable of receiving and processing instructions including, but not limited to, processors operating on the basis of quantum computing, optical computing, mechanical computing (e.g., using nanotechnology entities to transfer data), and so forth. Depending on configuration, computing device 10 may comprise more than one processor. For example, computing device 10 may comprise one or more central processing units (CPUs) 21, each of which itself has multiple processors or multiple processing cores, each capable of independently or semi-independently processing programming instructions. Further, computing device 10 may comprise one or more specialized processors such as a graphics processing unit (GPU) 22 configured to accelerate processing of computer graphics and images via a large array of specialized processing cores arranged in parallel. The term processor may further include: neural processing units (NPUs) or neural computing units optimized for machine learning and artificial intelligence workloads using specialized architectures and data paths; tensor processing units (TPUs) designed to efficiently perform matrix multiplication and convolution operations used heavily in neural networks and deep learning applications; application-specific integrated circuits (ASICs) implementing custom logic for domain-specific tasks; application-specific instruction set processors (ASIPs) with instruction sets tailored for particular applications; field-programmable gate arrays (FPGAs) providing reconfigurable logic fabric that may be customized for specific processing tasks; processors operating on emerging computing paradigms such as quantum computing, optical computing, mechanical computing (e.g., using nanotechnology entities to transfer data), and so forth. Depending on configuration, computing device 10 may comprise one or more of any of the above types of processors in order to efficiently handle a variety of general purpose and specialized computing tasks. The specific processor configuration may be selected based on performance, power, cost, or other design constraints relevant to the intended application of computing device 10.

System memory 30 is processor-accessible data storage in the form of volatile and/or nonvolatile memory. System memory 30 may be either or both of two types: non-volatile memory and volatile memory. Non-volatile memory 30a is not erased when power to the memory is removed, and includes memory types such as read only memory (ROM), electronically-erasable programmable memory (EEPROM), and rewritable solid-state memory (commonly known as “flash memory”). Non-volatile memory 30a is typically used for long-term storage of a basic input/output system (BIOS) 31, containing the basic instructions, typically loaded during computer startup, for transfer of information between components within computing device, or a unified extensible firmware interface (UEFI), which is a modern replacement for BIOS that supports larger hard drives, faster boot times, more security features, and provides native support for graphics and mouse cursors. Non-volatile memory 30a may also be used to store firmware comprising a complete operating system 35 and applications 36 for operating computer-controlled devices. The firmware approach is often used for purpose-specific computer-controlled devices such as appliances and Internet-of-Things (IoT) devices where processing power and data storage space is limited. Volatile memory 30b is erased when power to the memory is removed and is typically used for short-term storage of data for processing. Volatile memory 30b includes memory types such as random-access memory (RAM), and is normally the primary operating memory into which the operating system 35, applications 36, program modules 37, and application data 38 are loaded for execution by processors 20. Volatile memory 30b is generally faster than non-volatile memory 30a due to its electrical characteristics and is directly accessible to processors 20 for processing of instructions and data storage and retrieval. Volatile memory 30b may comprise one or more smaller cache memories which operate at a higher clock speed and are typically placed on the same IC as the processors to improve performance.

Interfaces 40 may include, but are not limited to, storage media interfaces 41, network interfaces 42, display interfaces 43, and input/output interfaces 44. Storage media interface 41 provides the necessary hardware interface for loading data from non-volatile data storage devices 50 into system memory 30 and storage data from system memory 30 to non-volatile data storage device 50. Network interface 42 provides the necessary hardware interface for computing device 10 to communicate with remote computing devices 80 and cloud-based services 90 via one or more external communication devices 70. Display interface 43 allows for connection of displays 61, monitors, touchscreens, and other visual input/output devices. Display interface 43 may include a graphics card for processing graphics-intensive calculations and for handling demanding display requirements. Typically, a graphics card includes a graphics processing unit (GPU) and video RAM (VRAM) to accelerate display of graphics. One or more input/output (I/O) interfaces 44 provide the necessary support for communications between computing device 10 and any external peripherals and accessories 60. For wireless communications, the necessary radio-frequency hardware and firmware may be connected to I/O interface 44 or may be integrated into I/O interface 44.

Non-volatile data storage devices 50 are typically used for long-term storage of data. Data on non-volatile data storage devices 50 is not erased when power to the non-volatile data storage devices 50 is removed. Non-volatile data storage devices 50 may be implemented using any technology for non-volatile storage of content including, but not limited to, CD-ROM drives, digital versatile discs (DVD), or other optical disc storage; magnetic cassettes, magnetic tape, magnetic disc storage, or other magnetic storage devices; solid state memory technologies such as EEPROM or flash memory; or other memory technology or any other medium which may be used to store data without requiring power to retain the data after it is written. Non-volatile data storage devices 50 may be non-removable from computing device 10 as in the case of internal hard drives, removable from computing device 10 as in the case of external USB hard drives, or a combination thereof, but computing device will typically comprise one or more internal, non-removable hard drives using either magnetic disc or solid-state memory technology. Non-volatile data storage devices 50 may store any type of data including, but not limited to, an operating system 51 for providing low-level and mid-level functionality of computing device 10, applications 52 for providing high-level functionality of computing device 10, program modules 53 such as containerized programs or applications, or other modular content or modular programming, application data 54, and databases 55 such as relational databases, non-relational databases, object oriented databases, BOSQL databases, and graph databases.

Applications (also known as computer software or software applications) are sets of programming instructions designed to perform specific tasks or provide specific functionality on a computer or other computing devices. Applications are typically written in high-level programming languages such as C++, Java, and Python, which are then either interpreted at runtime or compiled into low-level, binary, processor-executable instructions operable on processors 20. Applications may be containerized so that they may be run on any computer hardware running any known operating system. Containerization of computer software is a method of packaging and deploying applications along with their operating system dependencies into self-contained, isolated units known as containers. Containers provide a lightweight and consistent runtime environment that allows applications to run reliably across different computing environments, such as development, testing, and production systems.

The memories and non-volatile data storage devices described herein do not include communication media. Communication media are means of transmission of information such as modulated electromagnetic waves or modulated data signals configured to transmit, not store, information. By way of example, and not limitation, communication media includes wired communications such as sound signals transmitted to a speaker via a speaker wire, and wireless communications such as acoustic waves, radio frequency (RF) transmissions, infrared emissions, and other wireless media.

External communication devices 70 are devices that facilitate communications between computing device and either remote computing devices 80, or cloud-based services 90, or both. External communication devices 70 include, but are not limited to, data modems 71 which facilitate data transmission between computing device and the Internet 75 via a common carrier such as a telephone company or internet service provider (ISP), routers 72 which facilitate data transmission between computing device and other devices, and switches 73 which provide direct data communications between devices on a network. Here, modem 71 is shown connecting computing device 10 to both remote computing devices 80 and cloud-based services 90 via the Internet 75. While modem 71, router 72, and switch 73 are shown here as being connected to network interface 42, many different network configurations using external communication devices 70 are possible. Using external communication devices 70, networks may be configured as local area networks (LANs) for a single location, building, or campus, wide area networks (WANs) comprising data networks that extend over a larger geographical area, and virtual private networks (VPNs) which may be of any size but connect computers via encrypted communications over public networks such as the Internet 75. As just one exemplary network configuration, network interface 42 may be connected to switch 73 which is connected to router 72 which is connected to modem 71 which provides access for computing device 10 to the Internet 75. Further, any combination of wired 77 or wireless 76 communications between and among computing device 10, external communication devices 70, remote computing devices 80, and cloud-based services 90 may be used. Remote computing devices 80, for example, may communicate with computing device through a variety of communication channels 74 such as through switch 73 via a wired 77 connection, through router 72 via a wireless connection 76, or through modem 71 via the Internet 75. Furthermore, while not shown here, other hardware that is specifically designed for servers may be employed. For example, secure socket layer (SSL) acceleration cards may be used to offload SSL encryption computations, and transmission control protocol/internet protocol (TCP/IP) offload hardware and/or packet classifiers on network interfaces 42 may be installed and used at server devices.

In a networked environment, certain components of computing device 10 may be fully or partially implemented on remote computing devices 80 or cloud-based services 90. Data stored in non-volatile data storage device 50 may be received from, shared with, duplicated on, or offloaded to a non-volatile data storage device on one or more remote computing devices 80 or in a cloud computing service 92. Processing by processors 20 may be received from, shared with, duplicated on, or offloaded to processors of one or more remote computing devices 80 or in a distributed computing service 93. By way of example, data may reside on a cloud computing service 92, but may be usable or otherwise accessible for use by computing device 10. Also, certain processing subtasks may be sent to a microservice 91 for processing with the result being transmitted to computing device 10 for incorporation into a larger processing task. Also, while components and processes of the exemplary computing environment are illustrated herein as discrete units (e.g., OS 51 being stored on non-volatile data storage device 51 and loaded into system memory 35 for use) such processes and components may reside or be processed at various times in different components of computing device 10, remote computing devices 80, and/or cloud-based services 90.

In an implementation, the disclosed systems and methods may utilize, at least in part, containerization techniques to execute one or more processes and/or steps disclosed herein. Containerization is a lightweight and efficient virtualization technique that enables packaging and running applications and their dependencies in isolated environments called containers. One of the most popular containerization platforms is Docker, which is widely used in software development and deployment. Containerization, particularly with open-source technologies like Docker and container orchestration systems like Kubernetes, is a common approach for deploying and managing applications. Containers are created from images, which are lightweight, standalone, and executable packages that include application code, libraries, dependencies, and runtime. Images are often built from a Dockerfile or similar, which contains instructions for assembling the image. Dockerfiles are configuration files that specify how to build a Docker image. Systems like Kubernetes also support containers or CRI-O. They include commands for installing dependencies, copying files, setting environment variables, and defining runtime configurations. Docker images are stored in repositories, which may be public or private. Docker Hub is an exemplary public registry, and organizations often set up private registries for security and version control using tools such as Hub, JFrog Artifactory and Bintray, Github Packages or Container registries. Containers may communicate with each other and the external world through networking. Docker provides a bridge network by default, but may be used with custom networks. Containers within the same network may communicate using container names or IP addresses.

Remote computing devices 80 are any computing devices not part of computing device 10. Remote computing devices 80 include, but are not limited to, personal computers, server computers, thin clients, thick clients, personal digital assistants (PDAs), mobile telephones, watches, tablet computers, laptop computers, multiprocessor systems, microprocessor based systems, set-top boxes, programmable consumer electronics, video game machines, game consoles, portable or handheld gaming units, network terminals, desktop personal computers (PCs), minicomputers, main frame computers, network nodes, virtual reality or augmented reality devices and wearables, and distributed or multi-processing computing environments. While remote computing devices 80 are shown for clarity as being separate from cloud-based services 90, cloud-based services 90 are implemented on collections of networked remote computing devices 80.

Cloud-based services 90 are Internet-accessible services implemented on collections of networked remote computing devices 80. Cloud-based services are typically accessed via application programming interfaces (APIs) which are software interfaces which provide access to computing services within the cloud-based service via API calls, which are pre-defined protocols for requesting a computing service and receiving the results of that computing service. While cloud-based services may comprise any type of computer processing or storage, three common categories of cloud-based services 90 are microservices 91, cloud computing services 92, and distributed computing services 93.

Microservices 91 are collections of small, loosely coupled, and independently deployable computing services. Each microservice represents a specific computing functionality and runs as a separate process or container. Microservices promote the decomposition of complex applications into smaller, manageable services that may be developed, deployed, and scaled independently. These services communicate with each other through well-defined application programming interfaces (APIs), typically using lightweight protocols like HTTP, gRPC, or message queues such as Kafka. Microservices 91 may be combined to perform more complex processing tasks.

Cloud computing services 92 are delivery of computing resources and services over the Internet 75 from a remote location. Cloud computing services 92 provide additional computer hardware and storage on as-needed or subscription basis. Cloud computing services 92 may provide large amounts of scalable data storage, access to sophisticated software and powerful server-based processing, or entire computing infrastructures and platforms. For example, cloud computing services may provide virtualized computing resources such as virtual machines, storage, and networks, platforms for developing, running, and managing applications without the complexity of infrastructure management, and complete software applications over the Internet on a subscription basis.

Distributed computing services 93 provide large-scale processing using multiple interconnected computers or nodes to solve computational problems or perform tasks collectively. In distributed computing, the processing and storage capabilities of multiple machines are leveraged to work together as a unified system. Distributed computing services are designed to address problems that cannot be efficiently solved by a single computer or that require large-scale computational power. These services enable parallel processing, fault tolerance, and scalability by distributing tasks across multiple nodes.

Although described above as a physical device, computing device 10 may be a virtual computing device, in which case the functionality of the physical components herein described, such as processors 20, system memory 30, network interfaces 40, and other like components may be provided by computer-executable instructions. Such computer-executable instructions may execute on a single physical computing device, or may be distributed across multiple physical computing devices, including being distributed across multiple physical computing devices in a dynamic manner such that the specific, physical computing devices hosting such computer-executable instructions may dynamically change over time depending upon need and availability. In the situation where computing device 10 is a virtualized device, the underlying physical computing devices hosting such a virtualized computing device can, themselves, comprise physical components analogous to those described above, and operating in a like manner. Furthermore, virtual computing devices may be utilized in multiple layers with one virtual computing device executing within the construct of another virtual computing device. Thus, computing device 10 may be either a physical computing device or a virtualized computing device within which computer-executable instructions may be executed in a manner consistent with their execution by a physical computing device. Similarly, terms referring to physical components of the computing device, as utilized herein, mean either those physical components or virtualizations thereof performing the same or equivalent functions.

The skilled person will be aware of a range of possible modifications of the various aspects described above. Accordingly, the present invention is defined by the claims and their equivalents.

What is claimed is:

1. A computer system comprising a hardware memory, wherein the computer system is configured to execute software instructions stored on nontransitory machine-readable storage media that:
receive non-human animal brainwave data;
process the non-human animal brainwave data through a machine-learning system, wherein the machine-learning system is configured to:
isolate one or more neural patterns in the non-human animal brainwave data;
associate the one or more neural patterns with an emotional state of a non-human animal;
compute a confidence score corresponding to the emotional state; and
in response to the confidence score exceeding a predetermined threshold, render and present an audio/visual form of the emotional state on an output device of the computer system.

2. The computer system of claim 1, further comprising a non-invasive brainwave sensor, wherein the non-invasive brainwave sensor is configured and disposed to obtain brainwave data from a non-human animal, and wherein the non-invasive brainwave sensor is configured to provide the brainwave data to the computer system.

3. The computer system of claim 2, wherein the non-invasive brainwave sensor comprises a sensor comprised of conductive polymer ink.

4. The computer system of claim 3, wherein the conductive polymer ink comprises (Poly(3,4-ethylenedioxythiophene):poly(styrenesulfonate)) (PEDOT:PSS).

5. The computer system of claim 2, wherein the non-invasive brainwave sensor comprises an epidermal tattoo sensor.

6. The computer system of claim 5, wherein the epidermal tattoo sensor comprises carbon nanotubes (CNTs).

7. The computer system of claim 5, wherein the epidermal tattoo sensor comprises gold nanomaterials.

8. The computer system of claim 2, wherein the non-invasive brainwave sensor further comprises a wireless data transmission module.

9. The computer system of claim 8, wherein the wireless data transmission module includes a Bluetooth Low Energy (BLE) module.

10. The computer system of claim 1, wherein the machine-learning system includes a large language model (LLM).

11. The computer system of claim 10, wherein the LLM includes a multi-head attention (MHA) mechanism.

12. The computer system of claim 1, wherein the computing system is further configured to:
receive and process brainwave data from multiple non-human animals;
coordinate task assignments among multiple non-human animals based on their detected emotional states; and
transmit adaptive commands to redistribute tasks in response to changes in cognitive load, stress levels, or environmental conditions.

13. The computer system of claim 1, wherein the machine-learning system is further configured to:
receive auxiliary sensor data comprising at least one of physiological data, motion data, audio data, video data, or thermal imaging data from the non-human animal;
process the auxiliary sensor data in conjunction with the brainwave data using cross-modal fusion techniques; and
adjust confidence scores for emotional state determinations based on correlations between the brainwave data and the auxiliary sensor data.

14. The computer system of claim 1, wherein computing the confidence score further comprises:
dynamically adjusting a confidence threshold based on at least one of: environmental context, time of day, the non-human animal’s baseline patterns, recent activity history, or signal quality metrics;
applying the dynamically adjusted confidence threshold to filter emotional state determinations; and
progressively refining the confidence threshold through continuous learning from feedback data comprising successful and unsuccessful emotional state interpretations.

15. A method for processing non-human animal brainwave data, the method comprising:
receiving non-human animal brainwave data;
processing the non-human animal brainwave data through a machine-learning system, wherein the machine-learning system is configured to:
isolate one or more neural patterns in the non-human animal brainwave data;
associate the one or more neural patterns with an emotional state of a non-human animal;
compute a confidence score corresponding to the emotional state; and
in response to the confidence score exceeding a predetermined threshold, rendering and presenting an audio/visual form of the emotional state on an output device.

16. The method of claim 15, further comprising obtaining brainwave data from a non-human animal using a non-invasive brainwave sensor, wherein the non-invasive brainwave sensor is configured to provide the brainwave data to a computing device.

17. The method of claim 16, wherein the non-invasive brainwave sensor comprises a sensor comprised of conductive polymer ink.

18. The method of claim 17, wherein the conductive polymer ink comprises (Poly(3,4-ethylenedioxythiophene): poly(styrenesulfonate)) (PEDOT:PSS).

19. The method of claim 16, wherein the non-invasive brainwave sensor comprises an epidermal tattoo sensor.

20. The method of claim 19, wherein the epidermal tattoo sensor comprises carbon nanotubes (CNTs).

21. The method of claim 19, wherein the epidermal tattoo sensor comprises gold nanomaterials.

22. The method of claim 16, further comprising wirelessly transmitting the brainwave data from the non-invasive brainwave sensor to the computing device.

23. The method of claim 22, wherein wirelessly transmitting the brainwave data includes using a Bluetooth Low Energy (BLE) module.

24. The method of claim 15, wherein processing the non-human animal brainwave data includes processing the data through a large language model (LLM).

25. The method of claim 24, wherein the large language model (LLM) includes a multi-head attention (MHA) mechanism.

26. The method of claim 15, further comprising:
receiving and processing brainwave data from multiple non-human animals;
coordinating task assignments among multiple non-human animals based on their detected emotional states; and
transmitting adaptive commands to redistribute tasks in response to changes in cognitive load, stress levels, or environmental conditions.

27. The method of claim 15, further comprising:
receiving auxiliary sensor data comprising at least one of physiological data, motion data, audio data, video data, or thermal imaging data from the non-human animal;
processing the auxiliary sensor data in conjunction with the brainwave data using cross-modal fusion techniques; and
adjusting confidence scores for emotional state determinations based on correlations between the brainwave data and the auxiliary sensor data.

28. The method of claim 15, wherein computing the confidence score further comprises:
dynamically adjusting a confidence threshold based on at least one of: environmental context, time of day, the non-human animal’s baseline patterns, recent activity history, or signal quality metrics;
applying the dynamically adjusted confidence threshold to filter emotional state determinations; and
progressively refining the confidence threshold through continuous learning from feedback data comprising successful and unsuccessful emotional state interpretations.
